(19) United States
(12) Patent Application Publication
(10) Pub. No.: US 2023/0144001 A1
(43) Pub. Date: May 11, 2023

(54) DECELLULARIZED VASCULAR GRAFTS, METHODS, AND BENCH-TOP MODELS OF ATHEROSCLEROSIS

(71) Applicant: Wayne State University, Detroit, MI (US)

(72) Inventor: Mai T. Lam, Canton, MI (US)

(73) Assignee: Wayne State University, Detroit, MI (US)

(21) Appl. No.: 18/045,126

(22) Filed: Oct. 7, 2022

Related U.S. Application Data
(60) Provisional application No. 63/254,090, filed on Oct. 9, 2021.

(51) Int. Cl. G01N 33/50; C12N 5/071; C12M 1/12; C12M 3/00

(52) U.S. Cl. CPC G01N 33/5082; C12N 5/0691; C12M 25/04; C12M 21/08; C12N 2533/40; C12N 2533/56; C12N 2513/00

(57) ABSTRACT

Described herein are decellularized extracellular matrix for mechanically supporting engineered vascular grafts. Methods are provided for fabricating all-natural, non-immunogenic, strong products that do not rely on common plastic supports. Also provided are bench-top models of atherosclerosis. Embodiments provide completely inclusive models that contain all steps of atherosclerosis, including late-stage disease processes. Example models utilize tissue engineered blood vessels (TEBV); stages of atherosclerosis are induced for instance by application of oxidized low-density lipoprotein (oxLDLs) (early-stage), followed by macrophage introduction (early-stage), and induction of calcification using calcified protein particles (CPPs; late-stage). Also provided are kits useful to investigate disease processes and better patient treatment options, including new drug development.

### CROSS-REFERENCE TO RELATED APPLICATION

This application claims priority to and the benefit of the earlier filing of U.S. Provisional Application No. 63/254,090, filed on Oct. 9, 2021, which is incorporated by reference herein in its entirety.

### FIELD OF THE DISCLOSURE

The present disclosure relates to vascular grafts including vascular grafts with improved structural support and strength, methods of making and use of such grafts; and bench-top models for instance of atherosclerosis.

### INTRODUCTION

Cardiovascular disease (CVD) affects about 92.1 million Americans currently, amounting to an estimated $316 billion in health and lost productivity costs. Approximately 800,000 Americans die of CVD each year. See, Benjamin et al., Circulation. 2017 Mar. 7; 135(10): e146-e603. An effective treatment is to replace failed blood vessels with autografts from the same patient. However, there is an inherent lack of vessels for self-donation due to available supply from disease.

Engineered vessels are a potential way to solve the need for supply of viable vascular grafts. But one major issue hindering engineered graft performance is lack of strength in the all-biological tubes used as the basis of the grafts. See, L'Heureux et al., Nat Med. 12(3):361-5, 2006; Syedain et al., Biomaterials. 32(3):714-22, 2011; Syedain et al., Tissue Eng. Part A. 20, 1726-1734, 2014; Lawson et al., Lancet. 387(10032):2026-34, 2016; Krawiec et al., J Vasc Surg. 66(3):883-890.e1, 2017; and Krawiec et al., Tissue Eng. Part A. 22(9-10):765-75, 2016. Primarily, two methods are used to increase strength. One method is to subject the grafts to perfusion strength conditioning for several weeks, which significantly elongates the time for graft manufacture. Another method is to integrate a stiff polymeric tube into the graft. However, this approach increases the chance of graft rejection due to a foreign body response.

Native vessels provide structural support via the tunica adventitia, which is primarily composed of a collagen network created by resident fibroblast cells. In order to increase vascular graft strength, therefore, it would be desirable to produce grafts with a collagen network similar to the native adventitia.

Typically, extracellular matrix (ECM) protein production by cells is promoted in vitro via stimulation with various growth factors. Commonly used collagen production stimulating growth factors include ascorbic acid and TGF-β. However, growth factor stimulation was not sufficient to promote adequate collagen production by fibroblasts. Accordingly, there is a need in the art for novel approaches to introduce ECM proteins into engineered tissues in order to generate functionality similar to native vessels.

### OVERVIEW OF THE DISCLOSURE

The present disclosure provides, in some embodiments, a method of producing an engineered vascular graft wherein the vascular components are integrated into a decellularized tissue matrix. The collagen network in the tissue matrix provides the engineered vascular graft a support structure with high vessel strength.

Also provided are patient decellularized extracellular matrix for mechanically supporting engineered vascular grafts. Decellularized extracellular matrices (ECM) have been used in engineered tissues as a scaffold. However, decellularized ECM has not previously been used in engineered tissues as a mechanical support material. Here, in a specific example, it is shown that application of decellularized ECM, specifically demonstrated with ECM from the skin dermis, provides mechanical strength to engineered vascular grafts. All-natural tissue engineered vascular grafts lack the mechanical strength to handle human blood pressures. Dermis tissue from patients are used in this application to minimize immune rejection. This application allows for fabrication of an all-natural, non-immunogenic, strong product without relying on common plastic supports that introduce negative foreign body reaction. See, for instance, Exhibit B of U.S. Provisional Application No. 63/254,090 (incorporated herein by reference).

Another embodiment is a bench-top model of atherosclerosis. Described herein is the creation and development of a benchtop, tissue engineered model of atherosclerosis. Animal models have previously been the primary model system used to study atherosclerosis, however they are costly, require long periods of time to complete studies, and are difficult to provide information on the disease progression. Previous benchtop models lack critical aspects of the pathophysiology of the disease, and hence are profoundly inaccurate. A main component missing from previous benchtop models is the pathology of late stage atherosclerosis.

Provided herein is a new, completely inclusive model that contains all the steps of atherosclerosis, including late stage disease processes. Tissue engineered blood vessel (TEBV) are used as the foundation of this model. To the TEBV, the stages of atherosclerosis are induced by application of oxidized low density lipoprotein (oxLDLs) (early stage), followed by macrophage introduction (early stage), and induction of calcification using calcified protein particles (CPPs; late stage). The use of CPPs to induce calcification in an atherosclerosis model is a unique approach not previously employed in the field. Through the use of PCR markers, evidence is described of late stage structures of foam cells which constitute the plaques found in atherosclerosis.

Also provided is a rapid model system that is readily able to be distributed (e.g., in a kit form) to laboratories, such as researchers to investigate disease processes and better patient treatment options, including but not limited to new drug development. See, for instance, Exhibit A of U.S. Provisional Application No. 63/254,090 (incorporated herein by reference).

It may be understood that both the foregoing general description and the following detailed description are exemplary and explanatory only and are not restrictive of the invention, as claimed.

### BRIEF DESCRIPTION OF DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of this specification, illustrate exemplary embodiments of the present disclosure and together with the description, serve to explain the principles of the disclosure. Some of the drawings submitted herein, or in accompanying document(s), may be better understood in color.

FIG. 1 is a schematic illustration of a process for producing engineered vascular grafts according to various embodiments.

FIG. 2 is photograph of a culture system used to produce components of presently disclosed grafts.

FIG. 3A is a micrograph of a stained tissue section of a vascular ring without dermis, produced as described in Example 1. FIG. 3B is a stained tissue section of a vascular ring with dermis (D), produced as described in Example 1.

FIG. 4A is photograph of dermis ring loaded into the INSTRON tensile tester. FIG. 4B is a graph illustrating the average stress-strain results for vascular rings produced without acellular tissue matrix in two types of culture medium formulated with different growth factors. FIG. 4C is a graph illustrating the average stress-strain results for vascular rings produced with acellular tissue matrix in two types of culture medium formulated with different growth factors.

FIG. 5. Diagram of engineered vessel fabrication protocol. The Ring Stacking Method (RSM) is outlined for single-cell and two-cell engineered rings and vessels. Monolayers of vascular cells are plated onto silicon elastomer-coated dishes with a central post. The bottom coating allows for the cell monolayer to detach from the bottom of the plate once formed. Self-organized aggregation of monolayer progresses towards the central post, creating a ring of vascular tissue. Single cell vessels are composed of either fibroblasts for the adventitia or smooth muscle cells for the media layer. Bilayer vessels combine the adventitia and media layers to form the complete graft.

FIGS. 6A-6B. Relative mRNA expression profiles of the patient-derived cells. Characterization of patient cells relative to (FIG. 6A) ASCs using mesenchymal stem cell markers of CD90⁺, CD105⁺, and CD45⁻; and (FIG. 6B) relative to smooth muscle markers smooth muscle alpha-2 actin (ACTA2), smoothelin, (SMTN), transgelin (TAGLN), and calponin-1 (CNN1). Mesenchymal markers positive markers of CD90 and CD105 were present in ASCs although CD45 was also present. Mesenchymal marker expression was lowest in HASMCs, indicating their more differentiated state comparatively. ASCs exhibited expression of muscle markers, likely due to their mesenchymal origins. Smoothelin smooth muscle contractile protein was highly expressed in ASC-SMC II and HASMC cells. *denotes significance between groups (p<0.05).

FIGS. 7A-7B. Smooth muscle differentiation of ASCs results in significant expression of smooth muscle contractile proteins. (FIG. 7A) Alpha smooth muscle actin (αSMA) and myosin light chain kinase (MYLK) were detected via immunofluorescence in ASCs differentiated by angiotensin II (ASC-SMC I) or by angiotensin II followed by smooth muscle growth media (ASC-SMC II), and in human aortic smooth muscle cells (HASMCs) controls. Fluorescent images of cells stained for αSMA (green), MYLK (red) and DAPI (blue) were captured at 20× magnification (scale bar=100 μm). (FIG. 7B) Quantification of average fluorescence signal intensity per cell area (n=70) for each marker shows a significant increase in smooth muscle contractile protein expression following differentiation for the ASC-SMC II group. *denotes significance between groups (p<0.05).

FIGS. 8A-8B. Functional assay results showed ASC-derived cells and HASMCs had similar contraction capacity. (FIG. 8A) Representative time-lapse images of hydrogels contracted by the cells over time. (FIG. 8B) Measurement of hydrogel area contraction by each cell type showed minimal difference in cell contraction capacity between ASCs, ASC-SMCs, and HASMCs suggesting ASCs are capable of contraction at a similar level to smooth muscle cells. *denotes significance between cell types at the designated time point. Scale bar=10 mm.

FIGS. 9A-9F. Bilayer vascular grafts cultured for 1-month composed of (FIGS. 9A-9C) ASC-SMCs and fibroblasts or (FIGS. 9D-9F) aortic SMCS and fibroblasts. (FIGS. 9B, 9E) Hematoxylin and eosin depict two separate cellular layers (scale bar=200 μm). Greater collagen fiber maturity shown by fluorescence of (FIG. 9C) the ASC-SMC layer under polarized light relative to (FIG. 9F) aortic SMCs (scale bar=200 μm). Lumen denoted as L.

FIGS. 10A-10M. Ring and vessel tensile mechanics reveal increased elasticity in ASC-based engineered tissues. Gross images of (FIG. 10A) ASC, (FIG. 10B) ASC-SMC I, (FIG. 10C) PtFib, (FIG. 10D) ASC-SMC I+PtFib Bilayer, and (FIG. 10E) HASMC rings. Luminal and side views of vessels constructed of (FIGS. 10F, 10G) ASC-SMC I+PtFib bilayers and (FIGS. 10H, 10I) HASMC+PtFib bilayers. Tensile test setup for an (FIG. 10J) ASC-SMC I+PtFib bilayer ring and (FIG. 10K) ASC-SMC II+PtFib bilayer vessel. Stress-strain curves of (FIG. 10L) engineered ring mechanics showed highest ultimate tensile strength for ASC and HASMC rings, with the former exhibiting more elasticity. Stress-strain curves of (FIG. 10M) engineered vessel mechanics showed highest strength in the ASC-SMC II bilayer vessels. Scale bar=6 mm.

FIGS. 11A-11T. Patient cell-based engineered tissues exhibit organized cellularity and extracellular matrix. Histological analysis of tissue rings composed of (FIGS. 11A-11D) ASC, (FIGS. 11E-11H) ASC-SMC, (FIGS. 11I-11L) HASMC, (FIGS. 11M, 11P) PtFib, and (FIGS. 11O-11T) ASC-SMC+PtFib bilayer. Tissue organization as revealed in H&E, Picrosirius Red, and Masson's Trichrome showed increased organization in ASC-derived and PtFib-derived engineered tissues. HASMC-derived engineered tissues showed irregularity in cellular content and organization. Verhoeff-Van Gieson stain showed minimal to no evidence of elastin fibers in the engineered tissues. L denotes lumen. Scale bar=200 μm.

FIGS. 12A-12J. ASC-based engineered tissues show marked αSMA expression and mature collagen. (FIGS. 12A-12E) Immunofluorescence analysis of smooth muscle contractile protein αSMA (green) levels shows comparable expression in ASC-SMC tissues compared to control HASMC tissues. (FIGS. 12F-12F) Polarized light analysis of rings show primarily green immature collagen fibers across the engineered tissues with some evidence of more mature yellow to orange fibers in the ASC-SMC tissues. (FIGS. 12A, 12F) ASC, (FIGS. 12B, 12G) ASC-SMC, (FIGS. 12C, 12H) PtFib, (FIGS. 12D, 12I) ASC-SMC+PtFib Bilayer, and (FIGS. 12E, 12J) HASMC. Scale bar=200 μm.

FIGS. 13A-13L. ASC-based engineered vascular tissues maintain cellular organization and content over time. Engineered vessel histology following 1 month in culture showed the ASC-SMC bilayer group retained cellular content compared to bilayers constructed of HASMCs. This result indicates that ASCs ideally balance cell proliferation and organization in engineered vascular tissue application. L denotes lumen. Scale bar=200 μm.

FIGS. 14A-14I. ASC-based patient-specific engineered bilayer vessels show higher αSMA expression and mature collagen compared to HASMC vessels. αSMA (green) immunofluorescence stain showed higher intensity in the ASC-SMC bilayer compared to HASMC bilayers. Polarized light showed more mature yellow, orange, and red fibers in the ASC-based vessels, similar to the femoral artery, whereas the HASMC vessel was primarily composed of green immature collagen fibers. L denotes lumen. Scalebar=200 μm.

FIGS. 15A-15E. Diagram showing the BEBV setup in the perfusion system which was mimicked in the computer model (FIG. 15A). The BEBV was 12 mm, and the length of the silicone inlet and outlet tubes were 15 mm, for a total length of 42 mm. Control (FIG. 15B) and CPP (FIG. 15C) engineered rings are shown. Control (FIG. 15D) and CPP (FIG. 15E) BEBVs shown inside of a tall plate. (Scale bar=42 mm)

FIGS. 16A-16F. Uniaxial tensile results for the engineered vascular rings. FIG. 16A) Control (blue; n=5) and CPP (orange; n=5) ring force-displacement results show 59.8±8.64 mN and 76.6±10.1 mN respectively. FIG. 16B) Control (blue) and CPP (orange) ring stress-strain data showing a steeper linear region for the CPP compared to the control. Comparison of FIG. 16C) elastic modulus, FIG. 16D) ultimate tensile strength, FIG. 16E) failure strain, and FIG. 16F) stiffness for both groups showing 6.71±1.88 kPa, 20.0±1.86 kPa, 362±66.8%, and 4.55±0.74 N/m for the control group, and 13.3±1.81 kPa, 26.3±4.19 kPa, 214±18.0%, and 7.56±1.16 N/m for the CPP group respectively. (* p<0.05, ** p<0.01, *** p<0.001)

FIGS. 17A-17F. Uniaxial tensile results for the engineered BEBV vessels. FIG. 17A) Control (blue; n=5) and CPP (orange; n=5) BEBV force-displacement results show 322±32.0 mN and 406±48.0 mN respectively. FIG. 17B) Control (blue) and CPP (orange) BEBV stress-strain data showing similarly to the CPP rings the CPP BEBV group displays a steeper linear region compared to that of the control BEBV. Comparison of FIG. 17C) elastic modulus, FIG. 17D) ultimate tensile strength, FIG. 17E) failure strain, and FIG. 17F) stiffness for both groups showing 12.1±1.23 kPa, 23.6±2.15 kPa, 425±71.0%, and 23.07±4.33 N/m for the control group, and 16.2±1.56 kPa, 29.6±3.40 kPa, 460±51.9%, and 28.43±3.95 for the CPP group respectively. (* p<0.05, ** p<0.01)

FIGS. 18A-18F. Uniaxial tensile test results for the human Common Iliac Artery (CIA) for a normal (n=1) and a diseased atherosclerotic (n=1) vessel. Force-displacement curves showing 15.3 N and of 0.949 N respectively (FIG. 18A). Stress-strain curves showing low ultimate tensile stress but steeper linear region in the atherosclerotic group compared to the control (FIG. 18B). Elastic modulus (FIG. 18C), ultimate tensile strength (FIG. 18D), failure strain (FIG. 18E), and stiffness (FIG. 18F) for both groups showing 148 kPa, 598 kPa, 514%, and 512.53 N/m for the normal CIA, and 230 kPa, 70.3 kPa, 52.2%, and 856.36 N/m.

FIGS. 19A-19F. Cross-section at 21 mm (middle of the 42 mm long bioreactor setup) at times of 0.25 s, and 0.5 s representing the peak and lower velocities respectively. Control (FIG. 19A) and CPP (FIG. 19B) BEBV flow velocity profiles under normal conditions. On the other hand, Control (FIG. 19C) and CPP (FIG. 19D) BEBV flow velocity profiles under 75% concentric occlusion conditions (dashed outline) where the average velocities appear to have increased due to the plague build-up. Control (FIG. 19E) and, CPP (FIG. 19F) BEBV flow velocities under 75% eccentric occlusion conditions (dashed outline) average velocities have increased compared to the normal conditions flow profiles. (Scalebar=3 mm)

FIGS. 20A-20E. A graph comparing the average velocities of the normal control (solid blue) and normal CPP (dashed orange) BEBVs at timesteps 0.25, 0.5, 0.75 and 1 s (FIG. 20A). Average velocities by time graph comparing concentric occlusion control (solid blue) and CPP (dashed orange) BEBVs (FIG. 20B). There is a significant increase in the average velocity at the peak timesteps for the CPP group. A graph comparing the average velocities of the eccentric occlusion control (solid blue) and CPP (solid blue) groups (FIG. 20C). This graph shows a slightly higher but not significant change in the velocity in the CPP group compared to the control group. A bar graph comparing the absolute mean difference of the average at maximum time points for the normal, eccentric occlusion and concentric occlusion to further show the difference between the control and CPP group for each condition (FIG. 20D). Comparison of the Reynolds number at different times for the normal control, normal CPP, eccentric occlusion control, eccentric occlusion CPP, concentric occlusion control and concentric occlusion CPP (FIG. 20E). (* p<0.05)

FIGS. 21A-21F. Surface pressure along the BEBV and silicone tubes versus time, at 0.25 s, and 0.5 s. Simulation images of the pressure of the control (FIG. 21A) and CPP (FIG. 21B) BEBV under normal conditions. Simulation images of the pressure of the control (FIG. 21C) and CPP (FIG. 21D) BEBV when a 75% concentric occlusion is present displaying a significant increase in pressure before and at the plaque during timestep 0.25 s. Pressure simulation images of the control (FIG. 21E) and CPP (FIG. 21F) BEBV when a 75% eccentric occlusion is present, also showing a significant increase in pressure before and at the occluded region. (Scalebar=20 mm)

FIGS. 22A-22G. Is a graph comparing the average total pressure of the normal control (solid blue) and CPP (dashed orange) BEBVs with respect to time (FIG. 22A). The average total pressures of the concentric occlusion condition comparing the control (sold blue) and CPP (dashed orange) groups with respect to time (FIG. 22B). A significant increase in pressure was observed in the peak timesteps for the CPP group compared to the control. The average total pressure of the eccentric occlusion condition comparing the control (solid blue) and CPP (dashed orange) BEBVs groups with respect to time (FIG. 22C). A significant increase in pressure was observed in the peak timesteps for the CPP group compared to the control. A bar graph comparing the absolute mean pressure difference of the average at maximum time points for the normal and occluded conditions to further observe the difference between the control and CPP groups (FIG. 22D). Graph comparing the wall shear stress of the control and CPP BEBV groups at different times under normal (FIG. 22E), eccentric occlusion conditions (FIG. 22F) and concentric occlusion conditions (FIG. 22G). A significant increase in shear stress in present in all conditions at all timesteps. (** p<0.01, ***p<0.001)

FIGS. 23A-23I. Total Deformation of a longitudinal section of the control (FIG. 23A) and CPP (FIG. 23B) of the BEBV under normal conditions, control (FIG. 23C) and CPP (FIG. 23D) of the BEBV under concentric occlusion conditions, and control (FIG. 23E) and CPP (FIG. 23F) of the BEBV under eccentric occlusion conditions. Comparison of the average deformation of control (blue) and CPPs (orange) groups over each time step under normal, concentric and eccentric conditions (FIG. 23G). Results show 0.653±0.0405 mm (normal control), 0.530±0.0366 mm (normal CPP), 0.519±0.139 mm (concentric control), 0.484±0.153 mm (concentric CPP), 0.498±0.0750 mm (eccentric control) and 0.462±0.0790 mm (eccentric CPP). Comparison of the average strain over each time step of control (blue) and CPP (orange) groups over each time step under normal, concentric and eccentric conditions (FIG. 23H). Results show 0.254±0.0140 mm/mm (normal control), 0.217±0.0100 mm/mm (normal CPP), 0.326±0.0760 mm/mm (concentric control), 0.306±0.0790 mm/mm (concentric CPP), 0.334±0.0240 mm/mm (eccentric control), and 0.308±0.0280 mm/mm (eccentric CPP). Comparison of the average stress over each time step of control (blue) and CPP (orange) groups over each time step under normal, concentric and eccentric conditions (FIG. 23I). Results show 1760±129 Pa (normal control), 1740±126 Pa (normal CPP), 1787±345 Pa (concentric control), 1840±388 Pa (concentric CPP), 1660±208 Pa (eccentric control) and 1690±234 Pa (eccentric CPP). (Scalebar=20 mm, ** p<0.01, ***p<0.001) (Pinnock et al., 2020)

FIGS. 24A-24F. Tensile force comparison graph (FIG. 24A) comparing the engineered disease media (EDM) ring (grey) to the control (blue) and CPP (orange) ring tensile data present in FIGS. 16A-16F. Stress-strain comparison graphs comparing the EDM to control and CPP rings (FIG. 24B). Bar graphs are presented comparing the elastic modulus (FIG. 24C), ultimate tensile strength (FIG. 24D), failure strain (FIG. 24E), and stiffness (FIG. 24F) of the EDM rings to the control and CPP rings. (* p<0.05, ** p<0.01, *** p<0.001)

FIGS. 25A-25F. An image of an EDM ring in culture (FIG. 25A). H&E stain of the EDM ring at 4× (FIG. 25B). Von Kossa stain was performed to visualize the CPPs embedded inside of the hydrogel at 4× (FIG. 25C). A close up 10× image shows CPPs (black arrows) in abundance in the hydrogel (FIG. 25D). ox-LDLs embedded in the hydrogel were visualized via Oil Red O stain at 4× (FIG. 25E). A 20× image focused on a monocyte (black arrow) that has engulfed some ox-LDL that in the hydrogel (FIG. 25F). (4× Scalebar=1000 μm, 10× Scalebar=400 μm, 20× Scalebar=200 μm)

FIGS. 26A-26H Natural disease (ND) vessel (FIG. 26A) and ND vessel after static rotational macrophage seed (FIG. 26B). Oil Red O stain at 4× is presented (FIG. 26C) alongside a 40× close up of the same region to observe a macrophage that has migrated into the media layer and engulfed an ox-LDL particle (black arrow) (FIG. 26D). A hole in the sample created by macrophage migrating and engulfing the ox-LDL in the fibrin gel (FIG. 26E). An observation of the red ox-LDL particle with cells that appear to be macrophages in the same region (black arrow) (FIG. 26F). The same region at 40× show the macrophages engulfing the ox-LDL that was most likely in place of the hole prior to macrophage migration (FIGS. 26G and 26H). (4× Scalebar=1000 μm, 10× Scalebar=400 μm, 40× Scalebar=100 μm)

FIGS. 27A-27D Von Kossa stain of natural disease (ND) at 20× after static rotational macrophage seeding. Presence of CPPs present in the lumen of the ND vessel (black arrow) (FIG. 27A). Calcium deposits can be seen in the hydrogel and in the extracellular matrix of the smooth muscle cells (black arrows) (FIGS. 27B-27D). (20× Scalebar=200 μm)

### DETAILED DESCRIPTION

Reference will now be made in detail to certain exemplary embodiments according to the present disclosure, certain example of which are illustrated in the accompanying drawing.

Described herein are decellularized extracellular matrix for mechanically supporting engineered vascular grafts. Methods are provided for fabricating all-natural, non-immunogenic, strong products that do not rely on plastic supports.

Also provided are bench-top models of atherosclerosis. Embodiments provide completely inclusive models that contain all steps of atherosclerosis, including late stage disease processes. Example models utilize tissue engineered blood vessels (TEBV); stages of atherosclerosis are induced for instance by application of oxidized low density lipoprotein (oxLDLs) (early stage), followed by macrophage introduction (early stage), and induction of calcification using calcified protein particles (CPPs; late stage). Also provided are kits useful to investigate disease processes and better patient treatment options, including new drug development.

Provided herein is a method of producing a vascular graft with improved structural support and strength. Specifically, a group of cells (e.g. human dermal fibroblasts) are co-cultured with an acellular tissue matrix that provides improved structural support for the graft. The cells are also co-cultured with a hydrogel that comprises a structural protein (e.g. fibrin) and at least one component (e.g. TGF-β and ascorbic acid) that facilitates the synthesis of extracellular matrix proteins by the cells.

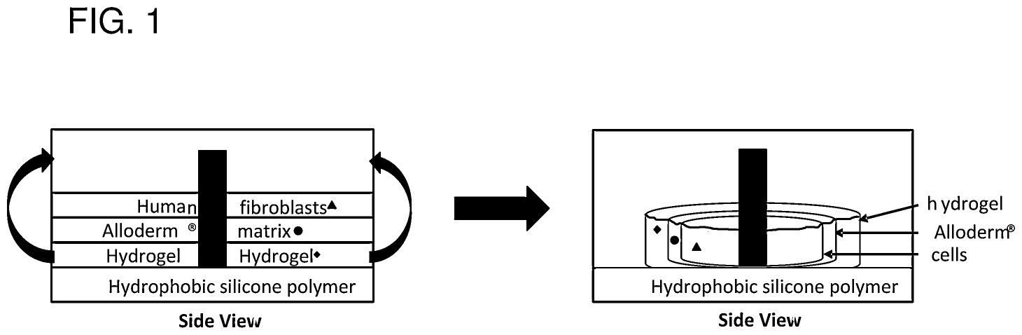

As illustrated in FIG. 1, the group of cells, the acellular tissue matrix, and the hydrogel are co-cultured in the same container (e.g., a tissue culture plate). The container may further comprise a supporting post that allows the assembly of the components into a ring-shaped structure. The acellular tissue matrix may be processed into a ring-shaped piece to be positioned around the supporting post. Either the acellular tissue matrix or the hydrogel is placed on the bottom of the container, and the other is positioned on top. The group of cells, which are in a suspension, are added to the container in contact with the layers of acellular tissue matrix and hydrogel. During co-culturing, the cells self-assemble into a ring-shaped cellular structure around the supporting post, and the cellular structure integrates into the layers of acellular tissue matrix and hydrogel to form a vascular graft with improved strength provided by the collagen network in the tissue matrix and the newly synthesized ECM proteins by the cells. The vascular graft produced by the method described herein is ring-shaped and has a lumen diameter similar to that of the supporting post. The innermost layer of the vascular graft is the cellular structure formed by the group of cells. The tissue matrix and hydrogel form the middle and outermost layers of the graft, with various possible arrangements.

The method described herein can be used to produce multiple ring-shaped vascular grafts, which can be further stacked into a tubular, vascular-like structure. The vascular graft produced by the methods described herein has improved vessel strength that is similar to a native vessel.

In one aspect, the current disclosure provides a method of producing a vascular graft comprising: selecting a group cells; selecting an acellular tissue matrix and a hydrogel; and co-culturing the group of cells with the acellular tissue matrix and the hydrogel in a container; thereby producing the vascular graft.

In certain embodiments, the cells used in the disclosed method are fibroblasts. The cells can be derived from human or animal blood vessels, or other tissues having similar cell types.

Native vessels have several layers, including the tunica adventitia, which includes fibroblasts. Its dense collagen I network functions as a structural support system to prevent rupture from increased pressure during blood flow. In certain embodiments, the vascular graft described herein is an adventitia graft. In certain embodiments, the vascular cells are human dermal fibroblasts. In certain embodiments, the human dermal fibroblasts are cultured in a growth medium and trypsinized.

Also described herein are embodiments that enable fabrication of the “tunica media” which is composed of “human smooth muscle cells.”

In certain embodiments, the current disclosure provides a method of producing a vascular graft that comprises self-assembled fibroblasts as the main cellular component. Optionally, grafts containing smooth muscle cells are also provided. Other suitable cell types can also be used in the disclosed method to produce vascular grafts. In one exemplary embodiment, the vascular cells used in the disclosed method are cultured cell lines, e.g., human dermal fibroblasts. Cells can be passaged multiple times prior to use. In certain embodiments, the cells are passaged 5-13 times prior to use. In certain embodiments, the cells are passaged no more than 20 times prior to use. In certain embodiments, the cells are adhesive cells and need to be trypsinized prior to passaging or co-culturing with the acellular dermal matrix and hydrogel. In certain embodiments, the cells are in a suspension prior to co-culture.

As used herein, the term “acellular tissue matrix” refers to an extracellular matrix derived from human or animal tissue, wherein the matrix retains a substantial amount of natural collagen and glycoproteins needed to serve as a scaffold to support attachment, growth, and assembly of the vascular cells. In certain embodiments, the acellular tissue matrix is a collagen-containing tissue matrix. “Acellular tissue matrices” are different from purified collagen materials, such as acid-extracted purified collagen, which are substantially void of other matrix proteins and do not retain the natural micro-structural features of tissue matrix due to the purification processes. Although referred to as “acellular tissue matrices,” it will be appreciated that such tissue matrices may combine with exogenous cells, including, for example, human dermal fibroblasts, or cells from a patient in whom the vascular graft may be implanted. For example, the self-assembled human fibroblasts will integrate into the acellular tissue matrix during co-culturing in order to form the vascular grafts with improved vessel strength. During or after implantation, the acellular tissue matrix in the vascular graft may be further in contact and integrated with cells and tissues in the patient.

“Acellular” or “decellularized” tissue matrices are understood to refer to tissue matrices in which no cells are visible using light microscopy.

The acellular tissue matrix from dermal or other tissues have been processed to remove at least some of the cellular components. In some cases, all, or substantially all, cellular materials are removed, thereby leaving corresponding extracellular matrix proteins. In certain embodiments, the acellular tissue matrix is completely decellularized. While dermal tissue is primarily described herein as being the source tissue for exemplary embodiments of acellular tissue matrix, it should be appreciated that the acellular tissue matrix described herein can originate from other tissue types. Other exemplary tissue types include, but are not limited to: adipose tissue, small intestine submucosa (SIS) tissue, muscle tissue, vascular tissue, and bone tissue.

The source tissues described herein may be derived from human or animal sources. For example, tissue may be obtained from cadavers. In addition, human tissue could be obtained from live donors (e.g., autologous tissue). Tissue may also be obtained from animals such as pigs, monkeys, or other sources. If animal sources are used, the tissues may be further treated to remove antigenic components such as galactose-alpha-1,3-galactose moieties, which are present in pigs and other mammals, but not humans or primates. In addition, the tissue may be obtained from animals that have been genetically modified to remove antigenic moieties. See Xu et al., Tissue Engineering, 15:1-13, 2009.

ALLODERM® and STRATTICE® (LifeCell Corporation, an AbbVie Company; Allergan Aesthetics) are two dermal acellular tissue matrices (also referred to as regenerative tissue matrices) made from human and porcine dermis, respectively. ALLODERM® and STRATTICE® dermal acellular tissue matrices have been used to assist in the treatment of structural defects and/or to provide support to tissues (e.g., for abdominal walls or in breast reconstitution), and their strength and biological properties make them well suited for such uses.

In certain embodiments, the acellular tissue matrix used in the disclosed method is ALLODERM®. The use of ALLODERM® as a vascular tissue support increases engineered vessel strength towards the physiological range, without the need for weeks of perfusion conditioning or polymer struts. Furthermore, ALLODERM® provides a 3D ECM milieu familiar to fibroblasts, to which resident fibroblasts preferentially attach to binding sites on the collagen network. Advantageously, the ADM retains intact fibrillary collagen, collagen IV, elastin, hyaluronan, proteoglycans, fibronectin, and vascular channels, which in combination efficacy support new tissue growth and maturation in the vascular graft.

In certain embodiments, the acellular tissue matrix is sterilized (e.g., by exposure to UV light) prior to co-culturing with the vascular cells and hydrogel. The acellular issue matrix may be further processed to different shapes and sizes to provide better structural and spatial support for the growth and assembly of the vascular cells. In certain embodiments, the acellular tissue matrix is cut into ring-shaped pieces prior to co-culturing with the cells. The ring-shaped pieces can have an inner diameter of about 5 mm, about 5.5 mm, about 6 mm, about 6.5 mm, about 7 mm, or about 7.5 mm and an outer diameter of about 9 mm, about 9.5 mm, about 10 mm, about 10.5 mm, about 11 mm, about 11.5 mm, about 12 mm, about 12.5 mm, or about 13 mm. The thickness of the ring-shaped acellular tissue matrix piece can be about 0.5 mm, about 1 mm, about 1.5 mm, about 2 mm, about 2.5 mm, or about 3 mm. It should be appreciated that the foregoing examples are not intended to be limiting. The acellular tissue matrix can be processed to any other suitable shapes and sizes according to different types of vascular grafts being produced.

To produce the vascular graft, the group of cells and the acellular tissue matrix are co-cultured with a hydrogel. In certain embodiments, the hydrogel comprises a structural protein and at least one components that facilitates the production of extracellular matrix proteins. In certain embodiments, the structural protein is fibrin. Fibrin hydrogels can be formed using thrombin, fibrinogen, and hydrogel medium comprising one or more components that facilitates the production of extracellular matrix protein, e.g., growth factors. Exemplary growth factors include, but are not limited to recombinant human insulin (rH-insulin), recombinant human fibroblast growth factor (rH-FGF), recombinant human epidermal growth factor (rH-EGF), transforming growth factor-beta (TGF-β), and ascorbic acid. In certain embodiments, the hydrogel comprises TGF-β and ascorbic acid. Appropriate concentrations of TGF-β and ascorbic acid can be, for example, about 0.05%, 0.06%, 0.07%, 0.08%, 0.09%, 0.1%, 0.15%, 0.2%, 0.25%, 0.3%, 0.35%, 0.4%, 0.45%, or 0.5%. Other suitable components that facilitate the growth, proliferation and production of functional proteins of the vascular cells can also be used to formulate the hydrogel.

To produce the vascular graft, the group of cells, the acellular tissue matrix and the hydrogel are co-cultured in a suitable medium, and in the same container. In certain embodiments, the culture medium contains one or more components that facilitate the production of extracellular matrix proteins, e.g., TGF-β and ascorbic acid. In certain embodiments, the container is a tissue culture plate or a tissue culture flask. To provide support for the assembly of the cells, the container may comprise an affixed supporting post. In one exemplary embodiment, the supporting post is a cylinder-shaped center post affixed to the bottom of the tissue culture plate, and the group of cells will undergo self-assembly around the center post, resulting in a ring-shaped vascular graft. The diameter of the supporting post decides the lumen size of the vascular graft. For example, the diameter of the center post can be about 5 mm, about 5.5 mm, about 6 mm, about 6.5 mm, about 7 mm, or about 7.5 mm. In certain embodiments, the container and the center post are sterilized prior to use.

The group of cells, the acellular tissue matrix, and the hydrogel can be positioned in various ways in order to achieve optimal integration of all components. In certain embodiments, the cells are trypsinized and added to the container in a suspension. In certain embodiments, the ring-shaped acellular tissue matrix is positioned on the bottom of the container, around the center post affixed to the bottom of a tissue culture plate and the hydrogel is added as a layer on top of the acellular tissue matrix. In certain embodiments, the hydrogel forms a layer on the bottom of the container and the ring-shaped acellular tissue matrix is positioned around the center post of the container, and on top of the hydrogel. In the container, the acellular tissue matrix and the hydrogel are in contact with each other and provide a scaffold for the cells to assemble and integrate into the matrix and the hydrogel, resulting in a vascular graft that has improved vessel strength.

In certain embodiments, the methods described herein can be used to produce multiple ring-shaped vascular grafts. The multiple ring-shaped vascular grafts can be further stacked or otherwise combined into a tubular, vascular-like structure.

Also described herein is a vascular graft that has improved structural support and vessel strength. In one aspect, disclosed herein is a vascular graft comprising a group of self-assembled cells, a collagen-containing tissue matrix; and a hydrogel comprising a structural protein and at least one component that facilitates the production of extracellular matrix proteins.

The vascular graft comprises a group of self-assembled cells, such as human fibroblasts integrated with an acellular tissue matrix and a hydrogel. In certain embodiments, the cells are fibroblasts (e.g., human dermal fibroblasts). In certain embodiments, the acellular tissue matrix in the vascular graft is derived from dermal tissue. In certain embodiments, the acellular tissue matrix is completely or substantially depleted of galactose alpha-1,3-galactose moieties. In certain embodiments, the hydrogel in the vascular graft comprises fibrin. Other structural proteins can also be used to form the hydrogel. In certain embodiments, the fibrin hydrogel comprises the hydrogel comprises ascorbic acid and transforming growth factor-beta (TGF-β).

In certain embodiments, the vascular graft is ring-shaped, with a diameter of about 6 mm. Multiple vascular grafts can be stacked to form a tubular vascular-like structure. In certain embodiments, the vascular graft has a layer of assembled fibroblasts facing the lumen of the graft, and a layer of hydrogel comprising a structural protein (e.g., fibrin) surrounding the assembled fibroblasts. The outermost layer of the graft is a layer of acellular tissue matrix. Various arrangements of the components in the vascular graft can be obtained by positioning the components in various ways during co-culturing. All components in the vascular graft are integrated with each other, providing superior structural support for the graft.

In certain embodiments, the vascular graft comprises at least one extracellular matrix protein synthesized by the vascular cells. Exemplary extracellular matrix proteins include, but are not limited to, fibrillary collagen, collagen IV, elastin, hyaluronan, proteoglycans, fibronectin. In certain embodiments, the extracellular matrix protein is fibrillary collagen.

The vascular graft described herein has mechanical properties that are similar to a native vessel. Various methods can be used to test the mechanical properties of the vascular graft. For example, tensile testing can be done to determine the elastic modulus, ultimate tensile strength, and failure strength of the graft. Other methods known to those skilled in the art can also be used to test the mechanical properties of the vascular graft.

The disclosed method circumvents the issue of stimulating cells alone (e.g., with growth factors) to produce enough protein in vitro, which has been proved difficult. In the present disclosure, the addition of a decellularized human extracellular matrix (ECM) into the engineered vascular graft provides high strength as compared to grafts produced with other methods.

The disclosed methods and products are particularly useful for treating cardiovascular diseases that require replacement of failed blood vessels. More generally, the herein-described vessels may be applied to vascular disease in general, such as peripheral artery disease (PAD). The disclosed vascular graft is biocompatible, and comprises materials that can significantly reduce the risk of thrombosis, compared to vascular grafts made from artificial polymers. Furthermore, the disclosed vascular graft has a vessel strength that is similar to a native vessel, which greatly increases the chance of success of surgical procedures and the post-surgical outcomes for patients.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments, Set 1

1. A method of producing a vascular graft including: selecting a group of cells; selecting an acellular tissue matrix and a hydrogel; and co-culturing the group of cells with the acellular tissue matrix and the hydrogel in a container; thereby producing the vascular graft.

2. The method of embodiment 1, wherein the hydrogel includes a structural protein and at least one component that facilitates the production of extracellular matrix proteins.

3. The method of embodiment 1 or 2, wherein the cells are fibroblasts.

4. The method of embodiment 3, wherein the fibroblasts are human dermal fibroblasts.

5. The method of any one of embodiments 1-4, wherein the cells are in a suspension prior to co-culture.

6. The method of embodiment 5, wherein the cells are trypsinized.

7. The method of any one of embodiments 1-6, wherein the acellular tissue matrix is a collagen-containing tissue matrix.

8. The method of embodiment 7, wherein the acellular tissue matrix is derived from dermal tissue.

9. The method of any one embodiments 1-8, wherein the acellular tissue matrix is completely decellularized.

10. The method of any one of embodiments 1-9, wherein the acellular tissue matrix is completely or substantially depleted of galactose alpha-1,3-galactose moieties.

11. The method of any one of embodiments 1-10, wherein the acellular tissue matrix is sterilized prior to use.

12. The method of any one of embodiments 2-11, wherein the structural protein in the hydrogel is fibrin.

14. The method of any one of embodiments 1-13, wherein the at least one component that facilitates the production of extracellular matrix proteins is ascorbic acid, transforming growth factor-beta (TGF-β), human insulin, human fibroblast growth factor (H-FGF), human epidermal growth factor (H-EGF), or a combination thereof.

15. The method of embodiment 14, wherein the hydrogel includes ascorbic acid and transforming growth factor-beta (TGF-β).

16. The method of embodiment 15, wherein the hydrogel includes about 0.1% to about 0.5% ascorbic acid and transforming growth factor-beta (TGF-β).

17. The method of embodiment 16, further including providing a supporting post in contact with the group of cells, the acellular tissue matrix, and the hydrogel.

18. The method of embodiment 17, wherein the supporting post is cylinder-shaped and has a dimeter of about 6 mm.

19. The method of any one of embodiments 1-18, further including cutting the acellular tissue matrix into a small piece prior to use.

20. The method of embodiment 19, wherein the small piece is ring-shaped with an inner diameter of about 6 mm, and an outer diameter of about 11 mm.

21. The method of embodiment 20, wherein the ring-shaped piece of acellular tissue matrix is positioned around the supporting post.

22. The method of any one of embodiments 1-21, wherein the hydrogel forms a layer on the bottom of the container, and the acellular matrix is positioned on top of the layer.

23. The method of any one of embodiments 1-21, wherein the acellular tissue matrix is positioned on the bottom of the container, and the hydrogel forms a layer on top of the acellular tissue matrix.

24. The method of any one of embodiments 1-23, wherein the cells self-assemble into a ring-shaped cellular structure and integrate into the acellular tissue matrix and hydrogel.

25. The method of embodiment 24, further including forming additional ring-shaped vascular grafts.

26. The method of embodiment 25, further including stacking the ring-shaped vascular grafts into a tubular, vascular-like structure.

27. A vascular graft produced by any one of embodiments 1-25.

28. A vascular graft including: a group of self-assembled cells; a collagen-containing tissue matrix; and a hydrogel including a structural protein and at least one component that facilitates the production of extracellular matrix proteins.

29. The vascular graft of embodiment 28, wherein the cells are fibroblasts.

30. The vascular graft of embodiment 29, wherein the cells are human dermal fibroblasts.

31. The vascular graft of embodiment 28, wherein the acellular tissue matrix is derived from dermal tissue.

32. The vascular graft of any one of embodiments 28-31, wherein the acellular tissue matrix is completely or substantially depleted of galactose alpha-1,3-galactose moieties.

33. The vascular graft of any one of embodiments 28-32, wherein the hydrogel includes fibrin.

34. The vascular graft of any one of embodiments 28-33, wherein the at least one component that facilitates the production of extracellular matrix proteins is ascorbic acid, transforming growth factor-beta (TGF-β), human insulin, human fibroblast growth factor (H-FGF), human epidermal growth factor (H-EGF), or a combination thereof.

35. The vascular graft of embodiment 34, wherein the hydrogel includes ascorbic acid and transforming growth factor-beta (TGF-β).

36. The vascular graft of any one of embodiments 28-35, wherein the graft is ring-shaped, with a diameter of about 6 mm.

37. The vascular graft of any one of embodiments 28-36, wherein the graft is formed by self-assembly of the cells.

38. The vascular graft of embodiment 37, further including at least one extracellular matrix protein synthesized by the vascular cells.

39. The vascular graft of embodiment 38, wherein the extracellular matrix protein is fibrillary collagen, collagen IV, elastin, hyaluronan, proteoglycans, fibronectin, or a combination thereof.

40. The vascular graft of embodiment 39, wherein the extracellular matrix protein is fibrillary collagen.

41. The vascular graft of any one of embodiments 28-40, wherein the cells, the collagen-containing tissue matrix and the hydrogel are integrated together.

42. A vascular graft including a tubular, vascular-like structure produced by embodiment 26.

43. A vascular graft of any one of embodiments 28-42, wherein the vascular graft is an adventitia graft.

### Exemplary Embodiments, Set 2

1. An all-natural method to support engineered tissues, essentially as described herein.

2. An all-natural means to provide support and mechanical strength to engineered tissues in order to fabricate all-natural, low immunogenicity tissues in vitro.

3. A bench-top model of atherosclerosis, essentially as described herein.

4. The bench-top model of embodiment 3, which is a tissue engineered model of atherosclerosis.

5. A bench-top model of atherosclerosis, essentially as described herein and which includes early and late state disease processes.

6. The bench-top model of any of embodiments 3-5, which model utilizes tissue engineered blood vessel (TEBV).

7. The bench-top model of embodiment 6, in which stages of atherosclerosis are induced in the TEBV by application of oxidized low-density lipoprotein (oxLDLs) (early stage), followed by macrophage introduction (early stage), and induction of calcification using calcified protein particles (CPPs; late stage).

8. The bench-top model of embodiment 6 or embodiment 7, wherein the TEBV is made by a method including: selecting a group of cells; selecting an acellular tissue matrix and a hydrogel; and co-culturing the group of cells with the acellular tissue matrix and the hydrogel in a container.

9. The bench-top model of embodiment 8, wherein the hydrogel includes a structural protein and at least one component that facilitates the production of extracellular matrix proteins.

10. The bench-top model of embodiment 8 or embodiment 8, wherein the cells are fibroblasts.

11. The bench-top model of embodiment 10, wherein the fibroblasts are human dermal fibroblasts.

12. The bench-top model of any one of embodiments 8-11, wherein the cells are in a suspension prior to co-culture.

13. The bench-top model of embodiment 12, wherein the cells are trypsinized.

14. The bench-top model of any one of embodiments 8-13, wherein the acellular tissue matrix is a collagen-containing tissue matrix.

15. The bench-top model of embodiment 14, wherein the acellular tissue matrix is derived from dermal tissue.

16. The bench-top model of any one embodiments 8-15, wherein the acellular tissue matrix is completely decellularized.

17. The bench-top model of any one of embodiments 8-16, wherein the acellular tissue matrix is completely or substantially depleted of galactose alpha-1,3-galactose moieties.

18. The bench-top model of any one of embodiments 8-17, wherein the acellular tissue matrix is sterilized prior to use.

19. The bench-top model of any one of embodiments 9-18, wherein the structural protein in the hydrogel is fibrin.

20. The bench-top model of embodiment 19, wherein the hydrogel includes thrombin and fibrinogen.

21. The bench-top model of any one of embodiments 8-20, wherein the at least one component that facilitates the production of extracellular matrix proteins is ascorbic acid, transforming growth factor-beta (TGF-β), human insulin, human fibroblast growth factor (H-FGF), human epidermal growth factor (H-EGF), or a combination thereof.

22. The bench-top model of embodiment 21, wherein the hydrogel includes ascorbic acid and transforming growth factor-beta (TGF-β).

23. The bench-top model of embodiment 22, wherein the hydrogel includes about 0.1% to about 0.5% ascorbic acid and transforming growth factor-beta (TGF-β).

24. The bench-top model of embodiment 3, further including providing a supporting post in contact with the group of cells, the acellular tissue matrix, and the hydrogel.

25. The bench-top model of embodiment 24, wherein the supporting post is cylinder-shaped and has a dimeter of about 6 mm.

26. The bench-top model of any one of embodiments 8-25, further including cutting the acellular tissue matrix into a small piece prior to use.

27. The bench-top model of embodiment 26, wherein the small piece is ring-shaped with an inner diameter of about 6 mm, and an outer diameter of 7-8 mm.

28. The bench-top model of embodiment 27, wherein the ring-shaped piece of acellular tissue matrix is positioned around the supporting post.

29. The bench-top model of any one of embodiments 8-28, wherein the hydrogel forms a layer on the bottom of the container, and the acellular matrix is positioned on top of the layer.

30. The bench-top model of any one of embodiments 8-28, wherein the acellular tissue matrix is positioned on the bottom of the container, and the hydrogel forms a layer on top of the acellular tissue matrix.

31. The bench-top model of any one of embodiments 8-30, wherein the cells self-assemble into a ring-shaped cellular structure and integrate into the acellular tissue matrix and hydrogel.

32. The bench-top model of embodiment 31, further including forming additional ring-shaped vascular grafts.

33. The bench-top model of embodiment 32, further including stacking the ring-shaped vascular grafts into a tubular, vascular-like structure.

34. A kit including the bench-top model of any one of embodiments 3-33.

35. Use of the kit of embodiment 34, to investigate disease processes and/or patient treatment options and/or drug development.

36. A method of making a benchtop model, essentially as described herein.

37. A method of inducing calcification in an in vitro atherosclerosis model, including introducing an effective amount of calcified protein particles (CPPs) into the model.

38. The bench-tope model of one of the preceding embodiments, in which stages of atherosclerosis are induced by application of oxidized low density lipoprotein (oxLDLs) (early stage), followed by macrophage introduction (early stage), and induction of calcification using calcified protein particles (CPPs; late stage).

39. The method of embodiment 1 or embodiment 2, which uses human cells and/or tissues, and has a substantially three-dimensional structure.

### Exemplary Embodiments, Set 3

1. An all-natural method to support engineered tissues, essentially as described herein.

2. An all-natural means to provide support and mechanical strength to engineered tissues in order to fabricate all-natural, low immunogenicity tissues in vitro.

3. A bench-top model of atherosclerosis (and/or an in vitro model of atherosclerosis), essentially as described herein.

4. The bench-top model of atherosclerosis of embodiment 3, which: is a tissue engineered model of atherosclerosis; and/or includes early and late state disease processes; and/or utilizes tissue engineered blood vessel (TEBV).

5. The bench-top model of embodiment 4, in which stages of atherosclerosis are induced in the TEBV by application of oxidized low-density lipoprotein (oxLDLs) (early stage), followed by macrophage introduction (early stage), and induction of calcification using calcified protein particles (CPPs; late stage).

6. The bench-top model of embodiment 4, wherein the TEBV is made by a method including: selecting a group of cells; selecting an acellular tissue matrix and a hydrogel; and co-culturing the group of cells with the acellular tissue matrix and the hydrogel in a container.

7. The bench-top model of embodiment 6, wherein one or more of: the hydrogel includes a structural protein and at least one component that facilitates the production of extracellular matrix proteins; and/or wherein the cells are fibroblasts; and/or the fibroblasts are human dermal fibroblasts; and/or the cells are in a suspension prior to co-culture; and/or the cells are trypsinized; and/or the acellular tissue matrix is a collagen-containing tissue matrix; and/or the acellular tissue matrix is derived from dermal tissue; and/or the acellular tissue matrix is completely decellularized; and/or the acellular tissue matrix is completely or substantially depleted of galactose alpha-1,3-galactose moieties; and/or the acellular tissue matrix is sterilized prior to use; and/or the structural protein in the hydrogel includes or is fibrin; and/or the hydrogel includes thrombin and fibrinogen.

8. The bench-top model of embodiment 7, wherein one or more of: the at least one component that facilitates the production of extracellular matrix proteins is ascorbic acid, transforming growth factor-beta (TGF-β), human insulin, human fibroblast growth factor (H-FGF), human epidermal growth factor (H-EGF), or a combination thereof; and/or the hydrogel includes ascorbic acid and transforming growth factor-beta (TGF-β); and/or the hydrogel includes about 0.1% to about 0.5% ascorbic acid and transforming growth factor-beta (TGF-β).

9. The bench-top model of embodiment 3, further including providing a supporting post in contact with the group of cells, the acellular tissue matrix, and the hydrogel.

10. The bench-top model of embodiment 9, wherein one or more of: the supporting post is cylinder-shaped and has a dimeter of about 6 mm; and/or further including cutting the acellular tissue matrix into a small piece prior to use; and/or the small piece is ring-shaped with an inner diameter of about 6 mm, and an outer diameter of 7-8 mm; and/or the ring-shaped piece of acellular tissue matrix is positioned around the supporting post; and/or the hydrogel forms a layer on the bottom of the container, and the acellular matrix is positioned on top of the layer; and/or the acellular tissue matrix is positioned on the bottom of the container, and the hydrogel forms a layer on top of the acellular tissue matrix; and/or the cells self-assemble into a ring-shaped cellular structure and integrate into the acellular tissue matrix and hydrogel; and/or further including forming additional ring-shaped vascular grafts; and/or further including stacking the ring-shaped vascular grafts into a tubular, vascular-like structure.

11. A kit including the bench-top model of embodiment 3, or another bench-top model described herein.

12. A method of using the kit of embodiment 11, to investigate a disease process and/or patient treatment option, and/or drug development.

13. A method of making the bench-top model of embodiment 3, or another bench-top model described herein.

14. A method of inducing calcification in an in vitro atherosclerosis model such as the one in embodiment 3, including introducing an effective amount of calcified protein particles (CPPs) into the model.

15. The method of embodiment 14, wherein the CPP are introduced into the model during its construction.

16. The bench-top in vitro model of embodiment 1, in which stages of atherosclerosis are induced by application of oxidized low density lipoprotein (oxLDLs) (early stage), followed by macrophage introduction (early stage), and induction of calcification using calcified protein particles (CPPs; late stage).

17. The method of embodiment, which uses human cells and/or tissues, and has a substantially three-dimensional structure.

### EXAMPLES

The following examples serve to illustrate, but in no way limit, the present disclosure.

### Example 1: Production of Vascular Grafts Using Acellular Dermal Matrix as a Scaffold

This example describes a representative method of making vascular grafts using acellular dermal matrix as a scaffold.

Cell Culture Human dermal fibroblasts (HuDF, PCS-201-012, ATCC, Manassas, Va.) were chosen as vascular cells to form grafts. Dermal fibroblasts are abundant and easy to procure and have advantages for future clinical application. Cell passages of 5-13 were used to ensure healthy morphology. Fibroblast growth medium (GM), consisting of 89% Dulbecco's Modified Eagle Medium (DMEM) high glucose solution, 10% fetal bovine serum, and 1% antibiotic/antimycotic, was used to expand and maintain the cell cultures. For expansion, cells were cultured in 150 mm petri dishes and incubated for 7 days at 37° C. and 5% CO2 to reach approximately 85% confluency. Differentiation medium (DM) consisted of 97% DMEM, 2% fetal bovine serum, and 1% antibiotic/antimycotic.

Assembly of Ring Formation Plates Poly(dimethylsiloxane) (PDMS) polymer (1064291, Dow Corning, Midland, Mich.) was used to coat 60 mm petri dishes, 3 mL per plate; and to fabricate and attach 6 mm posts to the center of each plate. Posts were cut out from blocks of PDMS using a biopsy punch and adhered to the plates after the PDMS coating had cured. Prior to seeding, plates were sterilized with a 30 min 70% ethanol soak and 30 min of UV sterilization under the bio-hood UV lamp.

Hydrogel Fibrin hydrogels were formed using thrombin, fibrinogen, and hydrogel medium. Specifically, a 4:1 ratio of 20 mg/mL bovine fibrinogen (151122, MP BIOMEDICALS LLC, OH) to 100 U/mL bovine plasma thrombin (7592, BIOVISION, Milpitas, Calif.) was used. Two different hydrogel medium were tested. Hydrogel medium growth factor high (GF-H) consisted of 88.5% 231 medium; 0.1% of recombinant human insulin (rH-insulin, 100-11, PEPROTECH, Rocky Hill, N.J.), recombinant human fibroblast growth factor (rH-FGF, 100-18, PeproTech), recombinant human epidermal growth factor (rH-EGF, 100-15, PEPROTECH), transforming growth factor-beta (TGF-β, 100-21, PEPROTECH), and ascorbic acid (A8960, SIGMA ALDRICH, St. Louis, Mo.); 5% of fetal bovine serum and L-glutamine; and 1% antibiotic-antimycotic. Hydrogel medium growth factor low (GF-L) consisted of 88.8% DMEM, 0.1% TGF-β and ascorbic acid, 10% fetal bovine serum, and 1% antibiotic/antimycotic.

Acellular Dermal Matrix Decellularized human skin dermis, i.e. ALLODERM® (Lifecell Corp., Madison, N.J.), was used as acellular tissue matrix to produce the vascular graft. Biopsy punches were used to cut 6 mm holes into decellularized dermis. A rotary cuter was used to cut a 2.5 mm width ring around the 6 mm hole. The final dimensions of the ring-shaped decellularized dermis encompassed an inner diameter of 6 mm and outer diameter of 11 mm. Dermis ring pieces were integrated into the engineered vascular grafts as described below. Prior to placement in the ring formation plate, dermis pieces were sterilized under UV light for 15 min on each side. Dermis pieces were kept in sterile saline until use. Additional dermis pieces were cut for mechanical testing to determine the dermis rings' mechanical properties alone.

Vascular Ring Formation Cultured fibroblasts were trypsinized from expansion plates and re-suspended in growth medium. Each 60 mm ring formation plate was seeded with 4 mL GM, 7.5×10⁵ cells, 150 μg ascorbic acid, and 0.01 ng TGF-β either onto fibrin gel directly or fibrin gel with dermis. Two different dermis placements were tried to determine the ideal step in which to insert the ECM—either below or on top of the fibrin hydrogel. FIG. 1 is a scheme illustrating one possible placement of the dermis and the fibrin hydrogel. The left panel shows the placement of each component at the beginning of the co-culture, and the right panel shows a hypothetical arrangement of each component in the ring-shaped vascular graft. Medium was changed one day after seeding and 6 days after seeding. The first medium change used growth medium, whereas the second medium change used differentiation medium. Each medium change was supplemented with the same concentration of ascorbic acid and TGF-β.

Integration of Cellular Components into Acellular Dermal Matrix

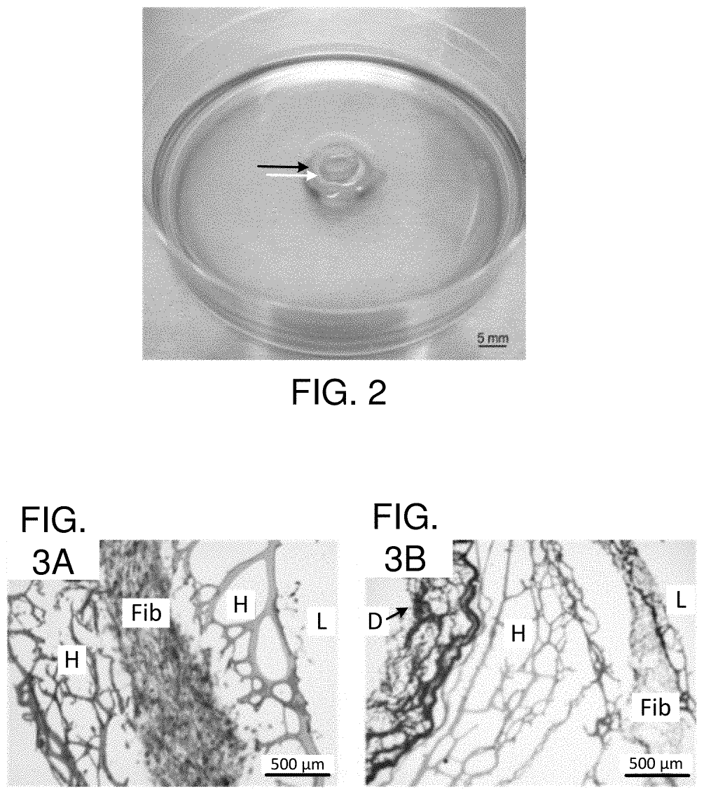

As shown in FIG. 2, vascular rings were formed by self-assembly of the fibroblasts and the integration of all components in the co-culture. The hydrogel (black arrow) enwraps the dermis (white arrow) and cells. The post diameter is 6 mm, equating to the adventitia ring lumen diameter.

Rings with and without dermis were stained with Masson's Trichrome staining to visualize the internal composition of the rings and location of the dermis. In adventitia rings without dermis, a band of fibroblasts (“Fib”) between layers of hydrogel (“H”) was evident (FIG. 3A). Collagen was observed intertwined with the cells. In the dermis containing rings, the dermis (“D”) was apparent and resides on the outer edge of the ring opposite of the lumen (“L”) (FIG. 3B).

In rings produced with dermis, some degree of disconnection was observed in the histology results between the cells and the hydrogel component, which is attributed to the frozen sectioning process. Hydrogel (“H”) was observed lining the fibroblast cells in the dermis rings, similar to in the non-dermis rings.

### Acellular Dermal Matrix Provides Structural Support for the Vascular Graft and Increases Vessel Strength

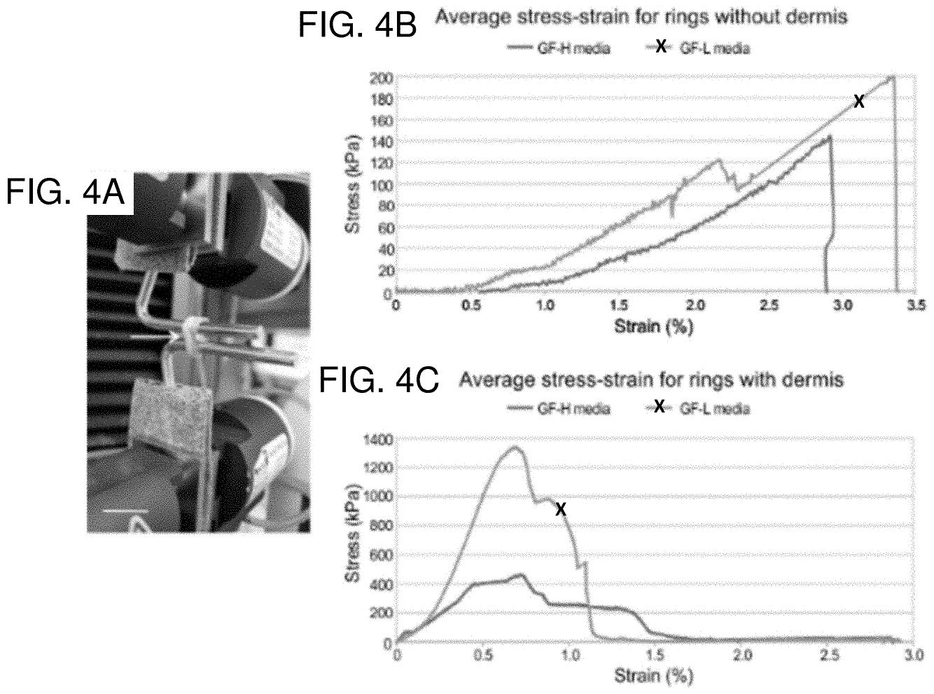

Tensile testing was performed using a UStretch mechanical testing system with a 5 N load cell (CELLSCALE, Waterloo, Ontario, Canada) for non-dermis containing rings and an INSTRON 5943 with a 50 N load cell (INSTRON, Norwood, Mass.) for dermis containing rings (FIG. 4A). Strain rate was kept constant in order for results from both mechanical testers to be compared. Ring-shaped vascular graft and acellular dermis piece alone were circumferentially stretched until failure at a strain rate of 0.4 mm/min. Vascular graft were mechanically tested 9 days after seeding. Stress-strain curves were obtained from the tensile data, and the elastic modulus, ultimate tensile strength, and failure strength were determined from the curves. Vascular grafts with dermis with different hydrogel medium compositions were tested to determine which combination of hydrogel and dermis lead to the highest strength in the engineered vascular rings. The ring-shaped vascular graft produced with decellularized dermis were stretched using INSTRON due to its higher strength and need for a larger load cell. Sample number for rings with dermis with different hydrogel mediums were: growth factor high (GF-H) hydrogel medium (n=6), and growth factor low (GF-L) hydrogel medium (n=5). Sample number for rings without decellularized dermis with different hydrogel mediums were: GF-H hydrogel medium (n=4), and GF-L hydrogel medium (n=5). Vascular grafts without decellularized dermis were stretched using the UStretch system. Decellularized dermis rings were tensile tested alone to determine their contribution to tensile strength in the tissue rings. Decellularized dermis donuts sterilized (n=6) and unsterilized (n=6) were stretched using the INSTRON.

Mechanical properties of vascular grafts and dermis piece alone were determined from stress-strain curves. Elastic moduli (E), ultimate tensile strength (UTS), and failure strength (FS) were obtained for vascular grafts with and without decellularized dermis with different hydrogel mediums and dermis pieces (Table 1). Vascular grafts with decellularized dermis with varying hydrogel medium had a statistically significance difference between elastic moduli (p<0.01) and ultimate tensile strength (p≤0.001), indicating a difference in material properties due to the hydrogel medium. Average stress-strain curves for all vascular grafts with decellularized dermis are shown in Table 1.

TABLE 1
Tensile Properties of Rings and Decellularized Dermis Rings

| | | Tensile Properties (kPa) | | |
|---|---|---|---|---|
| Decellularized | Hydrogel | Elastic | Ultimate Tensile | Failure |
| Dermis Groups | Media | Modulus (E) | Strength (UTS) | Strength (FS) |
| Rings without | GF-H | 79.7 ± 6.85 ^{b} | 144 ± 20.6 ^{b} | 73.5 ± 42.9 ^{b} |
| decellularized dermis | GF-L | 86.1 ± 9.36 ^{c} | 173 ± 21.5 | 109 ± 23.9 |
| Rings with | GF-H | 1830 ± 163 ^{a, b, d} | 656 ± 53.4 ^{a, b, d} | 171 ± 72.3 ^{b} |
| decellularized dermis | GF-L | 3480 ± 1130 ^{a, c, e} | 1400 ± 349 ^{a, c, e} | 101 ± 13.0 ^{e} |

| | | | |
|---|---|---|---|
| Decellularized dermis donut alone | 12300 ± 1830 ^{d, e} | 4720 ± 630 ^{d, e} | 223 ± 37.8 ^{e} |

^{a} denotes statistical significance between decellularized dermis rings with different hydrogel (E: p < 0.01; UTS: p ≤ 0.001).
^{b} denotes statistical significance between rings with hydrogel one with or without decellularized dermis (E: p < 0.001; UTS: p < 0.001; FS: p < 0.05).
^{c} denotes statistical significance between rings with hydrogel two with or without decellularized dermis (E: p < 0.001; UTS: p < 0.001)
^{d} denotes statistical significance between decellularized dermis donut and rings with hydrogel medium one with decellularized dermis (E: p < 0.001; UTS: p < 0.001)
^{e} denotes statistical significance between decellularized dermis donut and rings with hydrogel medium two with decellularized dermis (E: p < 0.001; UTS: p < 0.001; FS: p < 0.001)

Average stress-strain curves for all vascular grafts without decellularized dermis are shown in FIG. 4B. Rings without decellularized dermis and fabricated with GF-H hydrogel medium had an average E, UTS, and FS of 79.7±6.85 kPa, 144±20.6 kPa, and 73.5±42.9 kPa, respectively. Comparatively, vascular grafts without decellularized dermis and GF-L hydrogel medium had higher average values for E, UTS, and FS, although not significantly, equating to 86.1±9.36 kPa, 173±21.5 kPa, and 109±23.9 kPa, respectively.

Average stress-strain curves for all vascular grafts produced with decellularized dermis are shown in FIG. 4C. Vascular grafts with decellularized dermis and fabricated with GF-H hydrogel medium had an average E, UTS, and FS of 1830±163 kPa, 656±53.4 kPa, and 171±72.3 kPa, respectively. Vascular grafts without decellularized dermis and GF-L hydrogel medium had significantly higher average values for E, UTS, and FS, equating to 3480±1130 kPa, 1400±349 kPa, and 101±13.0 kPa, respectively.

When comparing rings with and without decellularized dermis while keeping hydrogel medium constant, statistical significance was observed, indicating improved material properties due to the inclusion of the decellularized dermis. Vascular grafts constructed with GF-H hydrogel medium with decellularized dermis exhibited significantly increased E (p<0.001), UTS (p<0.001), and FS (p<0.05) compared to vascular grafts with GF-H hydrogel medium without decellularized dermis. Vascular grafts constructed with GF-L hydrogel medium with decellularized dermis exhibited significantly increased E (p<0.001), and UTS (p<0.001) compared to vascular grafts with GF-L hydrogel medium without decellularized dermis.

Decellularized dermis vascular grafts alone had an average E, UTS, and FS of 12,300±1830 kPa, 4,720±630 kPa, and 223±37.8 kPa, respectively. Statistical significance was observed when comparing material properties of dermis rings and rings with dermis rings in each hydrogel medium, indicating a difference in material properties due to inclusion of the cells and hydrogel in the rings. Decellularized dermis pieces alone compared to rings with dermis and GF-H hydrogel medium showed a statistically significant difference in values for E (p<0.001) and UTS (p<0.001). Decellularized dermis donuts alone compared to rings with dermis and GF-L hydrogel medium showed a statistically significant difference in E (p<0.001), UTS (p<0.001), and FS (p<0.001).

### Example 2: Adipose-Derived Stem Cells Significantly Increases Collagen Level and Fiber Maturity in Patient-Specific Biological Engineered Blood Vessels

This Example demonstrates efficacy of ASCs and PtFibs to generate patient-specific vessels.

Identifying a highly-sought after autologous cell source for tissue engineering remains elusive. Here, efficacy of patient-sourced cells derived from adipose (adipose-derived stem cells, ASCs) and skin tissue (dermal fibroblasts, PtFibs) is presented to build an engineered tunica media and adventitia, respectively. Patient cells were implemented into the lab's vascular tissue engineering techniques of forming vascular rings that are stacked into a tubular structure. ASCs were successfully differentiated into the smooth muscle phenotype using angiotensin II followed by culture in smooth muscle growth factors, evidenced by significantly increased expression of αSMA and myosin light chain kinase. Differentiated ASCs (ASC-SMCs) exhibited an elastic modulus (75.0±37.4 kPa) between that of undifferentiated ASCs (118±75.9 kPa) and control human aortic smooth muscle cells (HASMCs; 28.3±8.69 kPa) (p<0.5). ASC (41.3±15.7 kPa) and ASC-SMC (37.3±17.0 kPa) vessels exhibited higher tensile strength compared to vessels of HASMCs (28.4±11.2 kPa). MYLK expression in ASC-SMCs was significantly higher relative to undifferentiated ASCs and comparable to HASMCs (p<0.05). ASC-based tissues exhibited a significant increase in collagen content and fiber maturity as shown by polarized light.

### INTRODUCTION

For vascular repair, the gold standard autograft is at critically low supply (Hess et al., Circulation 130:1445-1451, 2014; Kimicata et al., Tissue Eng Part A 26:1388-1401, 2020). Synthetic grafts composed of polymers have shown a high incidence of thrombogenicity and cases of infection (Hoshi et al., Biomaterials 34:30-41, 2013; Kirkton et al., J Surg Res 221:143-151, 2018). A completely biological engineered vascular graft option would offer a superior solution. Fortunately, tissue engineering of vascular grafts has been a steadily developing field.

Current vascular tissue engineering methods have focused on developing tubular structures with vascular-like cells from allogenic or xenogenic origins incorporated to induce extracellular matrix deposition and remodeling. Though these cells types are useful for proof-of-concept, both allogenic and xenogenic cells are difficult to translate into an implant due to immunogenicity issues and rejection (Benedetto et al., J Vasc Surg 34:139-142, 2001). The current solution for allogenic cell utilization is decellularization of grafts to eliminate cellular immunogenic components (Lawson et al., Lancet 387:2026-2034, 2016; Syedain et al., Sci Transl Med 9, doi:10.1126/scitranslmed.aan4209, 2017; Wystrychowski et al., J Vasc Surg 60:1353-1357, 2014). However, direct decellularization of grafts can cause issues of graft viability and eliminate cellular functions controlling vital vascular mechanisms (Van de Walle et al., Cardiovasc Eng Technol 6:303-313, 2015). In order for a decellularized graft to achieve functionality in vivo, endogenous cell recellularization of the graft is needed (Syedain et al., Sci Transl Med 9, doi:10.1126/scitranslmed.aan4209, 2017). Recently, a meta-analysis of small-diameter tissue engineered grafts revealed that longer recellularization rates significantly negatively affected long-term patency and graft survival (Skovrind et al., Stem Cells Transl Med 8:671-680, 2019).

Viable autologous cell sources would solve the current major issues hindering application of vascular engineered tissues to the clinic. Specifically, an autologous source for smooth muscle cells (SMCs) for the tunica media and fibroblasts for the tunica adventitia are needed to provide the functional and structural components of the vascular wall in a graft.

Presently, an autologous source of vascular SMCs is not available since sacrificial arteries from a patient are not readily available for SMC harvest. Stem cells are a potential source for vascular SMCs. Adult stem cells would be able to be harvested from a patient to provide autologous cells, depending on the tissue of origin. Beneficially, adipose-derived stem cells (ASCs) are abundant and easily harvested from patient adipose tissue obtained through either liposuction or lipectomy. In addition, ASCs derive from the same mesenchymal germ layer as SMCs, suggesting that differentiation of ASCs to an SMC phenotype will more likely be successful due to their similar lineages.

Advantageously, ASCs have demonstrated the ability to express SMC contractile proteins in vitro following treatment with different growth factors (Kim et al., Int J Biochem Cell Biol 40:2482-2491, 2008; Lau et al., Tissue Eng Part A 25:936-948, 2019; Wang et al., Tissue Eng Part A 16, 1201-1213, 2010). ASCs have been shown to improve the hemocompatibility of decellularized and synthetic polymer grafts, including demonstration of improved short-term patency and expression of smooth muscle contractile proteins in ASC-seeded poly(ester urethane)urea grafts (Krawiec et al., J Vasc Surg 66, 883-890 e881, 2017; Krawiec et al., Tissue Eng Part A 22, 765-775, 2016; La & Tranquillo, Tissue Eng Part A 24, 1242-1250, 2018). Utilization of findings such as these incorporated with further development of ASC application into patient-specific vascular grafts is needed to achieve clinical use.

The main role of fibroblasts in the adventitia is to produce and deposit collagen to provide strength and structural integrity to the blood vessels (Stenmark et al., Annu Rev Physiol 75, 23-47, 2013). Skin is a viable means to harvest autologous dermal fibroblasts (Vangipuram et al., J Vis Exp, e3779, 2013). Dermal fibroblasts have been shown to extensively promote collagen deposition in tissue-engineered grafts (Gui et al., Tissue Eng Part A 20, 1499-1507, 2014; L'Heureux et al., Nat Med 12, 361-365, 2006; Syedain et al., Biomaterials 32, 714-722, 2011), thus aiding in extracellular matrix development and tissue strength. While recent single-cell RNA sequencing analyses suggest some unique heterogeneity among fibroblasts from different anatomical regions, genes related to wound healing and tissue remodeling, such as collagen type 1 alpha 1 chain, remain conserved (He et al., Genome Biol 21, 294, 2020). Additionally, adventitial and dermal fibroblasts have demonstrated similar responses to substrate stiffness through upregulating alpha smooth muscle actin and collagen types 1 and 3 alpha 1 chains (Achterberg et al., J Invest Dermatol 134, 1862-1872, 2014; Wang et al., Cells 10, doi:10.3390/cells10051000, 2021). Applicability of dermal fibroblasts to our engineered adventitia was evaluated based on collagen levels and maturity of collagen fibers.

Here, previously reported methods (Pinnock et al., Methods 99:20-27, 2016; Pinnock et al., J. Vis. Exp. 55322, 2017; Patel et al., Sci. Rep 8, 3294, 2018) were utilized for tissue engineering blood vessels, called the Ring Stacking Method (RSM), for assessing applicability of autologous patient cells of ASCs and dermal fibroblasts to create a patient-specific vascular graft. A source for patient endothelial cells (ECs) was previously explored, using ASCs as the base cell. Several endothelial growth factor protocols were tried with little success for differentiation towards the endothelial cell phenotype. In addition, there is no source of harvestable adult autologous endothelial cells. Advantageously, implanted tubular structures, such as stents, are endothelialized in the body, hence establishment of a native intima is an inherent process (Kirkton et al., Sci Transl Med 11, 2019; Koobatian et al., Biomaterials 76, 344-358, 2016), allowing for potential establishment of the intima in a media-adventitia graft.

In this example, “patient” cells were derived from full-thickness skin samples from patients undergoing elective abdominoplasty. ASCs were isolated from the adipose tissue of the hypodermis, then differentiation towards the SMC phenotype was explored for building the tunica media. Undifferentiated ASCs and human aortic SMCs (HASMCs) served as controls. Patient fibroblasts (PtFibs) were extracted from the dermal layer of the skin samples through explant cultures. Both ASC-SMC and PtFibs were successfully implemented into the established engineered vessel protocol. Differentiation of ASCs into SMCs (ASC-SMCs) using angiotensin II and prolonged culture in smooth muscle growth factors promoted expression of smooth muscle contractile proteins, showing successful differentiation. Histology revealed that both ASC-based vascular tissues produced significantly more collagen than tissues consisting of human aortic smooth muscle cells controls, showing that ASCs have a higher capacity for collagen production and deposition than HASMCs. The comparatively higher collagen production by ASCs resulted in higher mechanical strength in ASC-based vessels compared to HASMC control vessels as shown by tensile tests. Interestingly, PtFib tissues exhibited limited collagen deposition and lowest tensile strength compared to SMC and ASC-based engineered vessels. Polarized light analysis revealed increased maturity of collagen fibers in the ASC-SMC tissues compared to those in HASMC tissues. One month culture of vessels showed increased cellularity and collagen deposition. Combined “bilayers” of ASC-SMC tunica media with PtFib tunica adventitia were successfully formed, thus generating a potential all-biological engineered graft.

This Example shows that ASCs and dermal fibroblasts are viable patient cell source options for vascular tissue engineering.

### Methods

Cell Isolation and Culture. Human abdominal skin and adipose tissues were obtained with informed patient consent from elective abdominoplasty surgeries at Henry Ford Hospital—Main (Detroit, Mich.) and Henry Ford Medical Center—Cottage (Grosse Pointe Farms, Mich.) in accordance with Wayne State University and Henry Ford Health System Institutional Review Board (IRB) guidelines. Full thickness skin was collected from patients. The dermis was used for fibroblast isolation and the hypodermis was used for ASC harvest. Excised tissues were placed on ice and cells extracted within 24 hours post-operation.

ASCs were extracted by mechanical digestion of adipose tissue using scalpels followed by enzymatic digestion in 1 mg/mL collagenase type II. The cellular component was separated from the extract mixture by centrifugation at 1000 rpm. Following aspiration of the supernatant, the resultant pellet containing the stromal vascular fraction was resuspended in basic growth media consisting of 89% Dulbecco Modified Eagle Medium (DMEM), 10% fetal bovine serum (FBS), and 1% antibiotic-antimycotic. The stromal vascular fraction containing ASCs, was expanded in culture plates. ASC cultures were used in the experiments between passages 3-6 to maintain ASC viability (Mitchell et al., Stem Cells 24, 376-385, 2006).

PtFibs were extracted through explant culture. Following ASC isolation, full thickness skin was cleared of remaining adipose tissue and cut into 3×3 mm sections. Skin sections were placed on 10% gelatin-coated culture dishes containing basic growth media with the dermis in contact with the gelatin surface. PtFibs were observed migrating from the excised skin sections onto the culture plate surface after 1-2 weeks. Skin sections were removed from the culture and extracted fibroblasts were expanded in 150 mm petri dishes and used for experiments at passages 4-10 to maintain viability.

Human aortic smooth muscle cells (HASMC; PCS-100-012, ATCC, Manassas, Va.) were used to generate the positive controls for the smooth muscle differentiation assays and tissue engineered tunica media. Smooth muscle growth media containing 88.5% DMEM; 5% of L-glutamine and fetal bovine serum; 1% antibiotic-antimycotic; 0.1% of recombinant human insulin (rH-insulin), recombinant human epidermal growth factor (rH-EGF), recombinant human fibroblast growth factor (rH-FGF) and ascorbic acid was used for HASMC expansion. Cells were utilized for experiments between passages 3-10.

Human femoral artery. Fully intact human femoral arteries were harvested from fresh, non-treated cadavers donated to the Wayne State University Body Bequest Program. Arteries were tensile tested to determine mechanical properties and histologically processed to assess tissue morphology to serve as a control.

ASC Differentiation. Angiotensin II was chosen as a demonstrated myogenic differentiation factor for ASCs, as it is known to upregulate smooth muscle contractile protein expression (Kim et al., Int J Biochem Cell Biol 40:2482-2491, 2008). ASC passages 3-4 were seeded at a density of 2×10³ cells/cm² in a differentiation media containing low glucose DMEM supplemented with 4% FBS, 1% antibiotic-antimycotic, and 2 μM angiotensin II (AngII). Two differentiation protocols were investigated: 1) ASC differentiation for 7 days in angiotensin II (group labeled “ASC-SMC I”); and 2) ASCs differentiated for 7 days in angiotensin II differentiation media, followed by culture for 7 days in smooth muscle growth media with smooth muscle growth factors of insulin, EGF, and FGF (group labeled “ASC-SMC II”). Media was changed in both groups every 2 days to maintain consistent differentiation factor exposure. Differentiated groups were analyzed for smooth muscle myogenic contractile marker expression using qRT-PCR analysis and for functionally through a gel contraction assay.

Preparation of Engineered Vascular Tissue Culture Plates. The engineered vascular constructs are fabricated using our previously established methods whereby vascular cell monolayers temporarily supported by a fibrin hydrogel are self-assembled into tissue rings which are then stacked to from the final vessels' tubular structure (Patel et al., Sci Rep 11, 11384, 2021; Patel et al., Sci Rep 8, 3294, 2018; Pinnock et al., Methods 99, 20-27, 2016; Pinnock et al., J Vis Exp, doi:10.3791/55322, 2017). To fabricate the ring plates, 6-well culture dishes were thinly surface coated with a 10:1 base to curing reagent mixture of poly(dimethysiloxane) elastomer (PDMS). Once polymerized, a 6 mm diameter cylindrical PDMS post was adhered to the center of each well with additional PDMS. The hydrophobic surface created by the PMDS allowed for detachment of the cell monolayer once formed, followed by self-aggregation of the cell monolayer around the post placed in the middle of the custom plate, thus forming the ring structure.

To fabricate the vessel plates, cylindrical poly(carbonate) tubing was attached to a poly(carbonate) base with acrylic glue. Poly(lactic) acid (PLA) removable posts with diameters of 6 mm and length of 15 mm were 3D-printed. Posts were lightly filed to remove 3D printing burrs, then thinly coated with PDMS to protect the engineered vascular tissues. Post holders were attached to the center of the vessel dishes and posts were fit into the holder allowing for subsequent removal of the vessel. Both ring and vessel plates were sterilized first with a 30 min ethanol soak followed by 30 min of UV prior to use.

Engineered Vascular Tissue Fabrication—Rings. Single-cell type (i.e. single layer) and two-cell type (i.e. bilayer) vascular tissue rings were created by seeding fibroblasts or fibroblasts and smooth muscle cells onto fibrin hydrogel in the ring plates with the central post for ring formation. Briefly, the rings formed first by proliferation of the seeded cells into a cell monolayer. Attachment and detachment of the cell monolayer to the bottom of the plate was controlled by the silicone elastomer deposited on the bottom of the plate allowing for tissue self-organization, causing the monolayer to detach from the bottom once formed. Lastly, the monolayer aggregated towards the center and wraps around the post to form the vascular ring structure (Patel et al., Sci Rep 11, 11384, 2021; Patel et al., Sci Rep 8, 3294, 2018; Pinnock et al., Methods 99, 20-27, 2016; Pinnock et al., J Vis Exp, doi:10.3791/55322, 2017). The rings are then stacked into the final vessel form (FIG. 5). Each cell type was tested separately by creating rings of ASCs (control), ASC-SMCs I, ASC-SMCs II, HASMCs (control), or PtFibs. Cells prepared for seeding were suspended in 20 mg/mL bovine plasma fibrinogen for cells seeded inside the fibrin gels, or in ring culture media for seeding on top of the fibrin gels. Fibrin hydrogels were fabricated in the ring culture plates by depositing 0.5 mL of media composed of either SMC growth media for HASMC and ASC-based rings, or general growth media for PtFib rings. Next, 40 μL of 100 U/mL bovine plasma thrombin and 160 μL of fibrinogen containing 1×10⁶ cells were added to each well to create the fibrin hydrogel. Following fibrin gel polymerization, an additional 1×10⁶ cells suspended in their respective culture media, SMC growth media with 0.05 ng/mL transforming growth factor beta-1 (TGF-β1) for HASMC and ASC-based rings, or general growth media with 0.025 ng/mL TGF-β1 and 37.5 μL ascorbic acid for PtFib rings, were added dropwise on top of each gel. Plates were gently swirled to ensure uniform cell distribution followed by overnight culture in an incubator. After overnight incubation, media was changed followed by every 48 h thereafter for 7 days.

Combined tunica media and tunica adventitia “bilayer” rings were generated by plating adventitia rings around completed media rings. Media rings consisted of either ASC-SMC or HASMC cells depending on the group. For bilayer rings containing patient-derived cells, ASCs and PtFibs from the same patient (i.e. “patient-matched” cells) were used in the same bilayer rings to minimize effects from patient variability in the engineered tissues. Bilayer rings were constructed by first transferring fully formed ASC-SMC or HASMC rings on day 7 after initial cell seeding with sterilized forceps onto the PDMS posts of new ring plates into 0.5 mL of media of general growth media with PtFib cells for forming the adventitia ring. Rings were submersed in the PtFib cell media to coat the media rings which aided in the attachment between the engineered tunica media and adventitia. Next, PtFibs were seeded inside and on top of the secondary hydrogel. The PtFibs-hydrogel combination then aggregated towards the center of the plate to form an adventitia ring around the media ring, thus completing the bilayer ring.

Engineered Vascular Tissue Fabrication—Vessels. Single and bilayer vessels were created using our lab's previously published Ring Stacking Method (RSM) in which engineered vascular rings are stacked and adhered together with fibrin glue to form a vascular tube. Briefly, following ring formation, depending on the group, 3 or 6 rings were transferred from their ring plates to a 6 mm diameter 3D printed PLA post within a custom vessel culture dish. Ring in the stacks were placed in contact with each other with sterile forceps to limit gaps and the stack coated with additional fibrin gel in a 1:1 volumetric ratio of 100 U/mL thrombin and 20 mg/mL fibrinogen. Once the fibrin coating polymerized, vessels were cultured in their respective growth medias for 2 days prior to mechanical testing or for 4 weeks for long-term histological analysis.

Quantitative Reverse Transcription Polymerase Chain Reaction (qRT-PCR). Expression profiles of mesenchymal (endoglin, CD105; leukocyte common antigen, CD45; and thymocyte differentiation antigen-1, CD90) and smooth muscle (transgelin, TAGLN; smooth muscle alpha-2 actin, ACTA2; smoothelin, SMTN; and calponin-1, CNN1) gene markers were acquired via qRT-PCR. GAPDH served as the housekeeping gene. Total RNA was extracted from cell lysates of cultured HASMCs (n=5) and ASC groups (n=5 patients) using an RNA purification GeneJET kit (FERK0731, ThermoFisher Scientific). Sample RNA concentrations were measured with a Qubit 2.0 Fluorometer and 1 μg of RNA from each sample was reverse transcribed with TaqMan Reverse Transcription Reagents kit (N8080234, Applied Biosystems Inc.). Quantitative PCR was performed in triplicates using 20 μL volumes consisting of PowerUp SYBR Green PCR master mix, PCR primers, and cDNA in a StepOne Plus Real-Time PCR system. Results were analyzed using the 2^{-ΔΔCt} method and presented as fold-change with respect to undifferentiated ASCs (Taylor et al., Trends Biotechnol 37, 761-774, 2019).

Immunofluorescence Imaging. Cellular production of smooth muscle contractile proteins alpha-smooth muscle actinin (αSMA) and myosin light chain kinase (MYLK) were analyzed in 2D cell cultures to evaluate differentiation protocols and in engineered vascular tissues to assess phenotype. For cell cultures, 30×10³ cells were seeded onto 0.1% gelatin coated glass slide covers and placed in PDMS coated culture wells. ASCs and HASMCs served as negative and positive controls for SMC differentiation, respectively. After 2 days of incubation, cells were fixed with 5% formalin. Cells were permeabilized with 0.1% Triton X-100 in TBS-Tween20 and blocked with TBS-Tween20 containing 3% bovine serum albumin. Mouse αSMA antibodies and Rabbit MYLK Polyclonal antibodies were diluted to concentrations of 4 μg/mL and 10 μg/mL, respectively, and used to incubate cells overnight at 4° C. in a wet box. The mouse αSMA monoclonal antibody developed by Little, C.D. was obtained from the Developmental Studies Hybridoma Bank, created by the NICHD of the NIH and maintained at The University of Iowa, Department of Biology, Iowa City, Iowa 52242. Coverslips were washed with PBS and incubated for 1 h with 10 μg/mL goat anti-mouse IgG cross-adsorbed secondary antibody Alexa Fluor 488 and 15 μg/mL goat anti-rabbit IgG cross-adsorbed secondary antibody Alexa Fluor 555 in PBS containing 1% bovine serum albumin. Finally, coverslips were washed with TBS-Tween20 and mounted with Fluoromount-G mounting medium containing DAPI.

Engineered tissues and the femoral artery control were fixed in 10% formalin followed by dehydration in alcohol and xylene. Dehydrated samples were mounted in paraffin and sectioned cross-sectionally at 7 μm thicknesses. Slides were cleared in xylene and rehydrated using a decreasing gradient of alcohol solutions and water. Prepared slides were immunofluorescent stained for αSMA antibody and DAPI.

Fluorescent images were acquired using an EVOS FL inverted fluorescent microscope and analyzed via ImageJ. Intensity quantification of 2D cultured cells was achieved by tracing non-overlapping individual cells and measuring their area and integrated density at each fluorescent channel of interest. The intensities were normalized by the average of three background intensities for each image and presented as intensity per cell area. Images were batch edited for brightness to ensure consistency in minimal processing.

Fibrin Gel Contraction Assay. Cell contractility assays were conducted as a functional measure of smooth muscle differentiation. Total area of gels seeded with the SMC-differentiated cells were measured over time, with decreasing size of the gel correlating to higher cell contractility (Lau et al., Tissue Eng Part A 25:936-948, 2019). Fibrin hydrogels were seeded with either ASC-SMCs, undifferentiated ASCs (control), or HASMCs (control) with n=6 gels per group. Hydrogel change in size was captured with time-lapse imaging over a 24 h period. Hydrogels were formed in 6-well plates by mixing 0.5 mL general cell growth media, 40 μL of 100 U/mL thrombin and 160 μL/mL of 20 mg/mL fibrinogen containing 500×10³ cells/mL. Following gel formation, 2 mL of smooth muscle growth media containing an additional 500×10³ cells/mL was carefully deposited dropwise on top of the fibrin hydrogel. Images of each gel were acquired immediately after seeding at 0 h and every hour for 6 h, and at 12, 21, and 24 h time points. Overall surface area of each gel were determined at each time point by outlining the gel perimeter in ImageJ followed by calculating the area in pixels and converting to cm² by using a reference scale in each image.

Mechanical Analysis of Engineered Tissues. Mechanical properties of single and bilayer engineered vascular rings and vessels were assessed with circumferential tensile testing using a UStretch mechanical testing system (CellScale, Waterloo, Ontario, Canada) equipped with 5 N load cell. Tissue rings (n=15-22 per group) were tested on day 7 of culture, while vessels (n=5-6 per group) were tested 2 days post-stacking. Uniaxial circumferential tensile testing was performed at a strain rate of 0.4 mm/min until failure. Specimens were attached to the system by connecting metal hooks to the system clamps and inserting the hook through the engineered tissue lumen. Samples were placed at slack length and measurements for thickness and width were obtained using digital calipers at two points to determine cross-sectional area for stress calculations. Once stretched to failure, the elastic modulus (E), ultimate tensile strength (UTS), failure strength (FS), and percent elongation at failure were determined through stress-strain curve analysis.

Tissue Histology. The organization of cellular and extracellular matrix components of engineered vascular rings, long-term cultured vessels, and fresh non-preserved cadaver femoral arteries (control) embedded in paraffin were analyzed histologically. Samples were exposed to 10% formalin for 24 h at room temperature followed by dehydration and temporary storage in 70% EtOH at 4° C. Tissues were processed through a gradual 70 to 100% ethanol dehydration over 12 h followed by xylene. Next, samples were exposed to paraffin wax at 60° C. for 2 h, embedded into paraffin blocks, and sectioned at 7 μm thickness. Hematoxylin and eosin (H&E) staining was performed to determine overall cellularity, cellular alignment, the degree of extracellular matrix deposition, and organization of collagen. Collagen content in the samples was assessed using Masson's trichrome (collagen marked blue) and Picrosirius red (collagen marked red) stains. Additionally, visualizing Picrosirius red stains under polarized light was used to differentiate thinner, immature collagen fibers (green to yellow) from dense, mature fibers (orange to red). Picrosirius red images were further analyzed using ImageJ to quantify the total amount of collagen fibers per engineered tissue area. Verhoeff Van-Gieson (VVG) staining was performed to assess elastin fiber (black) content in tissue sections.

Statistics. Immunofluorescence quantification, hydrogel contraction, vascular tissue mechanics, and collagen quantification data were presented as mean values±standard deviation. PCR data was presented as the mean value with confidence intervals derived from the standard error (Taylor et al., Trends Biotechnol 37, 761-774, 2019). One-way ANOVA with a Tukey's-b post-hoc test and an alpha value of 0.05 was used to determine statistical significance between groups in biochemical, mechanical, and collagen quantification datasets. Statistical significance for 1-month vessel collagen quantification was determined by an independent t-test with an alpha value of 0.05. Statistical analyses were calculated using SPSS.

### Results

Angiotensin II with SMC Growth Factors Induces Smooth Muscle Phenotype in ASCs. Previously, our lab demonstrated the ability to isolate CD105⁺/CD90⁺ mesenchymal stem cells from human adipose tissues (Meier et al., Plast Reconstr Surg Glob Open 4, e864, 2016). Relative gene expression of CD105⁺, CD90⁺ and CD45⁻ were analyzed through qRT-PCR (FIG. 6A). Average expression of CD105⁺ and CD90⁺ were slightly higher in ASC groups compared to HASMCs, though not significantly. CD45⁻ expression did not differ significantly between groups, however, the cycle threshold values obtained for CD45 were near the limit of detection, suggesting expression was limited in all groups examined. To determine the effect of additional SMC growth factor supplementation from the SMC media on differentiation, in the ASC-SMC II group, qRT-PCR for smooth muscle contractility proteins TAGLN, ACTA2, SMTN, and CNN1 was performed (FIG. 6B). Interestingly, HASMC expression of TAGLN, ACTA2, and CNN1 was found to be lower, but not significantly, compared to the ASC-SMC I and ASC-SMC II cells. Similarly, AngII+SMC-GM cultured ASCs had nonsignificant, lower expression of TAGLN and ACTA2, and significantly lower CNN1 expression relative to undifferentiated and AngII treated ASCs (p<0.05). Yet, HASMCs and ASC-SMC II cells showed increased expression of SMTN compared to ASCs and AngII stimulated ASCs, though not significantly.

The effect of culture conditions on ASCs contractile protein expression using HASMCs as a positive control was determined through immunofluorescence detection of αSMA and MYLK antibodies (FIGS. 7A, 7B). Morphologically, ASCs cultured in presence of general growth media appear flat and elongated with multiple extensions resulting in a total cell area of 4550±2160 μm². While AngII cultured ASCs did not differ significantly in shape and size (5250±2390 μm²), ASC treated in AngII+SMC-GM and HASMCs were significantly smaller (p<0.05) with cell areas of 1440±900 μm² and 1200±571 μm², respectively. Average cell intensity of αSMA expression was increased significantly in ASC-SMC II cells compared to undifferentiated ASCs and ASC-SMC I cells indicating that the addition of the cytokines in the SMC GM significantly increased SMC differentiation over AngII alone. However, αSMA expression was significantly higher in HASMC controls compared to ASC-SMC II cells (p<0.05). MYLK protein expression in ASC-SMC I cells was significantly higher relative to undifferentiated ASCs (p<0.05), producing intensities comparable to HASMCs. Additional expansion in SMC GM further increased MYLK production (p<0.05). Due to the favorable results of the ASC-SMC II group, this SMC differentiation protocol was used from here on.

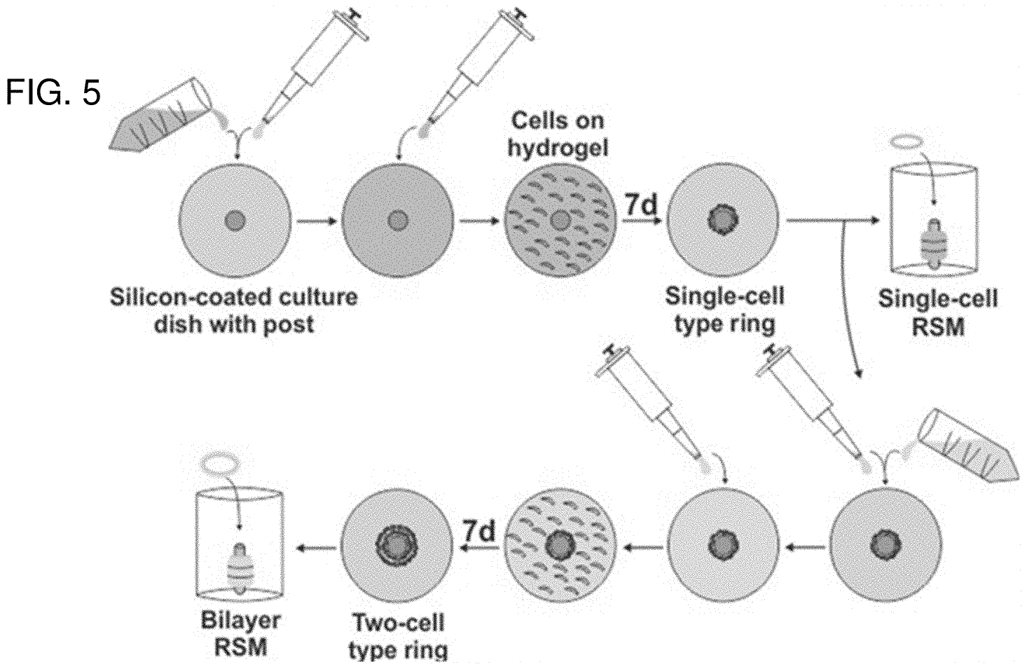

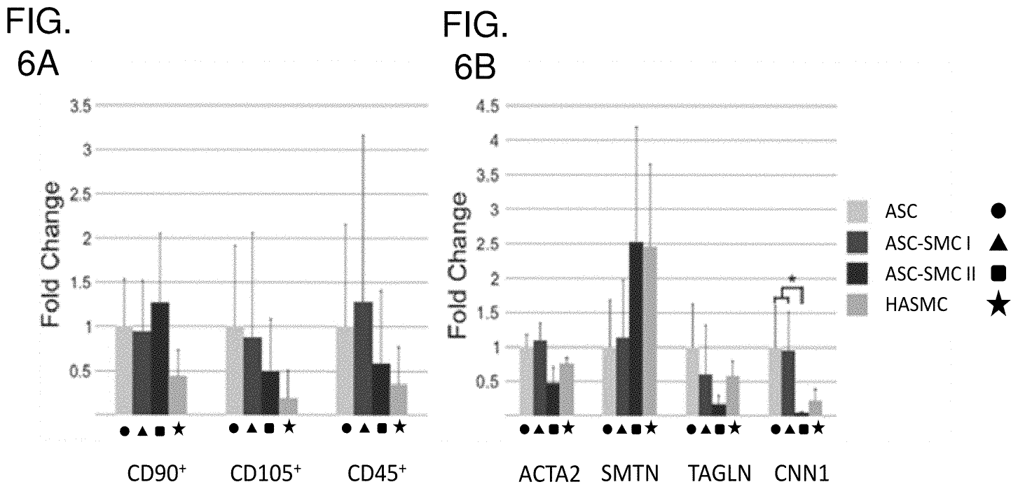

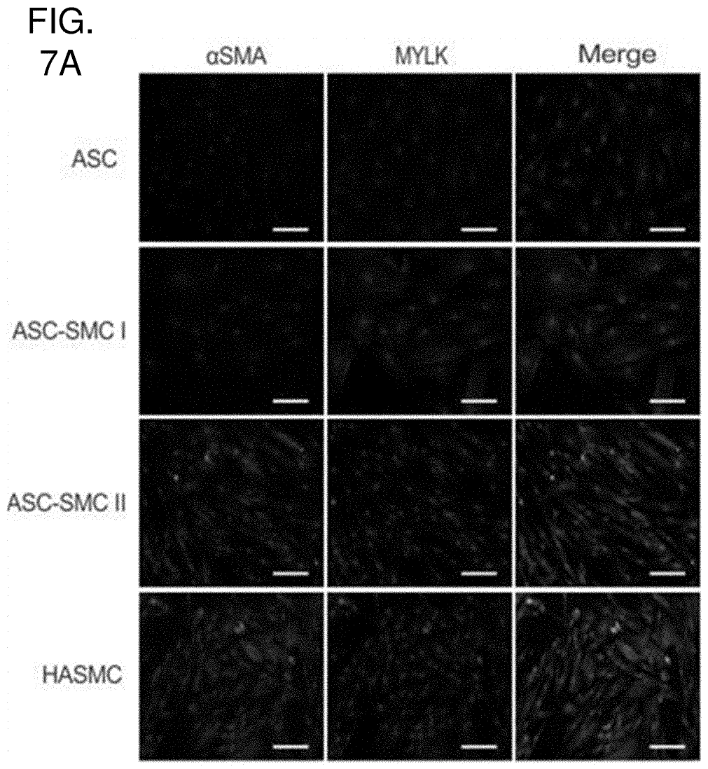

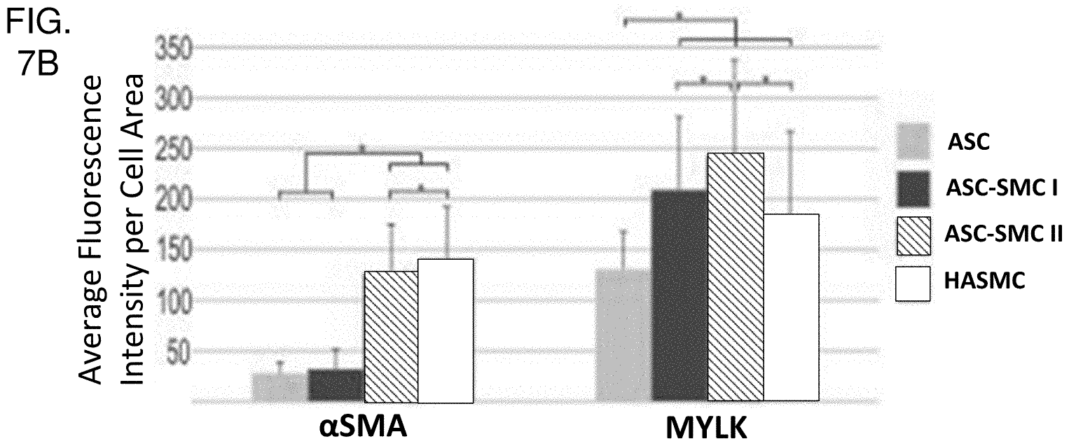

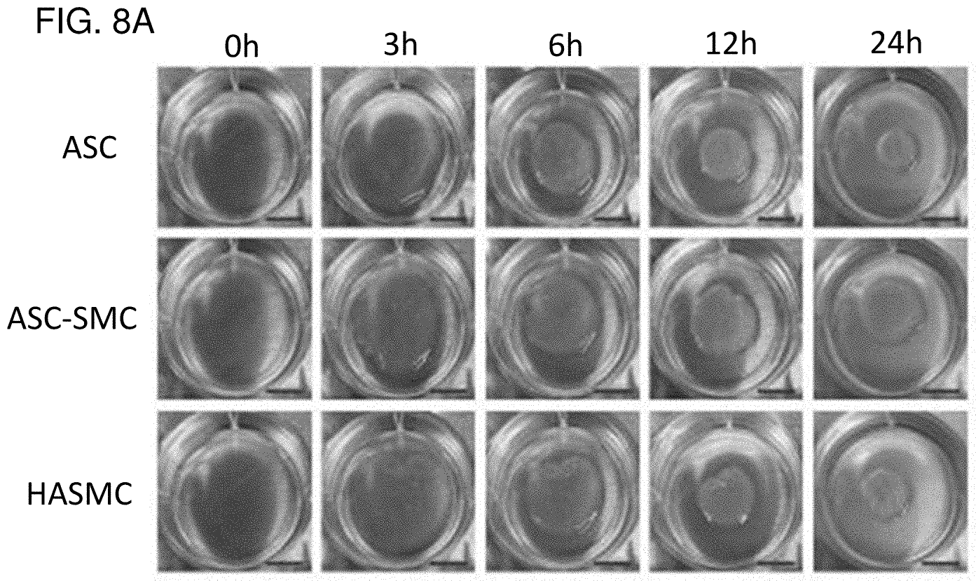

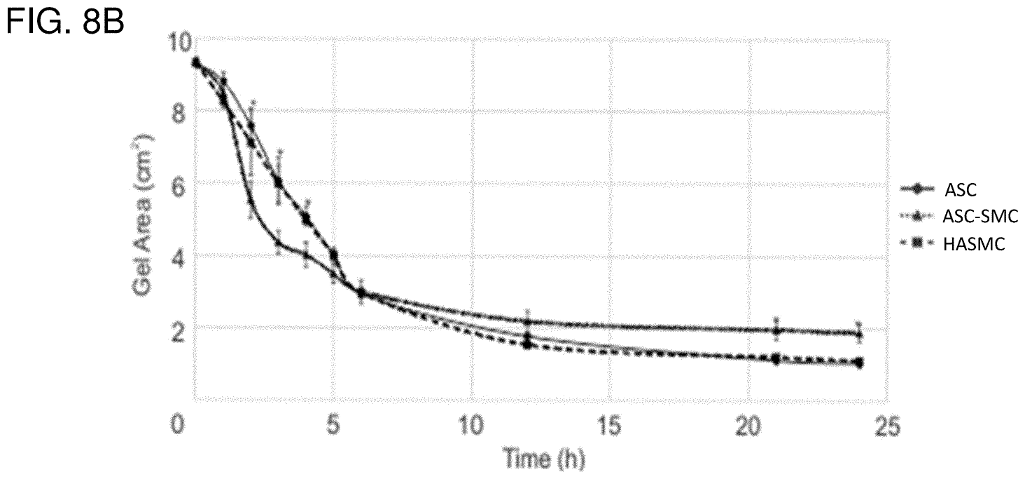

Functionality of contractile proteins was assessed through cellular contraction of fibrin hydrogels. Representative images of cellularized hydrogels during sequential timepoints were collected (FIGS. 8A, 8B). Initially, ASC-SMC II cells contracted gels significantly more than undifferentiated ASCs and HASMCs at 2-4 h time points (p<0.05). At 6 h, all gels had similar areas. At 21 h and 24 h time points, ASC-SMC II seeded gels were significantly larger (p<0.05) than ASC and HASMC samples.

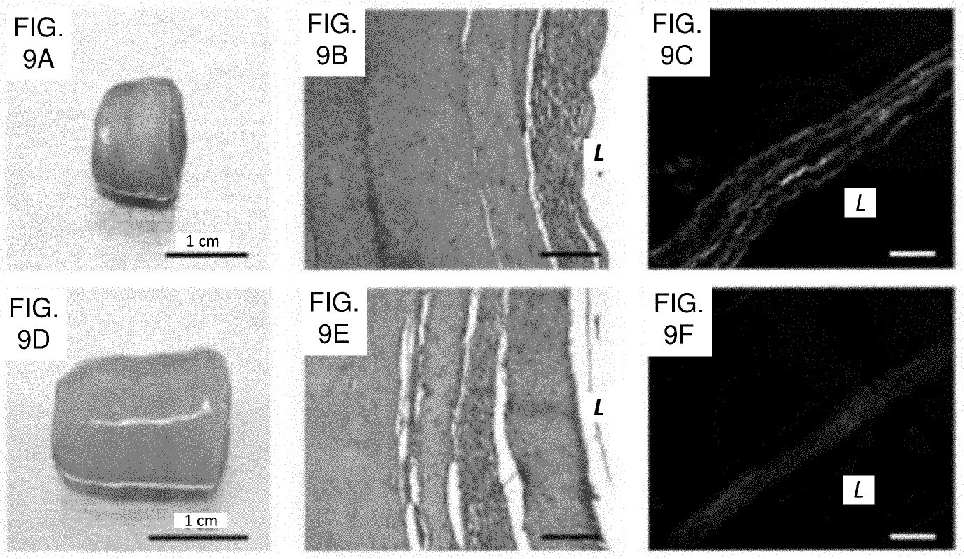

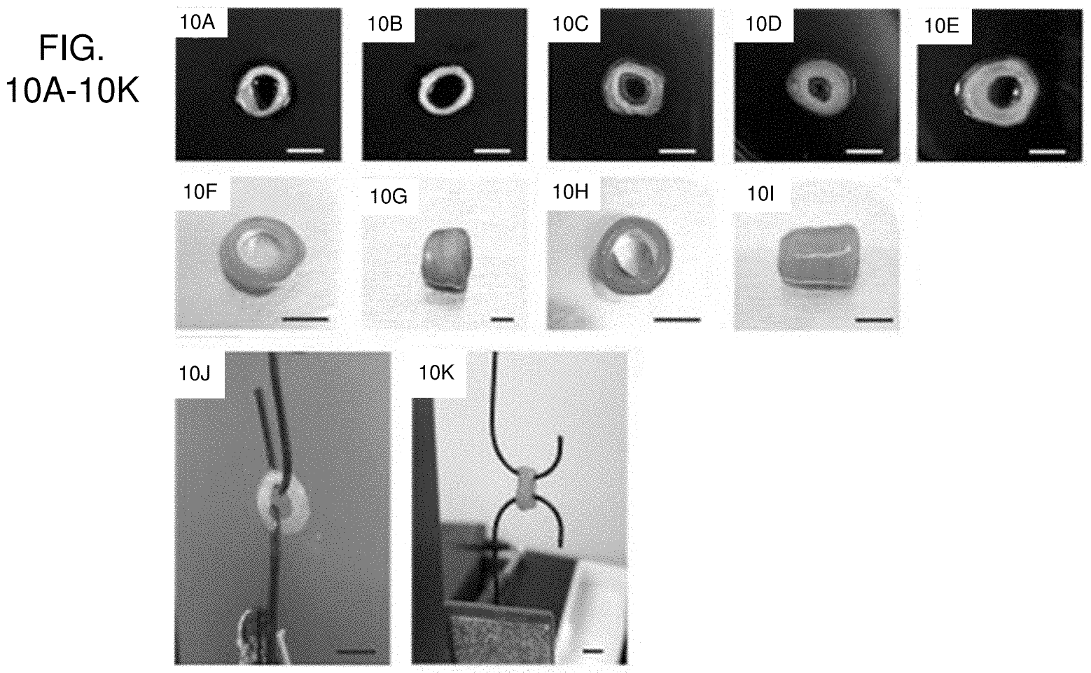

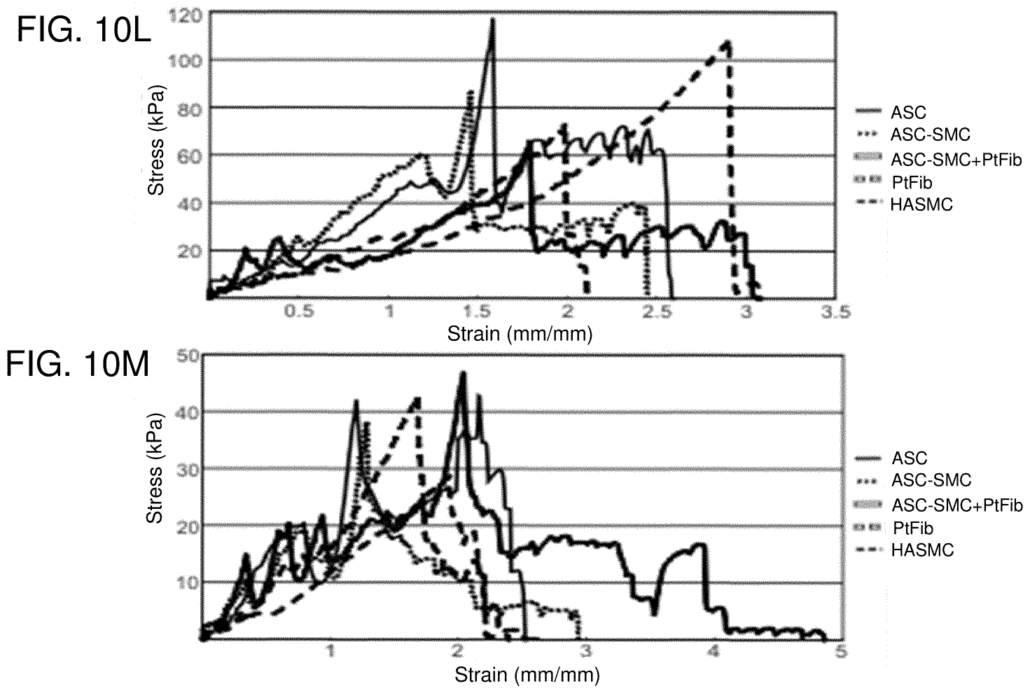

ASC-SMCs Increase Elasticity of Engineered Vascular Tissues. Circumferential tensile mechanics of vascular rings varied significantly between cell types as shown by average stress-strain curves (FIGS. 10A-10F) and mechanical properties summarized in Table 2. ASC-SMC II differentiation resulted in a significantly higher elasticity at an elastic modulus of 75.0±37.4 kPa compared to undifferentiated ASCs at 118±75.9 kPa, indicating decreased stiffness following differentiation into the vascular smooth muscle phenotype. Both ASC-SMC II and undifferentiated ASC rings exhibited significantly higher elastic modulus than HASMC's elastic modulus of 28.3±8.69 kPa (p<0.05), indicating that HASMC tissues exhibited the highest elasticity. However, the ultimate tensile strength of ASCs of 109±51.4 kPa and ASC-SMCs of 84.6±34.4 kPa rings did not differ significantly, and were comparable to that of HASMC rings of 105±26.8 kPa.

TABLE 2
Average circumferential tensile properties of patient cell vascular tissue rings.

| Ring Cell | | | | | |
|---|---|---|---|---|---|
| Type | E (kPa) | UTS (kPa) | FS (kPa) | Max F (N) | Elongation (%) |
| ASC | 118 ± 75.9 ^{b, c, d, e} | 109 ± 51.4^{d, f} | 71.8 ± 32.7 ^{c, e} | 0.186 ± 0.068 | 212 ± 51.0 ^{c, e} |
| ASC-SMC | 75.0 ± 37.4 ^{a, c, d} | 84.6 ± 34.4 ^{d, f} | 57.0 ± 26.5 ^{c} | 0.133 ± 0.069^{c} | 213 ± 49.3 ^{c, e} |
| HASMC | 28.3 ± 8.69 ^{a, b, e} | 105 ± 26.8^{d, f} | 104 ± 26.3 ^{a, b, d, e} | 0.230 ± 0.063 ^{b, d, e} | 272 ± 29.6 ^{a, b, d} |
| PtFib | 33.2 ± 14.5 ^{a, b, e} | 71.5 ± 26.8 ^{a, b, c} | 66.6 ± 36.7 ^{c, e} | 0.134 ± 0.058^{c} | 219 ± 51.2 ^{c, e} |
| Bilayer (ASC- | 77.0 ± 46.0 ^{a, c, d} | 63.5 ± 31.4 ^{a, b, c} | 42.8 ± 17.8 ^{a, c, d} | 0.154 ± 0.065 ^{c} | 262 ± 51.4 ^{a, b, d} |
| SMC + PtFib) | | | | | |

^{a}Statistically significant difference relative to ASC (p < 0.05).
^{b}Statistically significant difference relative to ASC-SMC (p < 0.05).
^{c}Statistically significant difference relative to HASMC (p < 0.05).
^{d}Statistically significant difference relative to PtFib (p < 0.05).
^{e}Statistically significant difference relative to Bilayer (p < 0.05).

Furthermore, HASMC rings yielded a higher failure strength and percent elongation relative to ASC and ASC-SMC rings. Compared to other single layer rings, PtFibs had a significantly lower average ultimate tensile strength of 71.5±26.8 kPa and an elastic modulus of 33.2±14.5 kPa. Interestingly, the combination of ASC-SMC and PtFib layers to generate bilayer rings significantly reduced ultimate tensile strength relative to ASC, ASC-SMC and HASMC tissues. Important to note is that this effect was not due to a decrease in force withstood by the engineered rings but rather to the increase in cross-sectional area of the bilayer rings. The average maximum force of bilayer rings of 0.154±0.0650 N did not differ significantly from ASC, ASC-SMC, or PtFib single layer rings, which averaged 0.186±0.0680 N, 0.133±0.0690 N, and 0.134±0.0580 N, respectively. Moreover, bilayer rings averaged an elongation of 262±51.4% before failure which was significantly higher than ASCs, ASC-SMCs, and PtFibs.

Vessel mechanics were assessed with 3-ring vessels. Mechanical analysis was performed 2 days following ring stacking (FIGS. 10A-10F; Table 3). Compared to individual rings, the ultimate tensile strength, failure strength, and elastic modulus of all vessels were comparatively lower due to a four-fold increase in cross-sectional area while average maximum force increased by two-fold. Additionally, due to individual rings rupturing at different stresses, two failure points, initial tearing at failure strength 1 (FS1) and complete breakage at failure strength 2 (FS2), were recorded as both provide information important to clinical applications. Surprisingly, ultimate tensile strength, maximum force, and both failure strengths did not differ statistically between the groups, suggesting similarity in mechanical properties of the HASMC and ASC-derived engineered vessels. The average ultimate tensile, elastic modulus, primary and secondary failure strength of undifferentiated ASC vessels were 41.3±15.7 kPa, 71.3±35.3 kPa, 38.7±18.1 kPa, and 18.2±6.22 kPa, respectively. ASC-SMC vessels had average ultimate tensile strength, elastic modulus, primary and secondary failure strengths of 37.3±17.0 kPa, 45.2±18.9 kPa, 34.5±19.5 kPa, and 6.02±1.91 kPa, respectively. Elastic moduli of vessels composed of PtFibs, HASMCs, and bilayers consisting of ASC-SMCs and PtFibs were significantly lower relative to ASCs with average values of 27.7±7.49 kPa, 18.7±5.49 kPa, and 35.2±25.3 kPa (p<0.05). Furthermore, bilayer vessels had significantly longer elongation before secondary failure, relative to ASC, HASMC, and PtFib vessels, with an average of 311±57.0%. Overall, culturing ASCs in differentiation medias prior to ring and vessel fabrication primarily effected elastic modulus of tissue cultures while maintaining average ultimate tensile strength. Formation of a tunica media-adventitia bilayer did not significantly improve ultimate tensile strength, however, bilayer rings and vessels improved elongation before failure relative to PtFib groups and ASC-SMC rings.

TABLE 3
Average circumferential tensile properties of patient cell vascular tissue vessels.

| Vessel | | | | | | Elongation |
|---|---|---|---|---|---|---|
| Cell Type | E (kPa) | UTS (kPa) | FS1 (kPa) | FS2 (kPa) | Max F (N) | (%) |
| ASC | 71.8 ± 35.3 ^{b, c, d} | 41.3 ± 15.7 | 38.7 ± 18.1 | 18.2 ± 6.22 | 0.359 ± 0.165 | 212 ± 35.3 |
| ASC-SMC | 45.2 ± 18.9 | 37.3 ± 17.0 | 34.5 ± 19.5 | 6.02 ± 1.91 | 0.341 ± 0.117 | 285 ± 55.0 |
| HASMC | 18.7 ± 5.49^{a} | 28.4 ± 11.2 | 28.2 ± 11.4 | 15.1 ± 12.7 | 0.295 ± 0.079 | 230 ± 34.3 |
| PtFib | 27.7 ± 7.49^{a} | 41.7 ± 26.1 | 40.7 ± 26.4 | 11.8 ± 6.32 | 0.267 ± 0.175 | 210 ± 49.2 |
| Bilayer | 35.2 ± 25.3^{a} | 45.7 ± 13.3 | 45.6 ± 13.3 | 10.1 ± 7.45 | 0.272 ± 0.103 | 311 ± 57.0 ^{a, b, c} |
| (ASC-SMC + | | | | | | |
| PtFib) | | | | | | |

^{a}Statistically significant difference relative to ASC (p < 0.05).
^{b} Statistically significant difference relative to HASMC (p < 0.05).
^{c} Statistically significant difference relative to PtFib (p < 0.05).
^{d} Statistically significant difference relative to Bilayer (p < 0.05).

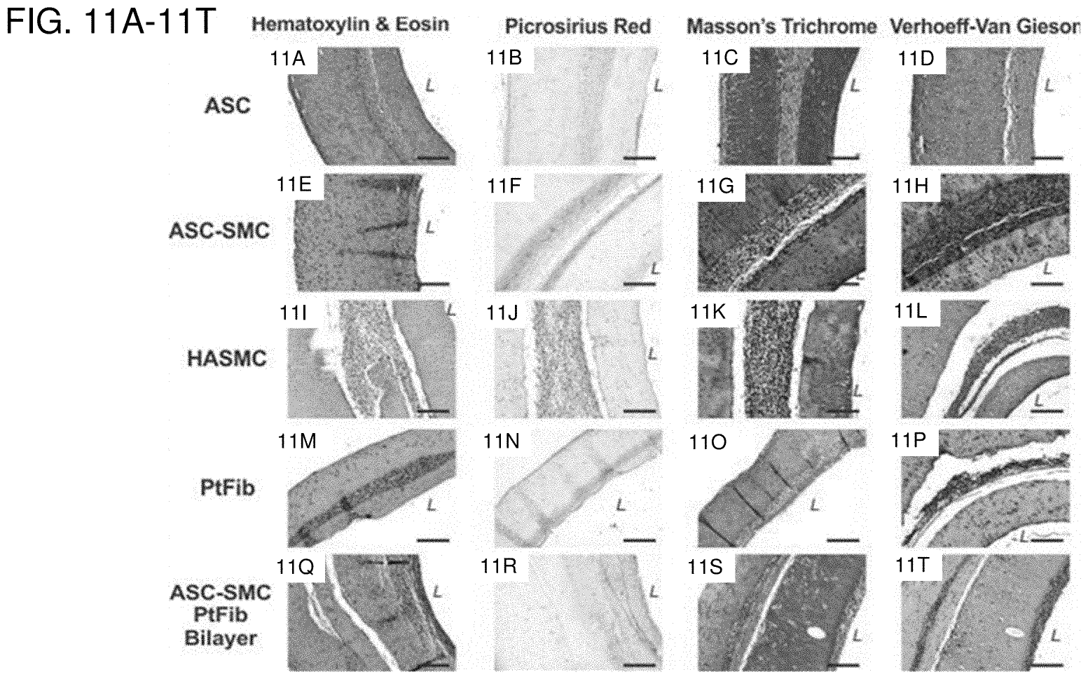

ASCs Significantly Increase Collagen Content in Engineered Vascular Tissues. Cellular organization, extracellular matrix content, and contractile protein expression were identified through staining of tissue engineered rings (FIGS. 11A-11T). After 7 days in culture, cell monolayers in all cell groups aggregated into self-assembled rings with a provisional fibrin gel coating to aid in stabilizing the monolayer as it aggregated into rings. The cells in the rings aligned circumferentially around the central post in the plate. Ring thickness varied slightly.

Collagen was noticeably abundant in the ASC and ASC-SMC rings, while HASMC and PtFib rings appeared to have less collagen deposition. Image analysis quantification of the percentage of collagen content per total ring area confirmed these observations. Collagen content in HASMC, PtFib, and ASC-SMC bilayer rings made up 12.6±4.79%, 13.7±3.47%, and 16.4±4.69% of total ring area, which was significantly less than both ASC based groups (p<0.05). ASC-SMC rings contained significantly more collagen per ring area with an average of 39.9±10.4% relative to the 29.2±4.54% observed in ASC rings. Under polarized light, collagen fibers in ASC rings displayed green whereas collagen in ASC-SMC rings appeared primarily green and yellow signifying more maturity. Collagen fibers were nearly undetectable in PtFib and HASMC samples. In ASC-SMC bilayer vessels, more mature collagen fibers, indicated by yellow and red, were present in the ASC-SMC ring. The differences seen in the bilayer ring versus ASC-SMC may have been from the extended culture of the ASC-SMC ring used in bilayer fabrication allowing more time for cells to deposit and remodel collagen. Expectedly, Verhoeff-Van Gieson stains revealed absence of elastic fibrils within all groups (FIGS. 11A-11T). Elastogenesis in cell culture has been shown to require prolonged periods under dynamic, stretch-inducing culture regimes (Isenberg & Tranquillo, Ann Biomed Eng 31, 937-949, 2003). Elastogenesis in culture was previously explored in our lab with the conclusion that growth factor techniques currently available are insufficient to provide feasible elastin amounts for vascular application.

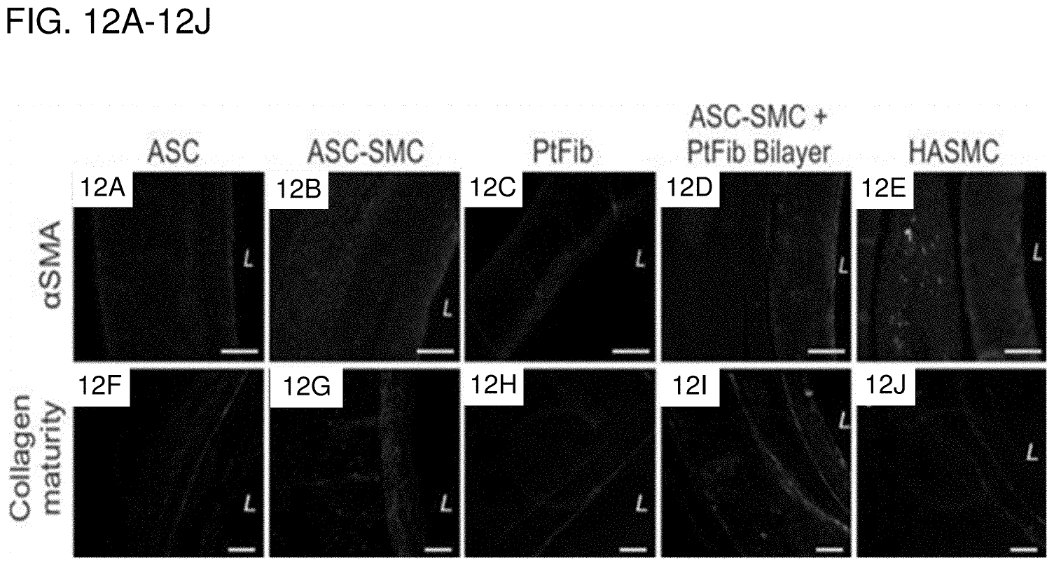

The effects of tissue fabrication on cellular production of αSMA was determined through immunofluorescence imaging (FIGS. 12A-12J). PtFib and HASMC rings were used as negative and positive controls, respectively. Despite limited production seen in 2D cultures, ASC rings stained positive though with limited expression. The limited stain present in the primary cellular layer of ASC and PtFib rings was expected as methods for ring formation requires minute concentrations of TGF-β1, a known stimulator of αSMA (Hu et al., Am J Respir Cell Mol Biol 29, 397-404, 2003). ASC-SMC tissues were also stained positive for αSMA. Notably, a distinct difference in αSMA intensity can be seen between ASC-SMC and PtFib layers within the bilayer ring.

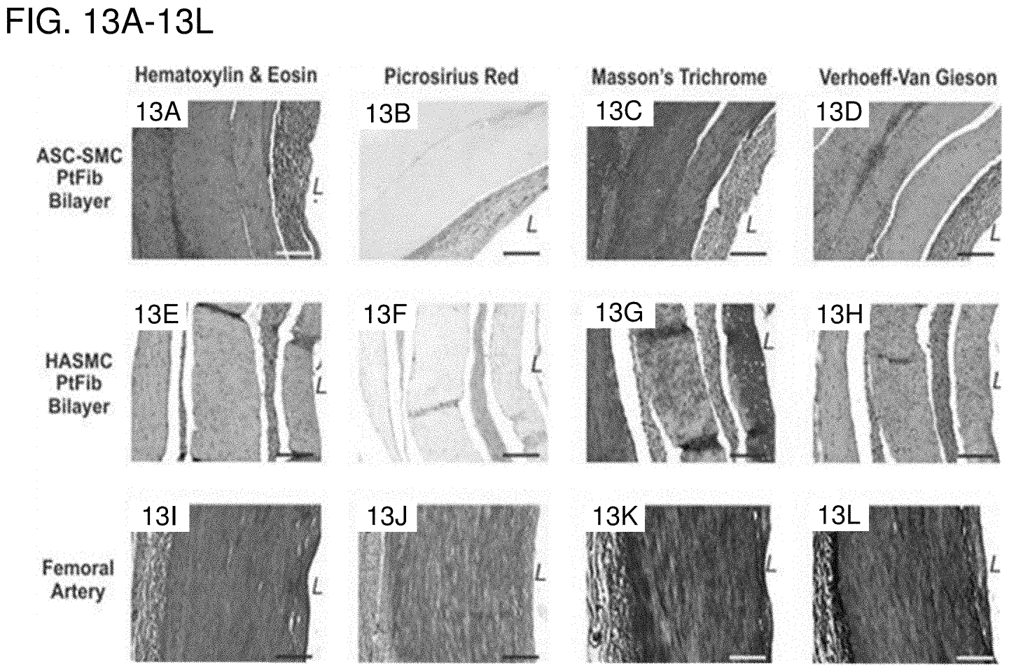

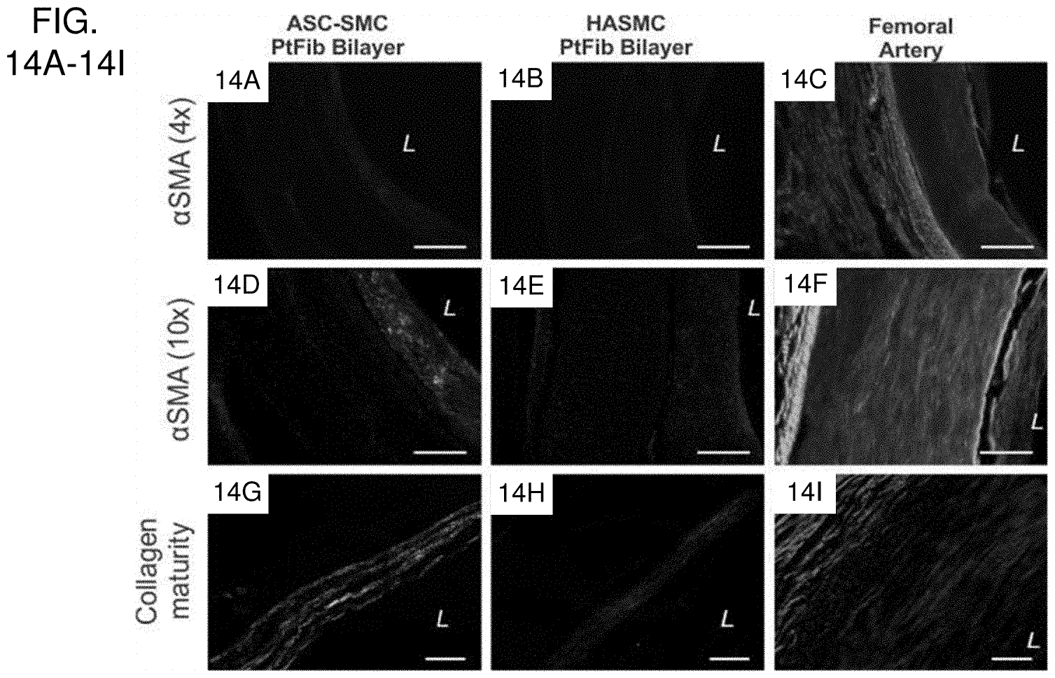

Long-Term Culture Bilayer Vessel Histological Analysis. Vessel long-term viability and matrix remodeling were assessed in bilayer vessels comprised of a PtFib tunica adventitia and either ASC-SMC or HASMC derived tunica media (FIGS. 13A-13L). Histologically, engineered tissues preserved circumferential alignment in both distinct layers as shown by H&E stains. As desired, ASC-SMC bilayers degraded the provisional luminal side fibrin gel and the cellular layers proliferated, creating a more robust tubular structure indicative of a native vessel. Sections of a fresh cadaver femoral artery were used as the reference for native artery organization and extracellular matrix content. Prolonged culture resulted in significant deposition of collagen isolated to the cellular areas, as shown by the red and blue stained fibers. Quantification of the picrosirius stained tissues determined the collagen content per area in ASC-SMC bilayers was an average of 23.1±3.98%, which was significantly higher than the 14.5±2.26% present in HASMC bilayers (p<0.05). Additionally, polarized light showed the collagen fibers present in ASC-SMC bilayers were more mature compared to those in HASMC bilayers (FIGS. 14A-14C). However, fresh un-preserved cadaver femoral artery samples exposed to polarized light indicated that the media layer of native arteries contains primarily immature green fibers while the adventitia contains mature orange-red collagen. Elastin deposition was not present in either engineered bilayer while large elastin fibrils are seen in the native femoral artery. This finding was expected, as it is difficult to induce cellular production of elastin in cell culture.

Retainment of contractile smooth muscle phenotype following long-term static culture was determined through immunofluorescence detection of αSMA in ASC-SMC and HASMC using the femoral artery as a positive control (FIGS. 14D-14I). Both bilayer groups demonstrated positive stains near the lumen within the tunica media and less intensity with the PtFib derived tunica adventitia. Interestingly, αSMA intensity was more prominent in the ASC-SMC bilayer relative to the HASMC bilayer.

### DISCUSSION

This work demonstrates the feasibility of utilizing patient dermal fibroblasts and adipose-derived stem cells for creating a patient-specific tissue engineered vascular graft. The key factors addressed here were determining a harvestable, abundant source of autologous cells; identifying a viable vascular differentiation protocol to produce vascular smooth muscle cells; and establishing a vascular tissue engineering method conducive to patient cell application.

Viable autologous cell sources have been explored for decades. Many studies have investigated the utility of various stem cell sources, though the field has yet to solidify a workable solution. We identified tissues easily harvestable by investigating tissues commonly extracted during cosmetic surgeries, namely skin and adipose tissue. Dermal fibroblasts are easily harvested from a small skin sample from a patient. Adipose tissue is commonly extracted from patients through liposuction procedures. In this work, patient dermal fibroblasts and ASCs were matched to the same patient for each vessel, thus creating an authentic patient-specific vessel.

The key to determining the usefulness of these cells lays in their feasibility for vascular application. ASCs were explored previously in for their use in the adventitia, however, in these studies the fibroblast served better at this role as the primary cell type in the adventitia, hence dermal fibroblasts were deemed more relevant and closer to the desired phenotype. Fibroblasts from different anatomical tissues do vary somewhat in phenotype, however, their primary function of collagen production is mostly conserved allowing for fibroblasts from other tissues to be applied to the collagen-rich tunica adventitia. Advantageously, ASCs are easily harvestable adult stem cells. The next step was then to characterize their differentiation potential into vascular smooth muscle cells to fabricate the engineered tunica media. The final layer, the tunica intima, is composed of endothelial cells (ECs). There is currently no source of harvestable adult autologous ECs. In research studies, human umbilical vein endothelial cells (HUVECs) are the standard, however these cells have a vastly different phenotype from vascular ECs. Fortunately, the human body naturally endothelializes any tubular-shaped structures placed into the circulatory system (Kirkton et al., Sci Transl Med 11, 2019; Koobatian et al., Biomaterials 76, 344-358, 2016). This naturally occurring phenomenon can be employed to optimally create an intimal layer in the engineered bilayer vessels composed of a media and adventitia, hence the intima will potentially be automatically established once the graft is implanted. Therefore, the intima was not included during development of this patient-specific vascular graft.

Previous studies have shown angiotensin II facilitates the production of smooth muscle-related contractile protein in ASCs (Kim et al., Int J Biochem Cell Biol 40:2482-2491, 2008). In the present work, ASCs cultured in AngII for 7 d did not significantly alter αSMA gene or protein expression, however, myosin light chain kinase (MYLK) expression was increased as determined by immunofluorescence antibody detection (FIGS. 7A, 7B). MYLK is an enzyme responsible for phosphorylation of myosin II during muscle contraction thus represents functional capacity. An adaptation to this differentiation protocol was established to promote cell proliferation following AngII stimulation by culturing cells in smooth muscle growth media following AngII differentiation. This process drastically increased αSMA and MYLK antibody signal intensity and prompted cell morphology progression towards the fusiform shape present in HASMCs. Interestingly, despite the increase in protein expression detected through immunofluorescence, gene expression of all smooth muscle related proteins except smoothelin was found to be higher in undifferentiated ASCs relative to AngII+SMC-GM treated ASCs and HASMCs controls (FIGS. 7A, 7B). This finding may be due to ASCs deriving from the same mesenchymal germ layer as muscle cells, hence conservation of some genes is observed. Additionally, functional contractility in undifferentiated ASCs was nearly identical to HASMCs in fibrin gel contraction assays, while AngII+SMC-GM treated ASCs initially contracted gels significantly more followed by limited change after 12 h (FIG. 8A, 8B). Overall, these results correlate with others studying ASC differentiation towards the smooth muscle lineage in that contractile protein expression can be increased through biochemical factors, however, variability between donor tissues may need to be considered (Lau et al., Tissue Eng Part A 25:936-948, 2019).

The use of undifferentiated ASCs and pre-differentiated ASCs treated with the AngII+SMC-GM protocol (ASC-SMC IIs) as a smooth muscle source for tissue engineering was further assessed by tissue mechanical and histological properties. The primary difference between both ASC-based modular tissue rings and HASMC-derived rings was the elastic modulus (FIGS. 10A-10F; Table 2). ASC-SMC rings had significantly lower average elastic modulus than ASC tissues, though HASMCs were significantly lower than both groups. Histological analyses showed the increased rigidity of ASC-based rings was primarily due to the significant deposition of collagen produced by both ASC groups (FIGS. 11A-11T). ASC-SMC II differentiation resulted in a significantly lower elastic modulus of 75.0±37.4 kPa compared to undifferentiated ASCs to 118±75.9 kPa, meaning that ASCs differentiated into SMCs exhibited higher elasticity, similar to smooth muscle phenotype. Differentiated ASC rings exhibited a much higher elastic modulus compared to HASMC rings, denoting that the differentiated ASCs' phenotype lays mechanically in between undifferentiated ASCs and HASMCs. This “in-between” elastic phenotype may be advantageous in a vascular milieu where tissue compliance is key to proper vascular function. The αSMA expression of differentiated ASCs was in between undifferentiated ASCs and HASMCs, further indicating their phenotype. Fortunately, the ultimate tensile strength, maximum force, and both failure strengths did not differ statistically between the groups, indicating that the strength exhibited by tissues composed of differentiated ASCs was similar to that of HASMCs.

In addition to assessing the use of patient cells, this work adapted this lab's previous modular tissue engineering techniques to create tunica media-adventitia bilayers. Bilayers were successfully constructed. ASC-SMC and HASMC rings and vessels showed positive immunofluorescence staining for αSMA. Unexpectedly, the addition of a fibroblast adventitia exterior ring significantly reduced the ultimate tensile strength of the ASC-SMC+PtFib bilayer rings relative to ASC-SMC rings, although this effect was not present in vessels. This may be explained by the significant difference in average cross-sectional area between ASC-SMC (1.61±0.472 mm²) and bilayer tissue rings (2.48±0.471 mm²), which was negligible in vessels, since the average maximum force between both groups in ring and vessel form were not significantly different. The major contribution of strength in bilayer tissues was attributed to the ASC-SMC layer as PtFib rings and vessels had the lowest average maximum force and exhibited limited collagen production. Fibrin glue that coated the vessels provided a provisionary scaffold that is gradually remodeled and replaced by extracellular matrix deposited by the cells over time, thus creating one complete, biological tubular structure.

One month culture of bilayer vessels composed of a PtFib-derived tunica adventitia and either ASC-SMC or HASMC tunica media showed significant deposition of collagen within the cellular regions (FIGS. 13A-13L, FIGS. 14A-14I). Compared to ASC-SMC bilayer rings cultured for 1 d, longer term culture ASC-SMC bilayers preferentially degraded the luminal side fibrin gel and cellular layers proliferated, increasing the cellular content. The engineered media and adventitia remained separate after long-term culture as two distinct cellular regions as seen in all samples. Additionally, elastin deposition was not present in any tissues in this study. In vitro elastogenesis has been challenging throughout the field of tissue engineering. Studies have shown elastin production requires prolonged culture under mechanical stresses such as pulsatile perfusion or cyclic stretch (Isenberg & Tranquillo, Ann Biomed Eng 31, 937-949, 2003).

In terms of clinical application, adipose tissue is feasibly harvested from patients using liposuction procedures. Lipoaspirated adipose tissue has viably produced ASCs (Borrelli et al., Stem Cells Transl Med 9, 1389-1400, 2020; Deleon et al., J Tissue Eng Regen Med 15, 1105-1117, 2021). Dermal fibroblasts are regularly harvested in the clinic with skin samples (Elseth & Nunez Lopez, Wound Grafts. In: StatPearls. StatPearls Publishing, 2022). Hence, autologous cell harvest is possible for the cell types tested in this work, thus demonstrating the efficacy of producing patient-specific vascular grafts.

### Conclusion

This Example demonstrated the efficacy of ASCs and dermal fibroblasts as viable autologous cell sources for generating patient-specific vascular grafts. ASCs were successfully differentiated into vascular smooth muscle cells to engineer the tunica media. Patient fibroblasts were applicable to engineering the tunica adventitia. Advantageously, ASCs significantly increased collagen content and maturity in the engineered vessels. Combined, the engineered media and adventitia formed a complete patient-specific vessel characterized by increased elasticity for compliance needed for blood flow.

### Example 3: Patient-Specific Biological Engineered Blood Vessels Based on Adipose Stem Cells and Fibroblasts

Tissue engineered vascular grafts aim to solve the mechanical and immunological shortcomings of traditional synthetic grafting materials. Current tissue engineering methods often focus on recreating a single vessel layer and rely on decellularization techniques to remove allogeneic cells prior to implantation. A vascular graft replicating the multilayered architecture of vasculature using biologic materials and cells derived from the patient would solve the major issues. However, a lack of a viable patient cell source for smooth muscle cells and fibroblasts has hindered the development of cellularized vascular grafts. Here, the Ring Stacking Method is adapted to establish tunica media-adventitia biological engineered blood vessels sourced from minimally-invasive adipose tissue.

Materials and Methods: Abdominoplasty tissues were utilized to procure adipose mesenchymal stem cells (ASCs) and dermal fibroblasts for recreating the tunica media and adventitia, respectively. ASCs were stimulated with angiotensin II and smooth muscle growth factors towards a contractile smooth muscle-like phenotype (ASC-SMCs). Engineered vascular tissue was first constructed into rings using ASC-SMCs or patient fibroblasts seeded into a custom plate with a central post around which the cell monolayer would form the ring structure. Bilayer tissues were established by repeating this process with a pre-formed tunica media ring embedded in a tunica adventitia hydrogel. Following ring formation, tissues were assembled on a 3D-printed polylactic acid post and adhered together with fibrin to form vessels. The mechanical and histological properties of vascular tissues made from each cell type, as well as aortic smooth muscle cells (SMCs), were assessed. Additionally, bilayer vessels composed of ASCs and fibroblasts or SMCs and fibroblasts were analyzed histologically following long-term culture.

Results and Discussion: Stimulation of ASCs by angiotensin II and smooth muscle growth factors resulted in greater expression of smooth muscle contractile proteins myosin light chain kinase and α-smooth muscle actin as determined by immunofluorescence. ASC-based tissues exhibited increased collagen deposition and fiber maturity compared to SMCs. Mechanically, ASC-based tissues had similar ultimate tensile strength and higher elastic moduli than SMC controls. Bilayer vessels displayed two distinct cellular layers though ASC-based bilayers showed drastic collagen maturation relative to SMCs (FIGS. 9A-9F).

Conclusion: This example successfully established methods for creating bilayer vascular tissues via the Ring Stacking Method. Additionally, the mechanics of ASC-based tissues show their potential as a vascular smooth muscle cell substitute for tissue engineering.

### Example 4: Induction of Ealy Stage Atherosclerosis in a Small Two Cell Layer Vessel with Late Stage Calcification Computational Validation

Atherosclerosis is characterized by plaques which occlude the vessel and severely block blood flow as the disease progresses (Saez et al., Comput Mech 53: 1183-1196, 2014). Plaques are caused by elevated low-density lipoprotein (LDL) in the blood, which initiates an immune response cascade leading to severe arterial stiffening due to calcification. Calcification is the main factor affecting vessel mechanics in atherosclerosis. During disease progression, the smooth muscle cells located in the tunica media release calcium into the surrounding tissue, calcifying the plaques. Over time, the calcified plaques increase in size leading to occlusion of the artery. There are two types of calcified plaques, defined by their shape: concentric and eccentric (German & Madihally, Computation 4, 7:1-11. 2016). Concentric plaque formation is a symmetric calcification and loss of compliancy along the arterial wall. Eccentric plaque formation is an asymmetric calcification and loss of compliancy along the arterial wall, where one side may have little to no stiffening at all. To better understand how to construct a 3D tissue engineering disease model using a BEBV singular rings created via the RSM, we must learn how create these pathological changes in vitro.

The first step towards creating an atherosclerosis disease model, one must mimic the inflammation response associated with the illness. One study used a co-culture of endothelial cells (ECs) and smooth muscle cells (SMCs) in along with monocytes to achieve foam cell development (Takaku et al., Arteriosclerosis and Thrombosis 19:2330-2339, 1999). The researchers plated a layer of collagen, followed by a final seeding of ECs on top. They mixed ox-LDL into the collagen layer between the ECs, for it to act as the sub-endothelial space. They observed monocyte transmigration through the EC monolayer along with macrophage differentiation and foam cell formation. A similar study discovered that adding a EC+collagen+SMC model elicited increased monocyte transmigration (Navab et al., J. Clin. Invest. 82: 1953-1863, 1988). This was compared to a collagen and EC+collagen model that relies on a chemoattractant for monocyte transmigration. Via transverse sections they witnessed numerous monocytes in the sub-endothelial space after adhesion to the EC monolayer. These two groups were able induce monocyte transmigration using a Cartesian co-culture models but failed to create a fibrous cap that is necessary for the occlusion of the vessel.

Computational simulations are a useful tool to validate experimental procedures and results for benchtop models of arterial disease (He et al., 2017). Computed tomography (CT) images of existing artery networks explanted from a cadaver can be used to create a 3-dimensional computer model (Kaski et al., JACC 17(3):627-33, 1991). Hemodynamics of an artery can be modeled using a finite element analysis, under the assumptions that the system is laminar, Newtonian, viscous, and has incompressible blood flow with a linear-elastic, isotropic, and incompressible vessel wall (Boutsianis et al., E J Cardio-thoracic Surgery 26: 248-256, 2004). Using the Navier-Stokes equations, the fluid dynamics can be calculated for flow and pressure through the arteries in the presence of the concentric and eccentric occlusion models (Sun & Xu, Computerized Medical Imaging and Graphics 38: 651-663, 2014). Due to the hyper-elastic nature of the collagen fibers in the extracellular matrix of the arterial wall, the tissue will take on a non-linear behavior which can be mathematically modeled using the Money-Rivlin equation (Karimi et al., Bioengineered 8(2):154-170, 2017).

The aim of this Example was to create a tissue engineered atherosclerotic calcification model correlated to a computer simulation and to begin early immune response model using smooth muscle cells, endothelial cells and monocytes in vitro. The main purpose of the computer simulation was to augment the data provided by the bench-top engineered model to provide information beyond the capabilities of the engineered model. Specifically, the engineered model was used to test application of calcification techniques to an actual tissue model. Next, the computer simulation was used to validate the experimental calcification techniques, and then to model concentric and eccentric plaque formation. The computer simulation was able to be easily extended modeling capabilities into an area challenging for the bench-top model to duplicate. The advantage our laboratory has is that we are able to readily correlate computer modelling with benchtop data obtained from our lab's completely biological engineered blood vessels (BEBVs) via the RSM seen in chapter 2 and 3. Thus, we are able to collect the appropriate experimental data to input into the simulation, greatly increasing the accuracy of our model compared to common purely computational approaches.

To induce calcification into the BEBVs, calci-protein particles (CPPs) were explored. CPPs were identified as a promising candidate to induce calcification due to their ability to stimulate calcium release by smooth muscle cells (SMCs) in vitro (Aghagolzadeh et al., Atherosclerosis 251 404-414, 2016, Kelynack & Holt, Meth Mol Biol 1397: 209-220, 2016). Additionally, high CPP levels can be found in patients suffering from CAD (Nakazato et al., J Cardiology 74 428-435, 2016). CPPs are also present clinically in chronic kidney disease (CKD) patients, which correlates with a high incidence of atherosclerosis (Zeper & de Baaij, Renal Pathophysiology 28(4):368-374, 2019). The CPPs were able to be successfully integrated into the BEBVs. CPP-integrated BEBVs were uniaxially tensile tested to determine resultant stiffness.

The conditions found in the experimental BEBV model were further iterated in a cylindrical computational model of the pathology of atherosclerosis to establish optimal experimental parameters of the calcification process. Following the generation of a CPP-containing BEBV, a 3-dimensional simulation model of the engineered vessel was generated in a non-linear, hyper-elastic laminar flow simulation using ANSYS. Next, both concentric plaque buildup with 75% occlusion and eccentric plaque buildup with 75% occlusion was created to better understand the efficacy of the CPPs to simulate an occluded state. This work shows that computer simulations can serve as a valuable tool to verify, iterate and further augment pathophysiological processes applied to benchtop models. In this specific case, a computer simulation was used to improve an engineered model of atherosclerosis being developed to, in the future, test new disease treatments.

Following the computer simulated calcification model, we will combine those tissue creating methods using CPPs to create an initial in vitro tissue engineered atherosclerotic disease model using singular rings and a BEBV with two cell layers (intima and media). THP-1 monocytes will be cultured and used because they are highly characterized for atherosclerosis studies in vitro (Graham et al., Atherosclerosis. 120(1-2):135-45, 1996, Hayden et al., J Lipid Res. 43(1):26-35, 2002). During the inflammation response that initiates atherosclerosis monocytes differentiates into M0 then into either M1 or M2 macrophages (Bobryshev et al., Biomed Res Int. 2016:9582430. doi: 10.1155/2016/9582430). M1 macrophages are proinflammatory cells that further promote the recruitment of monocytes (Yang et al., Biomark Res. 2(1):1. 2014 doi: 10.1186/2050-7771-2-1). M2 macrophages are anti-inflammatory cells that inhibit the further recruitment of the monocytes (Ley, J Immunol. 199(7):2191-2193, 2017). Even though both M1 and M2 macrophages are present in plaque formations M1 macrophages and endothelial cells responsible for recruiting more monocytes. ox-LDLs will be present in our tissues to better mimic the disease state. We will determine the mechanical properties of the engineered diseased tissue later in the chapter.

### Materials and Methods

### Cell Culture

The cells utilized were human aortic smooth muscle cells (SMCs) and purchased from American Type Culture Collection (ATCC) (PCS 100-012, ATCC, VA). The cells were maintained in smooth muscle cell growth medium consisting of 88.6% Dulbeccos Modified Eagle Medium (SH30022.01, Hyclone Laboratories, UT); 0.1% each of recombinant human insulin (rH-insulin) (407709-50MG, EMD Millipore, Darmstadt, Germany), recombinant human fibroblast growth factor (rH-FGF) (100-18B, Peperotech, N.J.), recombinant human epidermal growth factor (rH-EGF) (AF-100-15, Peprotech, N.J.), ascorbic acid (A-7506, Sigma-Aldrich, MO); and 5% each of fetal bovine serum (FBS) (10437-028, Gibco, MA) and L-glutamine (BP379-100, Fisher Scientific, NJ); and 1% antibiotic/antimycotic (15240-0621, Gibco, MA). The medium was changed twice a week until the cells were ready to be seeded into engineered vascular rings as described further within. THP-1 cells were purchased from ATCC and maintained THP-1 growth medium consisting of 88.6% RPMI Medium (SH3002701, Hyclone Laboratoris, UT); 10% FBS and 1% antibiotic/antimycotic. Media for the THP-1 was changed every 3 days. HUVECs will be cultured in EC growth media composed of 91.3% 131 media, 2% FBS, 5% L-glutamine, 0.1% ascorbic acid, 0.1% recombinant human insulin-like growth factor (rH-IGF), 0.1% rH-FGF, 0.1% rH-EGF, 0.1% heparin sulfate, 0.1% recombinant human vascular endothelial growth factor (rH-VEGF), and 0.1% hydrocortisone. The media was changed every 3 days.

### Preparation of Calci-Protein Particles (CPPs)

The CPPs were produced under sterile conditions by mixing 15 ml FBS; 30 ml of 20 mM CaCl₂ (C1016-500G, Sigma-Aldrich, MO) in tris-buffered saline (TBS) (BP1757—500, Fisher Scientific, NJ); 30 ml of 14 mM NaHPO₄ (S9638-500G, Sigma-Aldrich, MO) in TBS; and 45 ml SMC growth medium in a 100 ml sterile specimen bottle. Utilizing a magnetic stir bar and plate, the solution was continually mixed slowly for 12 hours at room temperature. The solution was then aliquoted into three 50 ml tubes pipetting 40 ml of solution into each. The solution was centrifuged at 5000 rpm for 10 hours at 4° C. Following centrifugation, the supernatant was removed, and each pellet resuspended in 2 ml of SMC growth medium warmed to 37° C. creating a CPP suspension. The CPP solution can be stored at 4° C. for 2 months.

### Preparation of Plates for the Engineered Vessels

As described further herein, ring structures are created out of vascular cells then stacked to generate our engineered vessels, by the RSM. To create the ring structures, vascular cells were plated in custom-made plates with a central post around which the rings would form (herein termed the post plates). To make these plates, first, a 1:10 curing agent base polymer mixture of poly(dimethylsiloxane) (PDMS) silicone elastomer (Sylgard 184, 1064291, Dow Corning Co, MI) was prepared. Two ml of uncured silicone was added to each well of 6-well petri dish. The 6-well petri dish was placed on a level surface and allowed to initially cure for 2 hours uncovered. Next, the petri dish was placed on a hot plate set at 60° C. for 3 hours uncovered to complete curing of the silicone polymer. Posts were created by pouring PDMS into a 100 mm petri dish at a height of 7 mm and allow to cure on a hot plate at 60° C. for about 3 hours to generate a bulk piece of PDMS/

Next, a 5 mm biopsy punch (33-35, Miltex, Pa.) was used to punch out cylindrical posts to create 5 mm diameter lumen rings. A small amount of uncured PDMS was applied to the bottom of each PDMS cylinder to attach it to the center of each well. The 6-well petri dish was placed on the hot plate at 60° C. for 3 hours until cured. In a biological hood, a solution of 70% ethanol (2705, Decon Laboratories, PA) with distilled water (W5-4, Fisher Scientific, NJ) was added for 30 minutes to the inside of each well for sterilization. Then the ethanol was carefully aspirated from each well. Next, the dishes were exposed to UV light under the hood for an additional 30 minutes for further sterilization.

### Seeding Cells for Forming Vascular Rings

To form the vascular ring structures, human aortic SMCs were seeded into the post plates along with a fibrin hydrogel which served as a provisional, temporary matrix to stabilize the cell monolayer. An SMC cell suspension of 1.5×10⁶ cells/ml was created for seeding cells on top of the hydrogel. Cells at a concentration of 1.5×10⁶ cells/ml were resuspended in a solution of 20 mg/ml of fibrinogen (MP Biomedicals, CA) for adding cells inside the hydrogel. Rings were divided into two groups: the non-treated (control) and CPP (experimental) group. Hydrogel medium was defined as SMC growth medium with 0.01% TGF-β-.

To form the rings, first, 160 μl of fibrinogen with a 1.5×10⁶ cell suspension the fibrinogen was added to the 6-wells with and without CPPs (experimental and control groups, respectively), followed by 40 μl of thrombin from a stock of 100 U/ml (7592, Biovision, CA) to create the fibrin gel. The hydrogel was allowed to cure for 5-10 minutes at room temperature in the biological hood. For the CPP group, 25 μl of suspended CPP solution was added to each well and swirled for equal distribution. No CPPs were added to the wells for the control group. Next, 1.5×10⁶ cells in 1 ml of SMC growth medium was added in a circular motion to each well for both groups for seeding cells on top of the hydrogel. Lids were placed on the plates and placed in the incubator. After 12 hours, the medium was aspirated and replaced with 2 ml of fresh SMC medium. For the CPP group, 50 μl of the CPP suspension was added to each well. The medium was changed every 48 hour and fresh CPP solution added to each well during each media change for 7 days. For all groups, after 3-5 d, rings fully aggregated in towards the post forming the ring structures, following this 10 μl of TGF-β1 was added to each well for both groups.

Stacking of Rings into Engineered Blood Vessels (Ring Stacking Method)

To hold the BEBV, a specialized container called a “tall plate” was made using polycarbonate tubing, 60 mm petri dish bottom and lid. The two pieces were adhered together using a polymer solvent. A ring stacking post 5 mm in diameter and 30 mm long was 3D printed (MakerBot, Brooklyn, N.Y.) with a poly(lactic acid) (PLA) filament. Following this, 8 ml of uncured PDMS was added to the tall plate, then the 3D printed ring stacking post were placed in the center of the plate. PDMS in the tall plates was allowed to degas at room temperature for 2 h then placed onto a hot plate at 60° C. for 2-3 h to cure. A solution of 70% ethanol was added for 30 min to the inside of each tall plate to sterilize. The ethanol was aspirated, and the tall plates exposed to UV light under the hood for 30 minutes to finalize sterilization. Using very fine forceps, each engineered ring was carefully taken from its post and transferred to the stacking post of the tall plate. Each ring was gently moved circumferentially down to the bottom of the post. Six rings were stacked to constitute one engineered vessel. With the ring stack on the stacking post, the tall plate was then turned so that the post was parallel to the working surface. Using a micropipette, 40 μl of thrombin at a concentration of 100 U/ml was gently applied to the ring stack using a rotating motion. Next, 40 μl of 20 mg/ml concentration fibrinogen was added to each ring stack using a quick, rotating motion. The thrombin and fibrinogen quickly cured to form a firm gel once mixed, hence coating the entire BEBV with a thin layer of fibrin gel in the final step. Twenty ml of SMC growth medium was added to the tall plate holding the engineered vessel and placed into the incubator. Media was changed every 5-7 days until use.

### Human Tissue Controls

Human common iliac arteries (CIA) were isolated and donated from a patient with unknown age and sex who underwent an extraction their abdominal aorta-common iliac artery bifurcation due to extreme calcification and occlusion of the abdominal aorta and right CIA by way of the Vascular Surgery Clinic at Henry Ford Hospital (Detroit, Mich.). This tissue was used as control tissue for normal and calcified states in order to determine to obtain elastic moduli ratios through tensile tests.

### Creation of Diseased Tissues Rings for Atherosclerotic In Vitro Model

Diseased tissue rings were created in two methods: natural disease rings (ND) and engineered disease media rings (EDM). Natural disease rings were created in same manner as the control and CPP rings above accept 1.5×10⁶ SMCs were suspended in 340 μl of SMC growth medium instead of fibrinogen. 160 ul of Ox-LDLs was to mimic oxLDLs that have fallen in the subendothelial space. Then 40 μl of thrombin followed by 160 μl of fibrinogen to cure the hydrogel and cells. Following this 1.5×10⁶ SMCs were seeding on top with 1 ml of SMC growth medium. Rings formed after about 1 week in culture and media changes were provided similar to the above protocol and previous chapter.

EDM rings were created in the same manner as the natural disease except 1×10⁶ THP-1 undifferentiated cells were added via a 50 μl suspension in THP-1 growth medium was added to the hydrogel medium along with the SMCs and ox-LDLs. This was followed by an addition of 25 μl of CPPs to the hydrogel. Media changed in the same manner as the CPP ring protocol previously described.

### Creation of Disease Vessels and In Vitro Atherosclerotic Disease Model

Diseased vessels were created from the ND rings only containing ox-LDLs and SMCs. This done via the RSM and in the same method as described earlier. These ND BEBV were cultured for 2 weeks at 37° C. in an incubator in SMC growth medium. Then the intima layer of the ND BEBV was statically and dynamically seeded with HUVECs in the same manner as seen in Chapter 2. After intima seeding the ND BEBV the PSOS+fitting+ND BEBV was placed back in the static seeding plate. Following this 1.5×10⁷ THP-1 (3×150 mm plates) were centrifuged at 130 rpm for 10 minutes then resuspended in 600 μl of THP-1 growth medium. Then add the THP-1 to M1 differentiation reagents: 1 μl of 10 ng/ml phorbol 12-myristate 13-acetate (PMA), 3 ul of 100 μg/ml lipopolysaccharide (LPS), and 24 μl of 20 ng/ml interferon-gamma (IFN-gamma) to the cell suspension (Mor6n-Calvente et al., 2018). Carefully via pipette the THP-1 suspension into the PSOS+fitting+ND vessel. After incubation at 37° C. for 24 hours carefully aspirate the media and cells and prepare another THP-1 suspension in 500 μl THP-1 growth medium+differentiation reagents. This time add 100 μl of CPPs, 1 μl of FGF and 1 μl of PDGF to the cell suspension. Then carefully pipette the THP-1 suspension into the static system. Following this rotate the PSOS+fitting+ND BEBV 120°. After 24 hours of incubation, carefully aspirate the cells and media and carefully pipette another THP-1 suspension in 600 μl THP-1 growth medium+differentiation reagents+150 μl CPPs+1 μl of FGF and PDGF. Rotate another 120° then incubate for at 37° C. for 24 hours. Following this carefully aspirate and prepare ND vessel for histology.

### Tensile Testing

Tensile testing of all in vitro tissue samples was performed on a Cellscale uniaxial stretch device (Cellscale, Montreal, Canada). The strain rate was set to 0.4 mm/s. Samples were loaded into the Cellscale using custom steel hooks. Sandpaper was adhered to a piece of foam to create grip pads which were then attached to the steel hooks. The samples were looped around the hooks before stretching. These grips were used for all groups: normal human vessel (n=1); calcified human vessel (n=1); engineered vascular rings (n=5); CPP-treated rings (n=5); normal engineered vessels (n=6); CPP-treated engineered vessels (n=5); and in vitro EDM rings. Following loading, the initial length, thickness and lumen diameter of each sample was measured using calipers for force, stress and strain calculations. All groups were circumferentially stretched until failure.

### Histology

ND BEBVs and EDM rings were fresh fixed into optimal cutting temperature (OCT) compound, cryosectioned into 10 ml cross-sectional sections, mounted onto slides, and air-dried overnight prior to staining. Samples were stained with hematoxylin and eosin (H&E), Von Kossa Stain and Oil Red O Stain. In H&E stained samples, the purplish structures shown indicate cell nuclei, while pink regions indicate cytoplasm and residual fibrin gel. The Von Kossa stained samples, the nuclei are stained red, the light pink regions indicate the cytoplasm and calcium deposited stain brown black. In the O Red O stained samples, the purplish structures are the nuclei and any oil or lipids present in the sample stain red.

### Statistics

All averages are reported as mean values±standard deviation. ANOVA with Tukey post hoc test was performed for tensile data comparison with EDM rings. Student's T test was run on control vs CPP group tensile and simulation data. Alpha value for significance was set to 0.05.

### Mathematical Model

### Model Geometry and Material Properties

The software package used to create the computational model was ANSYS 19. The geometry for the mathematical model was a straight, three-dimensional cylinder with dimensions based on the engineered vessel size applicable to the clinic. An inlet and outlet were added to the simulation to accurately replicate the flow pattern through the BEBVs in our custom perfusion testing system. The cylinder was set to 42 mm long in the z direction with three tube segments composed of a 15 mm long inlet silicone tube, a 12 mm long BEBV and a 15 mm long outlet silicone tube. Both the silicone tubes had an inner diameter of 5 mm and a thickness of 0.5 mm. The BEBV had a lumen diameter of 5 mm and vessel wall thickness of 1 mm. In the computer model, the silicone tubes were represented as linear anisotropic materials and the BEBV was represented as a hyper-elastic material.

### Numerical Model for Fluid-Structure Interaction (FSI) Simulation

For the computational model, assumptions were set for a laminar, incompressible, Newtonian flow. Thus, the governing equation is a simplified derivation of the Navier-Stokes equation for a velocity profile in the y direction (Eq 4-1) which was integrated by parts to obtain equation (Eq. 4-2):

$$μ \frac{1}{r} \frac{d}{dr} ( r \frac{d}{dr} v ) = \frac{dP}{dy} . \qquad Equation 4 \text{-1}$$

$$v ( r ) = \frac{1}{4 μ} \frac{dP}{dy} ( {r}_{0}^{2} - {r}^{2} ) . \qquad Equation 4 \text{- 2}$$

where r is the radius, dP is the pressure drop across dy, and μ is the viscosity of blood (μ=0.0035 Pa·s). Since flow is equivalent to the product of the cross-sectional area and velocity, flow through the blood vessel can be defined as:

$$Q = 2 π {\int }_{0}^{{r}_{0}} v ( r ) rdr = \frac{π}{8 μ} \frac{dP}{dy} {r}_{0}^{4} . \qquad Equation 4 \text{-3}$$

This equation is the Hagen-Poiseuille equation for fluid flow in a tube which approximates blood flow through an artery. To determine whether the flow is laminar or turbulent, the Reynolds Number of the system needs to be calculated by this equation:

$$R e = \frac{ρ v 2 r}{μ} . \qquad Equation 4 \text{-4}$$

where p is the density of blood (p=1060^{kg}). To calculate the wall shear stress of the BEBV, the equation of continuity (Eq. 4-5) and equation of motion for an incompressible fluid (Eq. 4-6) must be evaluated in a cylindrical space:

$$\frac{\partial ρ}{\partial t} + \frac{1}{r} \frac{\partial ( ρ {rv}_{r} )}{\partial r} + \frac{1}{r} \frac{\partial ( ρ {v}_{θ} )}{\partial θ} + \frac{\partial ( ρ {v}_{y} )}{\partial y} = 0. \\ \qquad Equation 4 \text{-5}$$

$$ρ ( \frac{\partial {v}_{y}}{\partial t} + {v}_{r} \frac{\partial {v}_{y}}{\partial r} + \frac{{v}_{θ}}{r} \frac{\partial {v}_{y}}{\partial θ} + {v}_{y} \frac{\partial {v}_{y}}{\partial y} ) = - \frac{\partial P}{\partial y} - [ \frac{1}{r} \frac{\partial ( r {τ}_{ry} )}{\partial r} + \frac{1}{r} \frac{\partial {τ}_{θ y}}{\partial θ} + \frac{\partial {τ}_{yy}}{\partial z} ] + ρ {g}_{y} . \qquad Equation 4 \text{-6}$$

The arbitrary values go to zero, leaving the simplified equation for the wall shear stress of an artery as:

$${τ}_{ry} = \frac{dPr}{2 y} . \qquad Equation 4 \text{-7}$$

Deformations of the BEBV was modeled using a three-term (Eq. 4-8) and five-term (Eq. 4-9) Mooney-Rivlin equation:

W=C₁₀(Ī₁−3)+C₀₁(Ī₂−3)+C₁₁(Ī₂−3)+D(J−1)² Equation 4-8.

W=C₁₀(Ī₁−3)+C₀₁(Ī₂−3)+C₂₀(Ī₁−3)²+C₀₁(Ī₂−3)²+C₁₁(Ī₁−3)(Ī₂−3)+D(J−1)² Equation 4-9.

where W is the strain energy density; C_{1O}, C_{O1}, C₁₁, C_{2O} are material constants related to distortional response; D is the material constant related to the volumetric response; and Ī₁, Ī₂ are the first and second invariants as described by:

Ī₁=λ₁²+λ₂²+λ₃² Equation 4-10.

Ī₂=λ₁²λ₂²+λ₂²λ₃²+λ₁²λ₃² Equation 4-11.

Ī₃=λ₁²λ₂²λ₃²=J² Equation 4-12.

where iI₁, iI₂, iI₃ are stretch ratios and J is the Jacobian determinant. The experimental uniaxial tensile data for the control and CPP BEBV groups was used to create a curve fit that was solved computationally to obtain the material constants. The radial deformation of the control and CPP BEBVs under normal and occluded states was then compared.

The algorithm for the equations for the fluid, structural and re-meshing factors was solved in a stepwise, coupled sequence. Convergence was determined using the Arbitrary Lagrangian-Eulerian (ALE) formula at each 0.05 s time-step increment for a time period of 1 s. The calculations were performed with respect to time at various distances y along the entire length of the vessel.

### Boundary Conditions

A time-dependent, non-linear velocity profile at the inlet was created based on the flow capabilities of the perstaltic pump (Longer Precision Pump Co., Ltd, Tucson, Ariz.) being used to collect the experimental data:

vᵢₙₗₑₜ=vₚᵤₘₚ[1−cos(4πt)] Equation 4-13.

where v is the flow velocity of the pump (vₚᵤₘₚ=0.0852 m/s). The period of the flow waveform T was 1 s. Fluid velocity in the y direction was zero at the walls of the BEBV and silicone tube, where r=0 at the center of the tube and r=R at the wall. The velocity of the fluid described in Equation 4-2 holds along length y with respect to the radius of the tube. The pressure inlet was set to zero. The pressure at the outlet of the tube was defined as:

$${P}_{outlet} = {P}_{pump} + Δ P . \qquad Equation 4 \text{-14}$$

$$Δ P = - Δ y [ \frac{8 μ {v}_{pump}}{{r}_{vessel}^{2}} ( 1 + \frac{2}{3} cos ( 4 π t ) ) ] . \qquad Equation 4 \text{-15}$$

$${P}_{outlet} = {P}_{pump} = - Δ y [ \frac{8 μ {v}_{pump}}{{r}_{vessel}^{2}} ( 1 + \frac{2}{3} cos ( 4 π t ) ) ] . \qquad Eqution 4 \text{-16}$$

Where Δγ is the length of the vessel (0.042 m), rᵥₑₛₛₑₗ is the radius of the lumen of the vessel (0.0025 m), Pₚᵤₘₚ is the pressure of the peristaltic pump (500 Pa) and the expression for ΔP is derived from a non-linear pressure gradient equation (Chakravarty & Mandal, Math. Comput. Model., 24:43-58, 1996, Zaman et al., PLoS One. 11(8):e0161377, 2016. doi: 10.1371/journal.pone.0161377). The wall shear stress is maximum where r=R and zero where r=0. The BEBV and silicone tube were fixed at both ends.

### Model Validation

The mathematical model was validated using the ANSYS 19 workbench by means of a Computational Fluid Dynamics (CFD) method. A system coupled computational FSI simulation was created via the Transient Structural and Fluent toolboxes. The Transient Structural toolbox validated the Mooney-Rivlin equations (8) and (9) using experimental tensile data for both the control and CPP BEBVs. The Fluent toolbox validated the hemodynamic equations, and the nonlinear derivations for the bioreactor pump inlet velocity and outlet pressure. Those same nonlinear equations for the inlet velocity and outlet pressure were written in C as a script and compiled into a user defined function (UDF) in order to be utilized by the CFD simulation in Fluent. Via toolboxes the mathematical model was run for a period 1 s at 20 timesteps in increments of 0.05 s. Although there are 20 timesteps data will be analyzed at timesteps 0.25 s, 0.5 s, 0.75 s and 1 s. Timesteps 0.25 s and 7.5 s will represent the peak values due to those times being the highest values inside the period. Also, timesteps 0.5 s and 1.0 s represent the lower values due to those times being the lowest values inside with the period.

### Results

### Establishment of the BEBV 3D Computer Model and Tissue Model

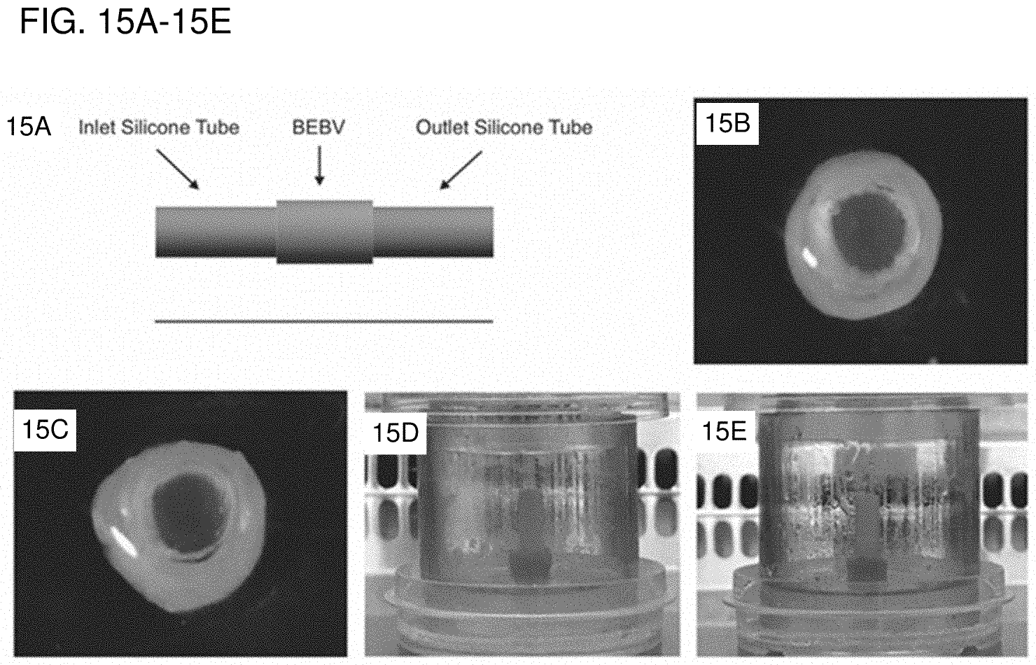

A diagram of the 3-dimensional computer model is provided in FIG. 15A. A 3D virtual representation of the silicone inlet and outlet tubes as well as the BEBV were created using the 3-dimensional software SpaceClaim (SapceClaim Corporation, Concord, Mass.). Smooth muscle cell tissue engineered rings were able to form successfully with the addition of CPPs and is shown alongside control rings without CPPs (FIGS. 15B and 15C). Both control and CPP groups were successfully stacked into BEBVs in tall plates (FIGS. 15D and 15E). The addition of CPPs into the BEBVs did not change the macroscopic appearance compared to control engineered vessels.

### CPP Calcification Significantly Affects Engineered Vascular Ring Mechanical Properties

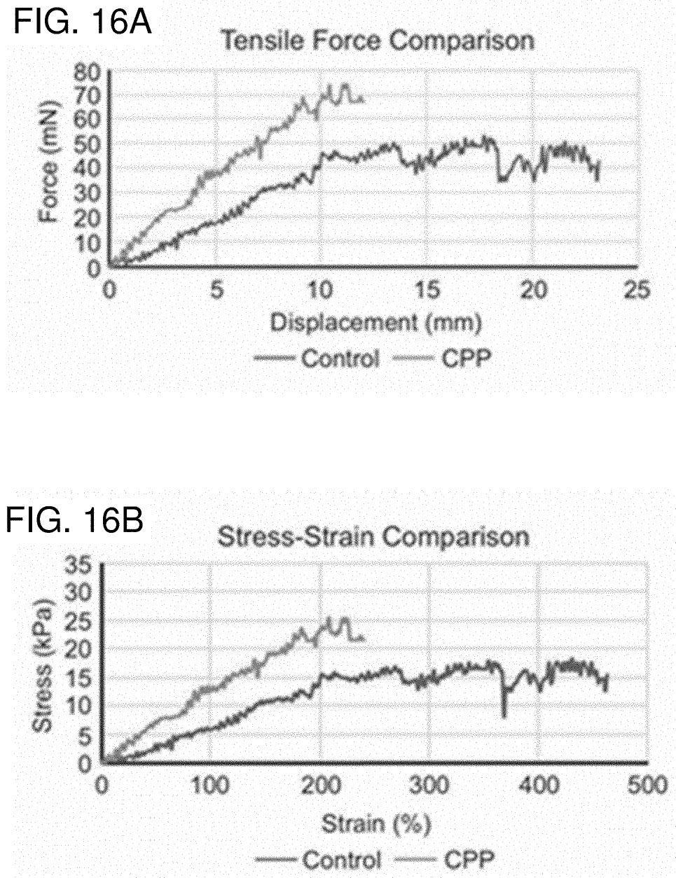

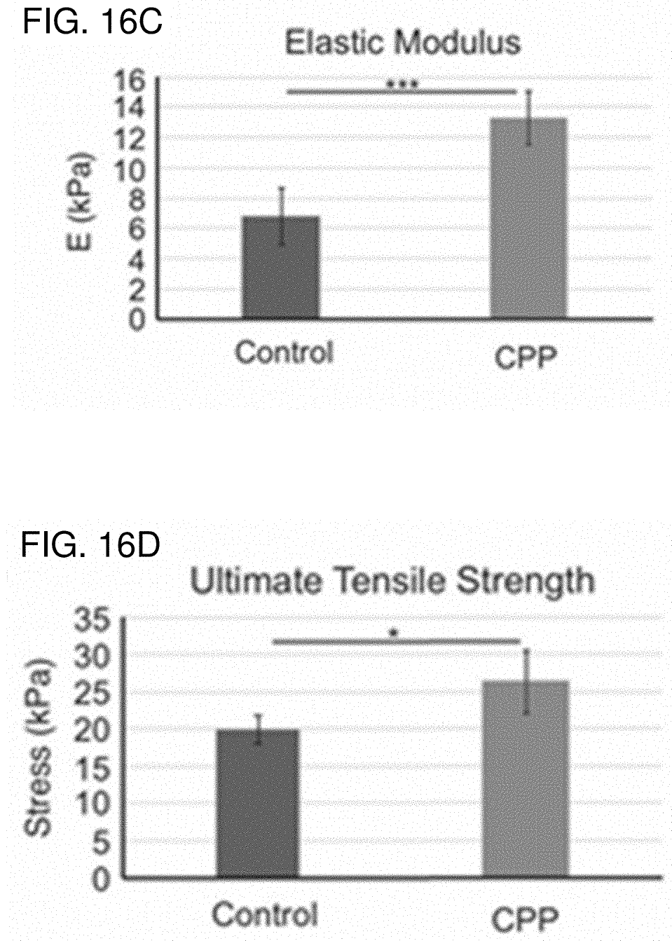

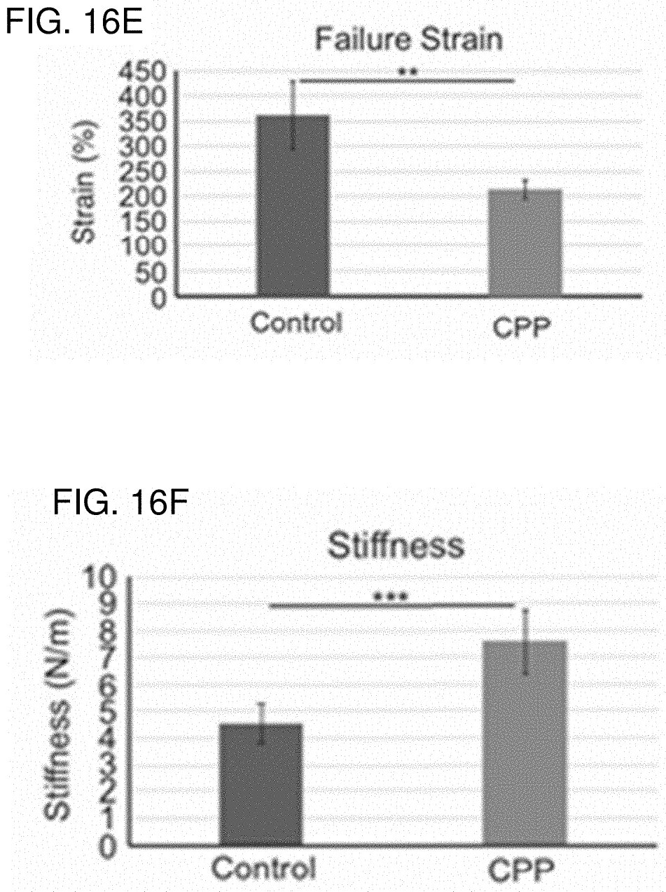

Tensile results from the control (n=5) and CPP (n=5) tissue rings are shown in FIGS. 16A-16F. Control and CPP group tensile data comparison is provided in FIG. 16A, where the peak force is 59.8±8.64 mN and 76.6±10.1 mN respectively. To obtain stress-strain curves, applied force was divided by the cross-sectional areas for the control and CPP tissue rings, which were 3.02±0.200 mm² and 2.92±0.133 mm², respectively (FIG. 16B). A comparison of the elastic modulus is exhibited in FIG. 16C. The elastic modulus was 6.71±1.88 kPa for the control group and 13.3±1.81 kPa for the CPP group, which showed statistical significance (p<0.001). A comparison of the ultimate tensile strength of both groups is shown in FIG. 16D. Addition of the CPPs, i.e. calcification of the engineered vascular tissue, significantly increased ultimate tensile strength from 20.0±1.86 kPa in the control group to 26.3±4.19 kPa in the CPP group (p<0.05). A comparison of the strain at failure is provided in FIG. 16E. The strain at failure was 362±66.8% for the control group and 214±18.0% for the CPP group, resulting in a significant decrease (p<0.01) in the strain at failure for the CPP group compared to the control. In FIG. 16F a comparison of the stiffness is shown. The stiffness was 4.55±0.74 N/m for the control group and 7.56±1.16 N/m for the CPP group. The mechanical data showed that addition of CPPs to the engineered vascular rings significantly increased elastic modulus and tensile strength, and decreased failure strength, which are indicative of a calcified state.

### CPP Calcification Significantly Affects BEBV Mechanical Properties

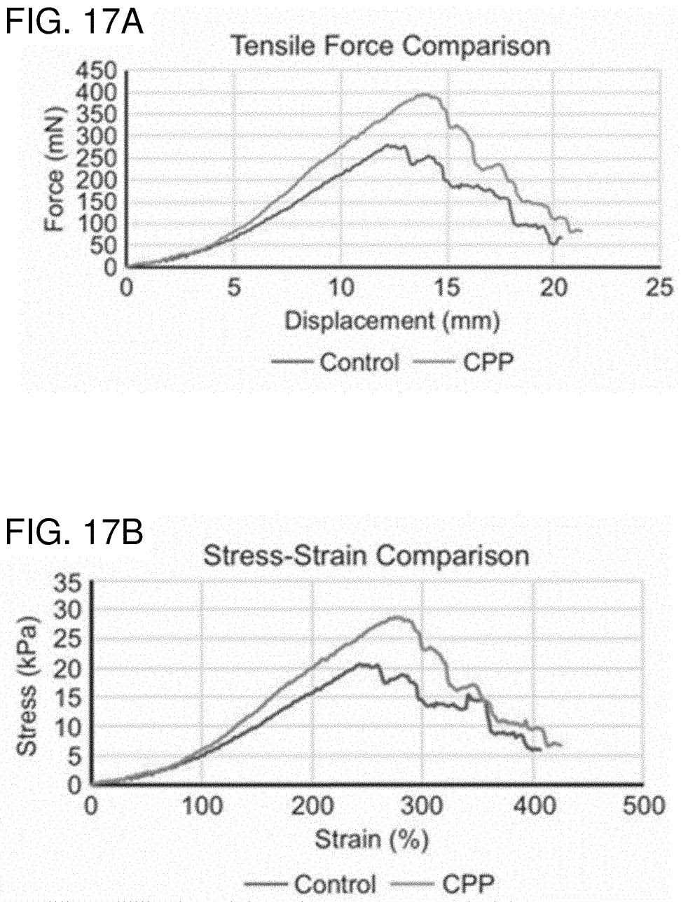

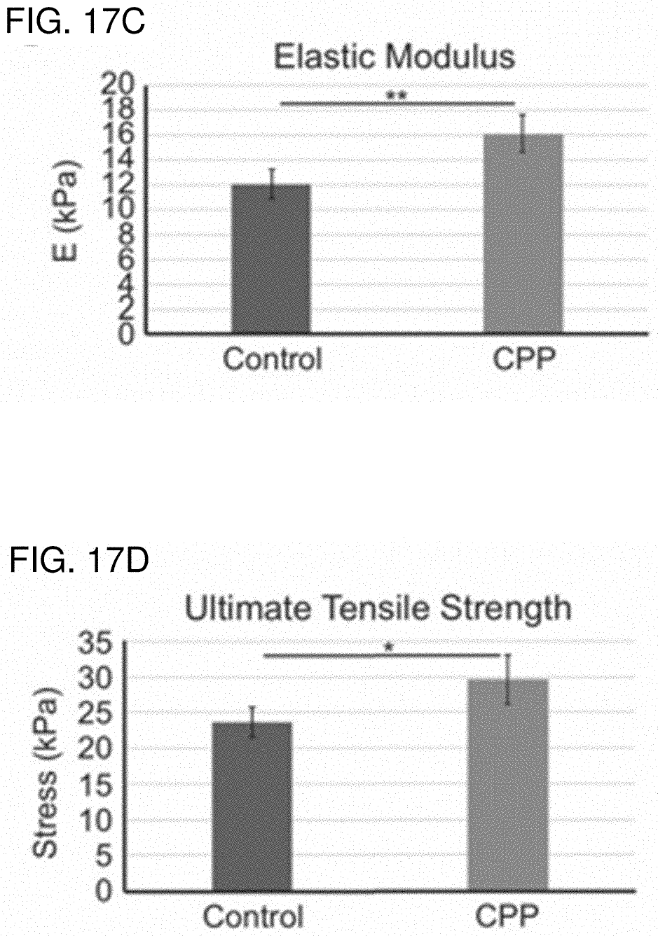

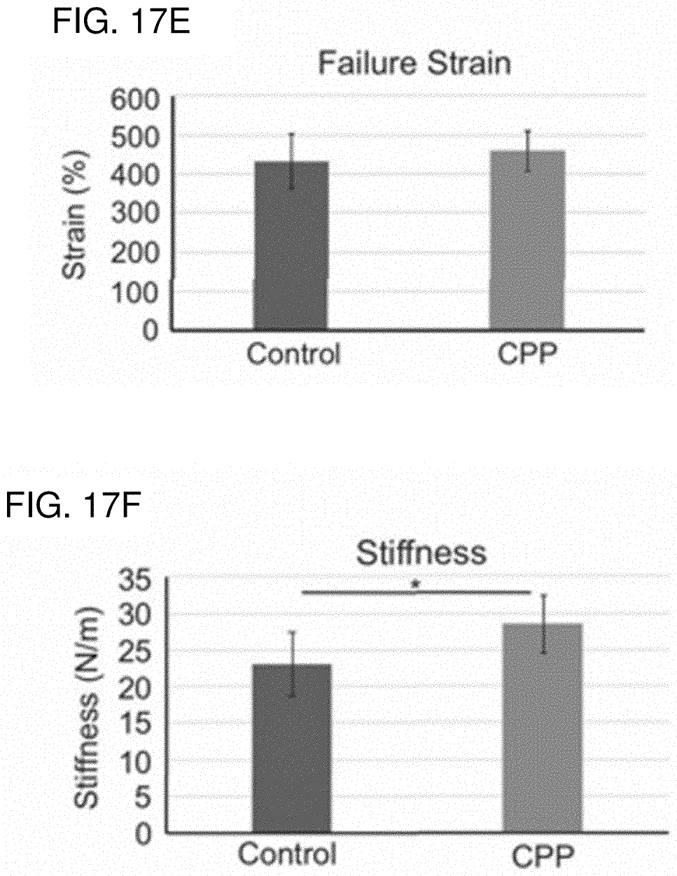

Tensile results from the control (n=6) and CPP (n=5) BEBVs are shown in FIGS. 17A-17F. Control and CPP group tensile data is shown in FIG. 17A, with the peak force as 322±32.0 mN and 406±48.0 mN respectively. Average cross-sectional areas for the control and CPP BEBVs were 13.7±0.330 mm² and 13.8±0.130 mm², respectively. The associated stress-strain curves from the force and cross-sectional area data is shown in FIG. 17B. The elastic modulus was 12.1±1.23 kPa for the control group and 16.2±1.56 kPa for the CPP group (FIG. 17C). These results showed a significant increase (p<0.001) in elastic modulus for the CPP group compared to the control (FIG. 17D). The ultimate tensile strength was 23.6±2.15 kPa for the control group and 29.6±3.40 kPa for the CPP group (FIG. 17E). These results showed a significant increase (p<0.01) in the ultimate tensile strength by the addition of CPPs and hence calcification. The strain at failure was 425±71.0% for the control group and 460±51.9% for the CPP group, which showed no significant change in the strain at failure between groups. In FIG. 17F a comparison of the stiffness is displayed. The stiffness was 23.07±4.33 N/m for the control group and 28.43±3.95 for the CPP group. The mechanical data showed that the CPPs were successful in modifying the engineered vessels (BEBVs) tensile properties indicative of calcification.

### Normal and Diseased Human Common Iliac Artery Mechanical Properties

Human artery tensile mechanics serves as a method to compare whether our stiffness trends observed after the addition of calci-protein particles (CPPs) into our engineered tissues are appropriate. In one study the elastic modulus of common carotid arteries (CCA) from three healthy individuals was measured in vivo (Khamdaeng et al., Ultrasonics. 52(3):402-11, 2012). The in vivo elastic moduli values were 150±40 kPa, 890±270 kPa, and 750±kPa. Brachial blood pressure was measured from the individuals using a sphygmomanometer and the CCA pressure waveform was obtained using a sensor perpendicular to the CCA. They were able to calculate the circumferential stress and strain using experimental values of the inner pressure and diameter of the CCA wall. A study applied a cyclic method of tensile testing analysis where healthy common iliac artery (CIA) samples were preconditioned with a load of 0.05 N for ten cycles (Faturechi et al., Acta Bioeng Biomech. 21(3):13-21. 2019). Then the tissues were stretched 57 failure with a stain rate of 1%/min. Another study extracted healthy (n=8) and atherosclerotic (n=8) human coronary arteries (CA) from deceased patients ranging from different ages and conditions (Karimi et al., 2016). They found that in both circumferential and axial stretch directions the elastic modulus and maximum stress of the atherosclerotic group was significantly higher than the healthy group (p<0.005). This study underwent tensile testing most identical to our own therefore we will compare it to our human common iliac artery tensile data.

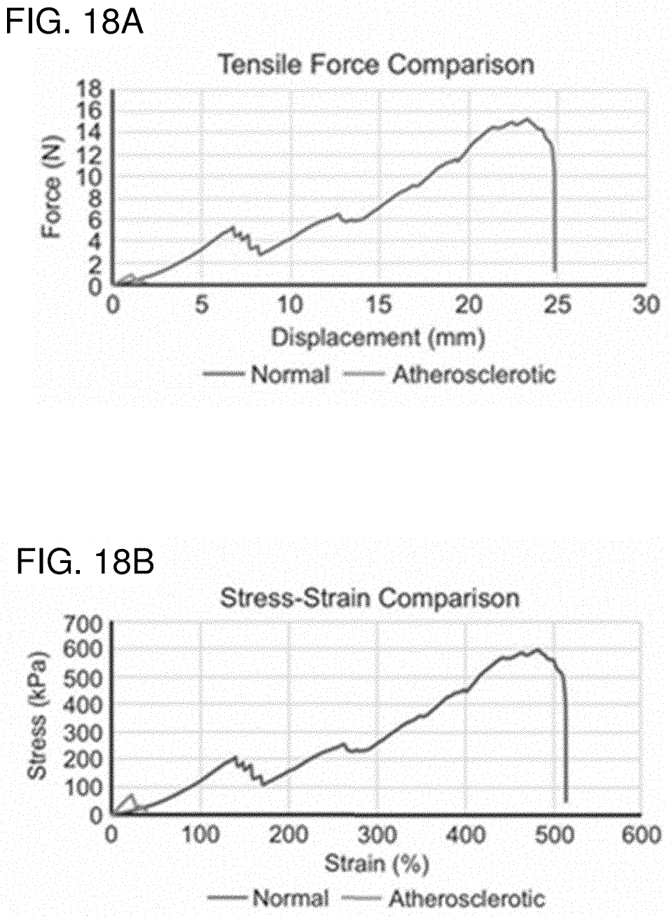

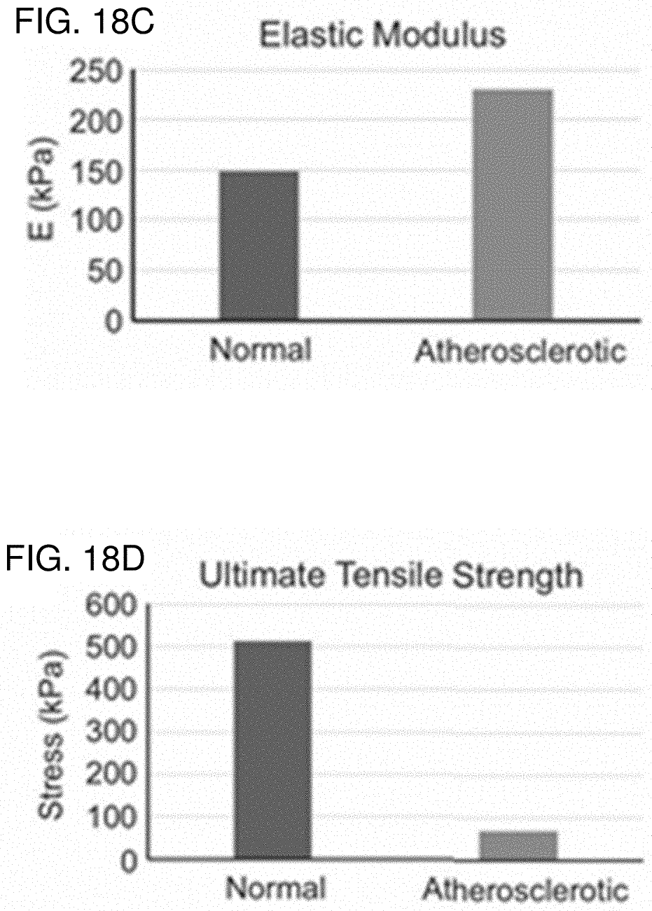

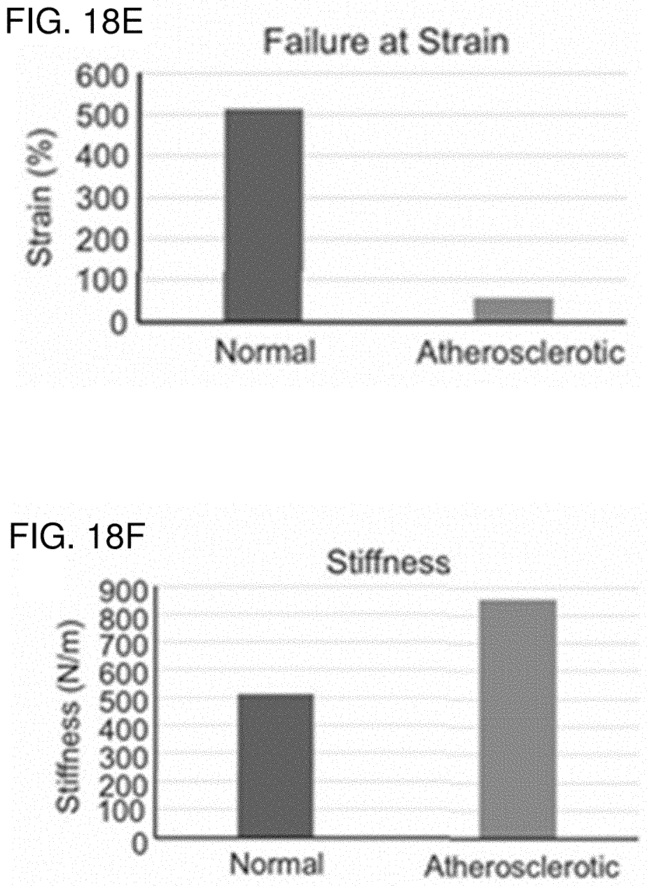

Tensile testing results for human common iliac artery (CIA) in normal (n=1) and atherosclerotic (n=1) states are shown in FIG. 18A-18F. The elastic modulus was 148 kPa for the normal CIA and is 230 kPa for the atherosclerotic CIA (FIG. 18C). These results show an increase in the elastic modulus of the atherosclerotic CIA compared to the normal CIA. When comparing to the elastic moduli from Karimi et al. (Bioengineered 8(2):154-170, 2017), we noticed our values were lower, but the trend of the atherosclerotic group having greater value was the same. The ultimate tensile strength was 598 kPa for a normal CIA and 70.3 kPa for the atherosclerotic CIA (FIG. 18D). These results show that the ultimate tensile strength of an atherosclerotic CIA is about 8.5 times lower that of a normal CIA. On the other hand, in Karimi et al., we notice the ultimate tensile stress for the atherosclerotic group was larger almost 4 times larger than the healthy group. This could be due to either the severity of the CIA we tested compared to the CA the study tested. Another reason could be the way the samples were extracted. Karimi et al., perhaps had higher quality and precision surgical equipment which led to them keeping the tissues and calcification in intact exceptionally. Also, to note the size of the CA could have been different from the CIA we utilized, but the manuscript does not list the nor state this information. On a whole after comparing the two data it appears that human atherosclerotic tissue has a trend of having a higher elastic modulus compared to normal or healthy tissue.

### Computer Simulated BEBV Hemodynamic Properties

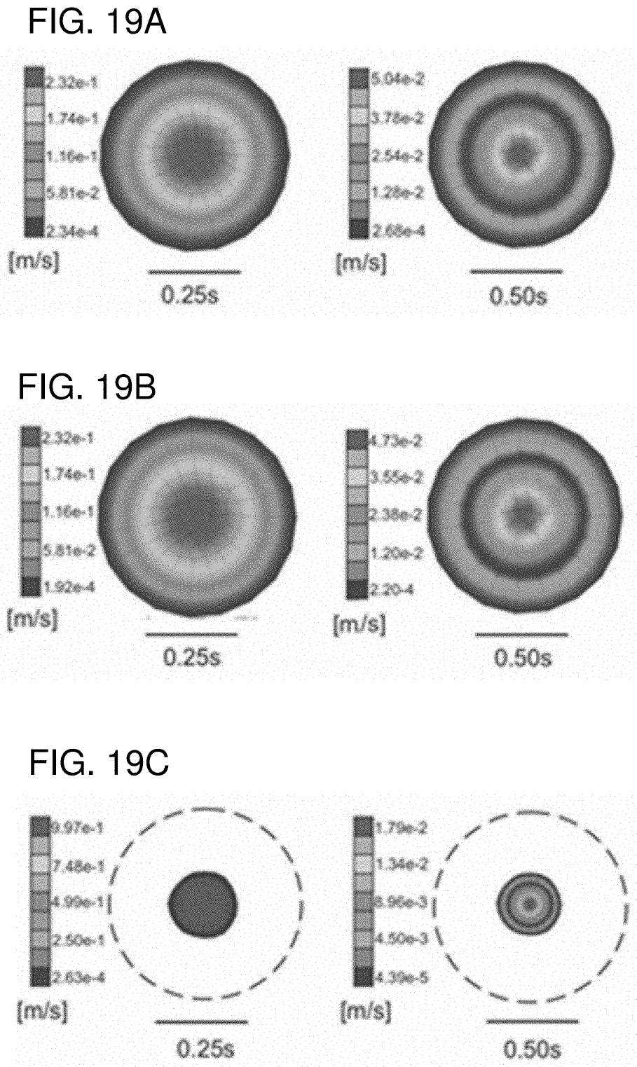

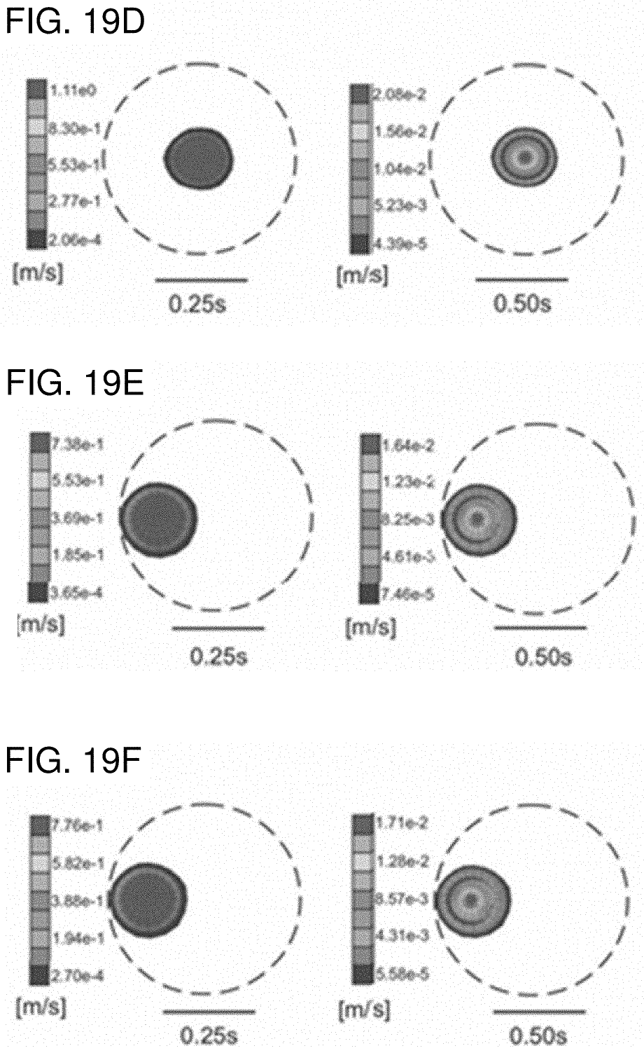

A simulation on flow velocity through the control and CPP BEBVs was created using our laboratory's perfusion bioreactor as a basis to normalize experimental and simulation parameters (FIGS. 19A-19F). The normal control and CPP group velocity profiles approximated at the center of the BEBV were obtained at timesteps 0.25 s, and 0.5 s (FIGS. 19A and 19B). Profiles of both groups were laminar with maximum and larger velocities toward the center of the BEBVs. Results at 0.25 s represent the peak velocity of non-linear pulsatile flow, which is apparent due to the larger number of the higher velocities (FIGS. 19A-19F, red regions) in the center of the BEBVs. Alternatively, 0.5 s represent the lower velocity of the non-linear pulsatile flow, also noticeable due to the lower number of higher velocities. Concentric occlusion of the control and CPP group velocity profiles at the center of the BEBV were simulated (FIGS. 19C and 19D).

The profile radius of the concentric state is severely reduced while the lumen remains center. When observing profiles at 0.25 s, the flow appears less laminar due to the overabundance of high velocities. Results at the lower timestep appear to be laminar due to the reduction of flow. Eccentric occlusion of the control and CPP group velocity profiles at the center of the BEBV were observed at the same timesteps as the normal state (FIGS. 19E and 19F). The profile radius of the eccentric state is severely reduced with the lumen shifted left from the center. When observing profiles at 0.25 s the flow appears less laminar due to the overabundance of high velocities. Results from the lower timestep appear to be laminar due to the reduction of flow.

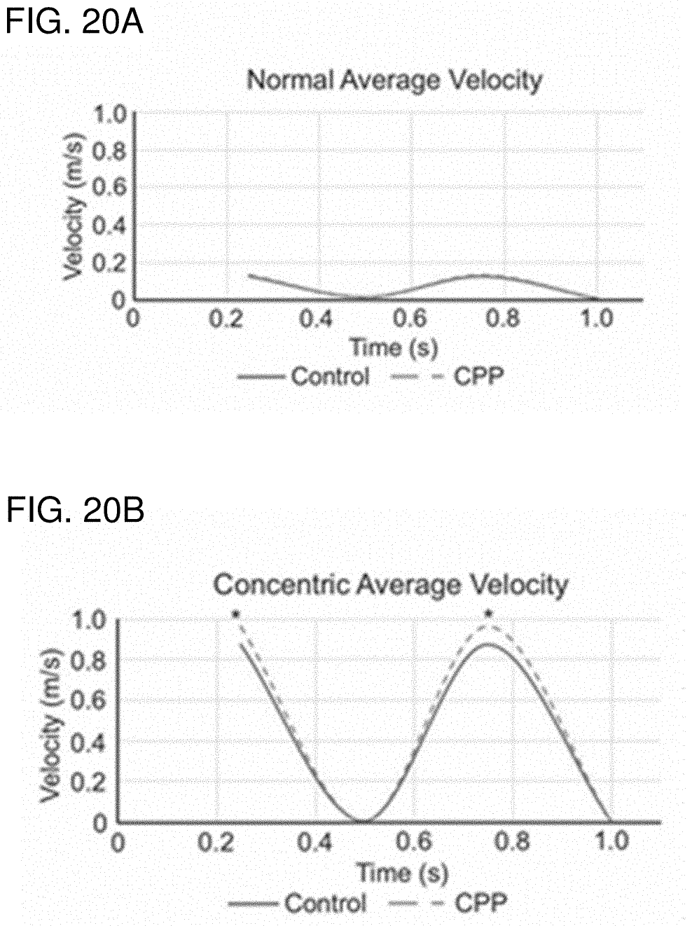

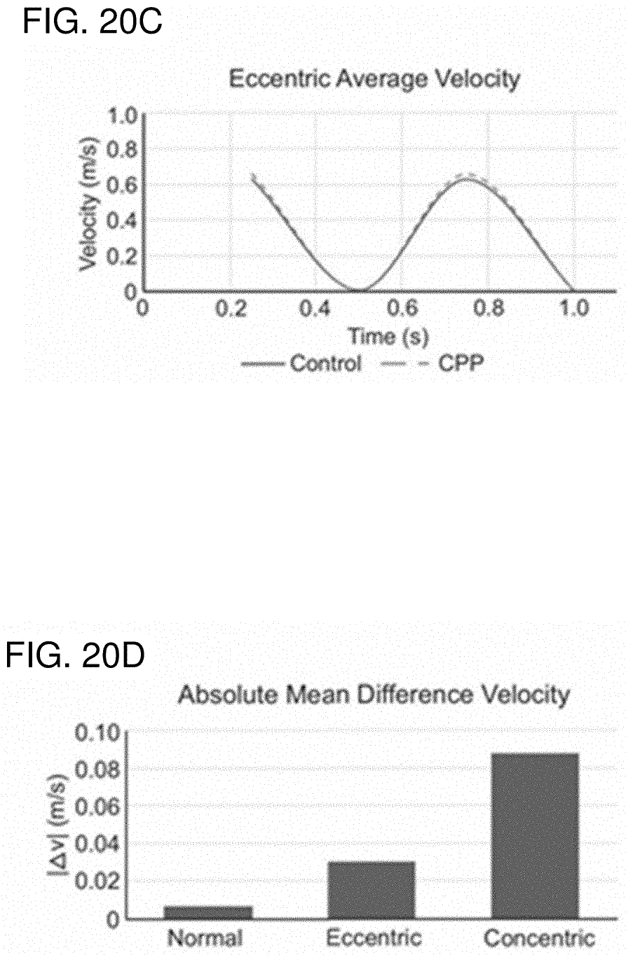

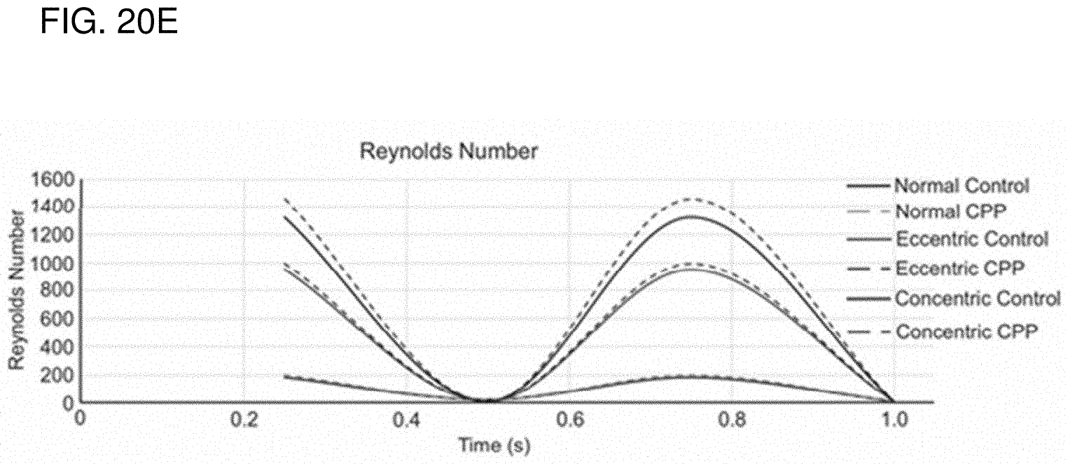

A graph of the average velocities at timesteps 0.25 s, 0.5 s, 0.75 s, and 1 s for the normal state control and CPP groups is shown in FIG. 20A. Lower velocities (at 0.5 s and 1 s) were similar with no significant difference. Peak velocities (at 0.25 s and 0.75 s) are slightly higher for the CPP group but with no significance. Average velocities at the same timesteps for the concentric occluded state control and CPP groups showed that lower velocities were slightly higher for the CPP group but without a significant difference (FIG. 20B). On the other hand, the peak velocities for the CPP group showed a significant increase in velocity (p<0.05). Average velocities at the same timesteps for the eccentric occluded state control and CPP groups are shown in FIG. 20C. peak velocities were slightly higher for the CPP group compared to the normal group but without any significant difference. The peak velocities for the CPP group compared to the normal group were higher with no significant difference in velocity. A comparison of the absolute mean peak velocity differences of the normal and occluded states is shown in FIG. 20D. The absolute mean peak velocity difference is 0.00667 m/s for the normal state, 0.0300 m/s for the eccentric occluded state and 0.0879 m/s for the concentric occluded state. These results suggest that CPP-induced calcification of arterial walls plays a role in increasing blood flow velocities at the site of occlusion.

The Reynolds number describes the flow pattern, with lower numbers indicating laminar flow and higher numbers indicating turbulent flow. The Reynolds number for the normal and occluded states were evaluated (FIG. 20E). Results of the Reynolds number versus time analysis showed that there was little to no difference in the Reynolds number at the peak timesteps for both groups in the normal state. In contrast, for the occluded states, there was an increase in the Reynolds number for the CPP group compared to the control group at the peak timesteps, with the concentric occlusion group displaying the largest Reynolds number. The Reynolds number for the normal and occluded states describe a flow that is laminar. Due to the increase in velocity at the site of occlusion, the Reynolds number has increased showing that the flow will transition into turbulent flow as the plaque grows and the vessel wall further stiffen. This analysis shows that CPP calcification increases the Reynolds number in the presence of an occlusion.

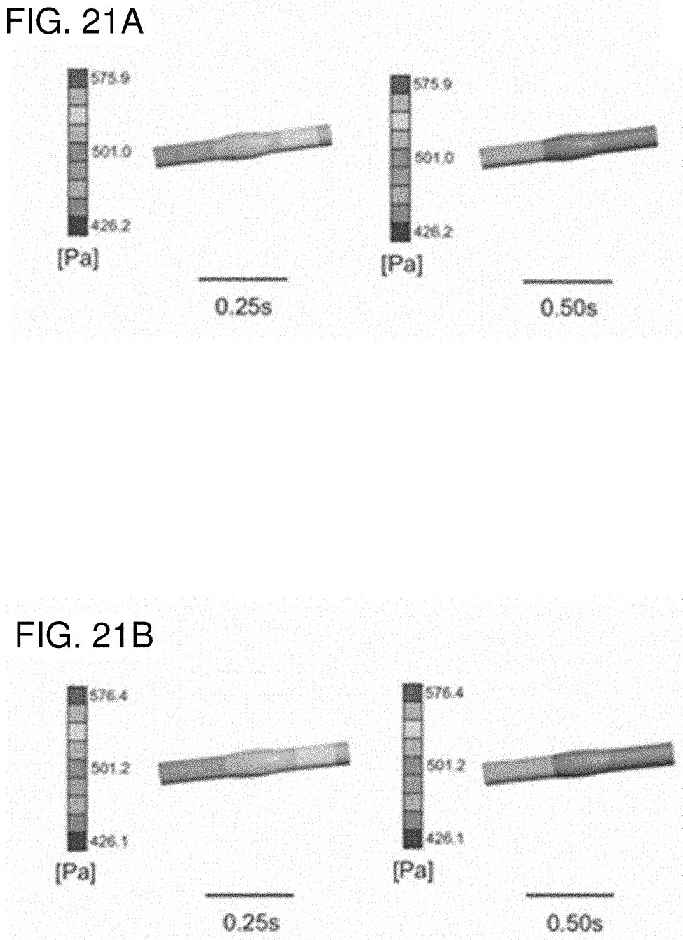

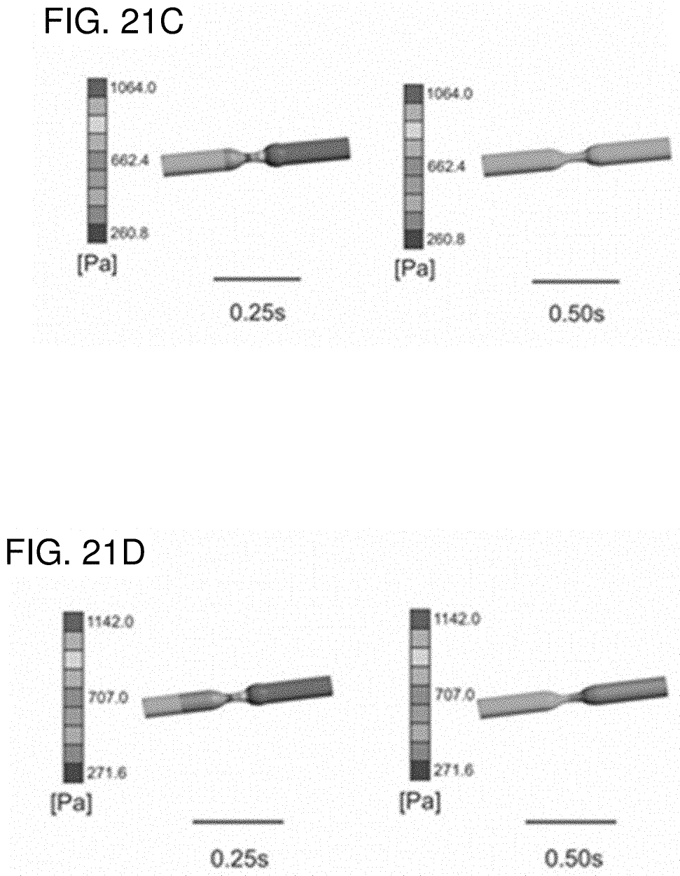

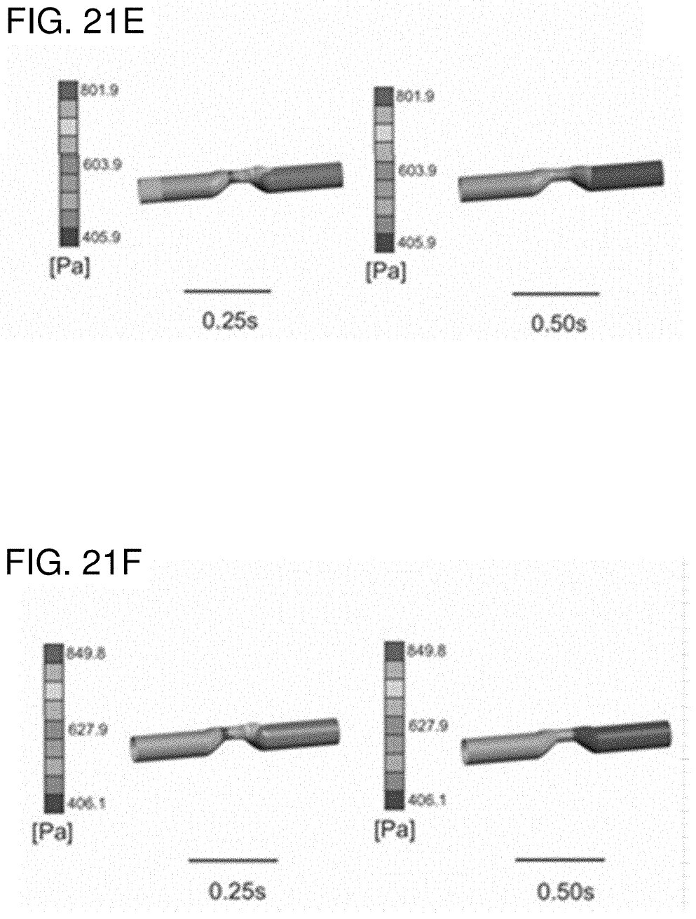

Simulated bioreactor blood pressure results are displayed in FIGS. 21A-21F. The normal state control and CPP group pressure gradient along the length of the modeled bioreactor setup were observed at timesteps 0.25 s, and 0.5 s (FIGS. 21A and 21B). Both groups showed an increase in pressure inside the BEBV and silicone tubes at the peak timestep of 0.25 s where pressure was maximized. At the lower timestep of 0.5 s, the pressure was minimized. For the concentric occlusion state control and CPP groups, a significant increase in pressure at and before the modeled occlusion site during the peak timestep at 0.25 s was observed, with the concentric occluded CPP BEBV model displaying the highest pressures (FIGS. 21C and 21D).

Lower pressures are seen at timestep 0.5 s for both concentric occluded states. For the eccentric occlusion state control and CPP groups, a significant increase in pressure before and at the occlusion site during the peak timestep at 0.25 s was observed, with the eccentric occluded CPP BEBV model displaying the highest pressures (FIGS. 21E and 21F).

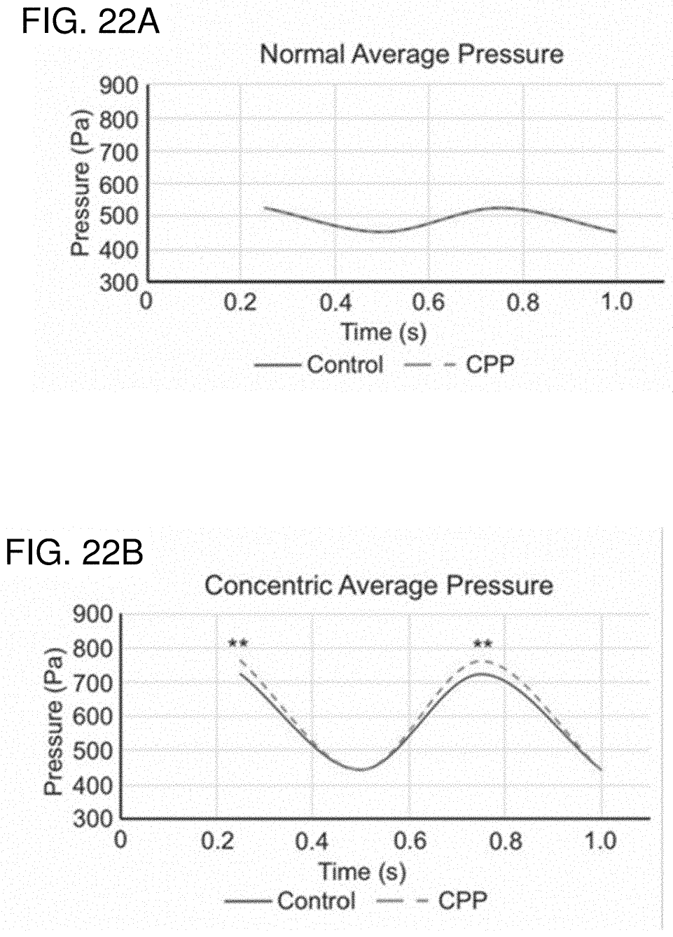

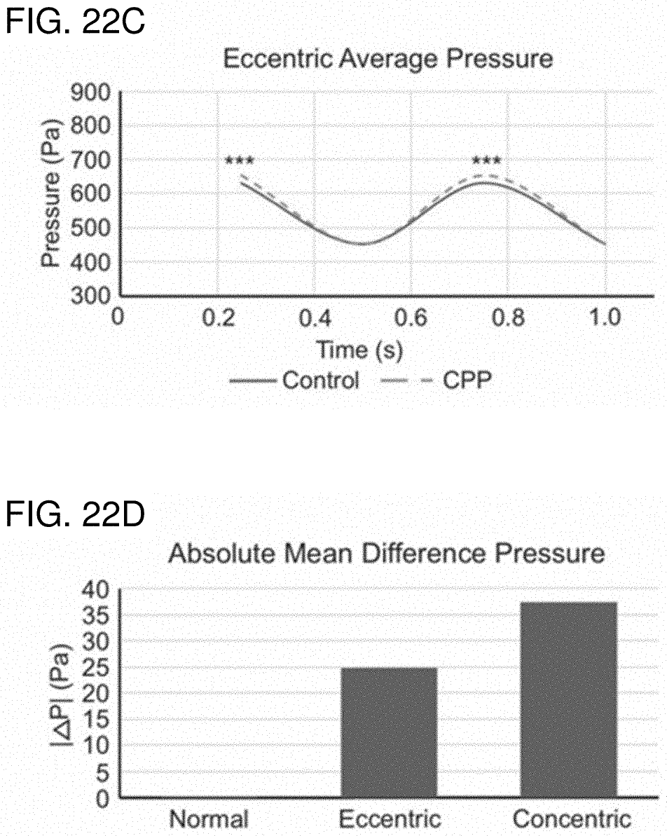

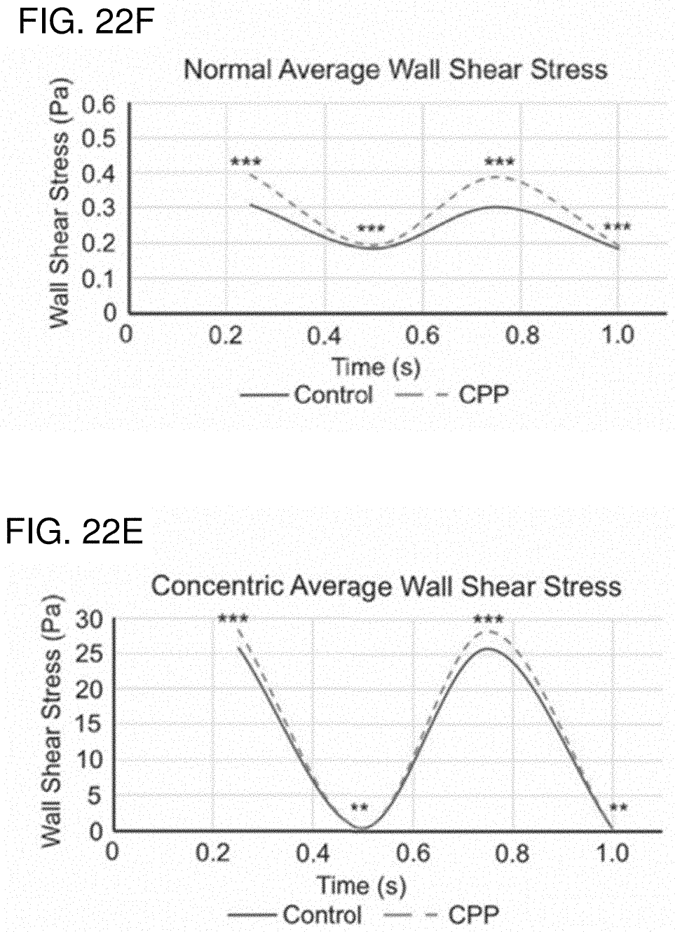

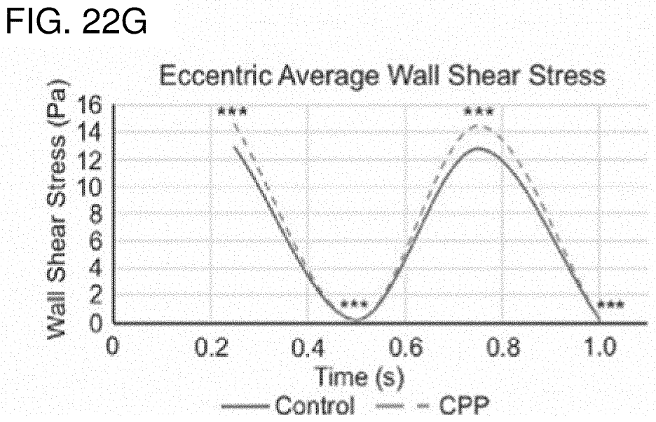

The average pressure values at timesteps 0.25 s, 0.5 s, 0.75 s and 1 s for the normal state control and CPP group are shown in FIG. 22A. Minimum pressures (at 0.5 s and 1 s) were similar with no significant difference between the groups. Peak pressures (at 0.25 s and 0.75 s) also were similar with no significance. Concentric occluded state pressures displayed no significant difference between the groups for lower pressures (FIG. 22B). For peak pressures in the concentric occluded state, the CPP group displayed a significant increase in pressure compared to the control group (p<0.01). For the eccentric occluded state control and CPP groups, lower pressures appeared similar with no significant difference between the groups (FIG. 22C). On the other hand, the peak pressures for the CPP group displayed a significant increase in pressure compared to the control group (p<0.001). The absolute mean peak pressure difference was 0.253 Pa for the normal state, 37.5 Pa for the concentric occluded state and 24.7 Pa for the eccentric occluded state (FIG. 22D). These results show that CPP calcification of the arterial vessel wall increases the fluid (i.e. blood) internal pressure before and at the occlusion site for both concentric and eccentric plaque formations.

The average wall shear stress was analyzed of the normal state control and CPP BEBV at the center of the vessels as mounted in the bioreactor setup (FIG. 22E). The wall shear stress for the CPP group was significantly higher than the control group across all timesteps (p<0.001). The wall shear stress was significantly higher in the CPP group across all timesteps compared to the control group in both the concentric (p<0.001) and eccentric (p<0.01) states (FIGS. 22F and 22G). These results show that CPP-induced calcification affected the tangential stress of the blood flow at the vessel walls.

### Computer Simulated BEBV Vessel Wall Mechanics

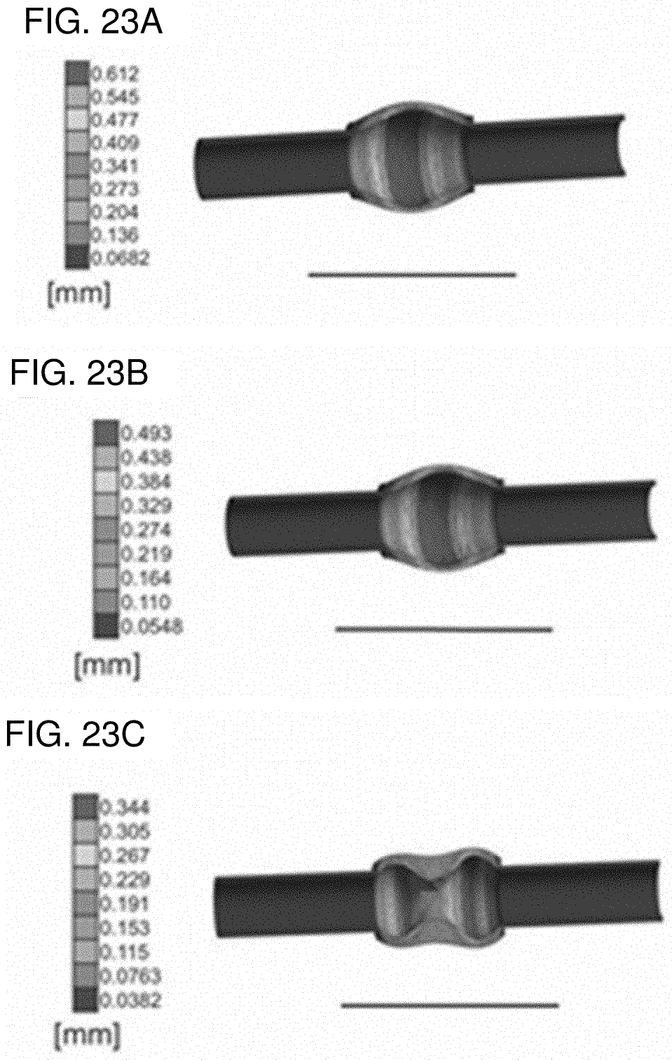

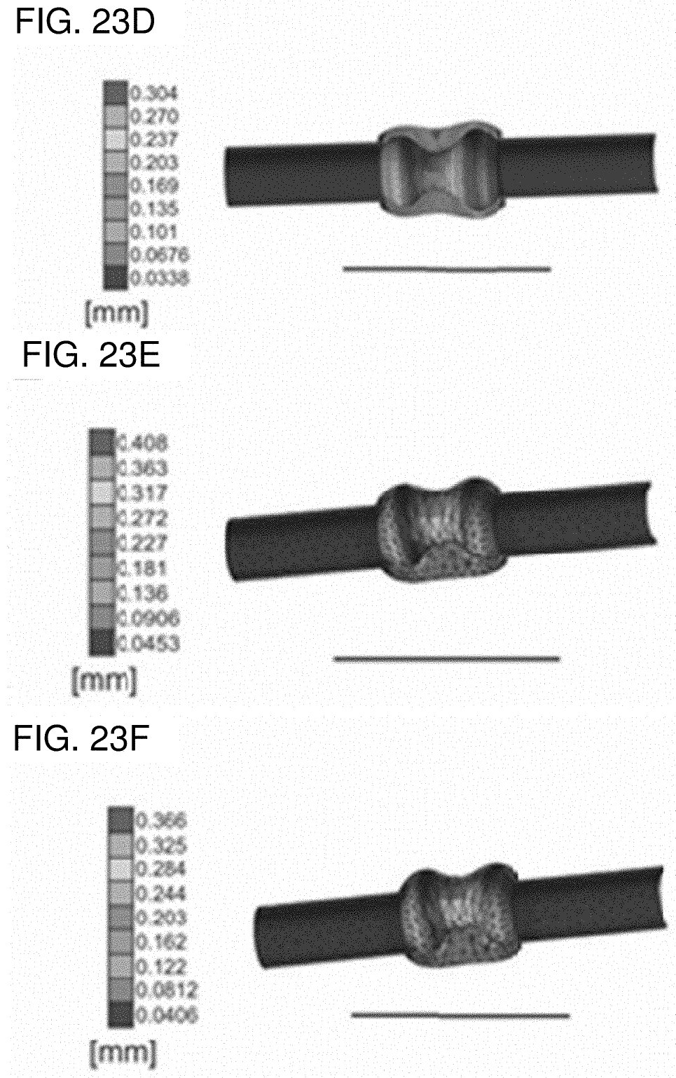

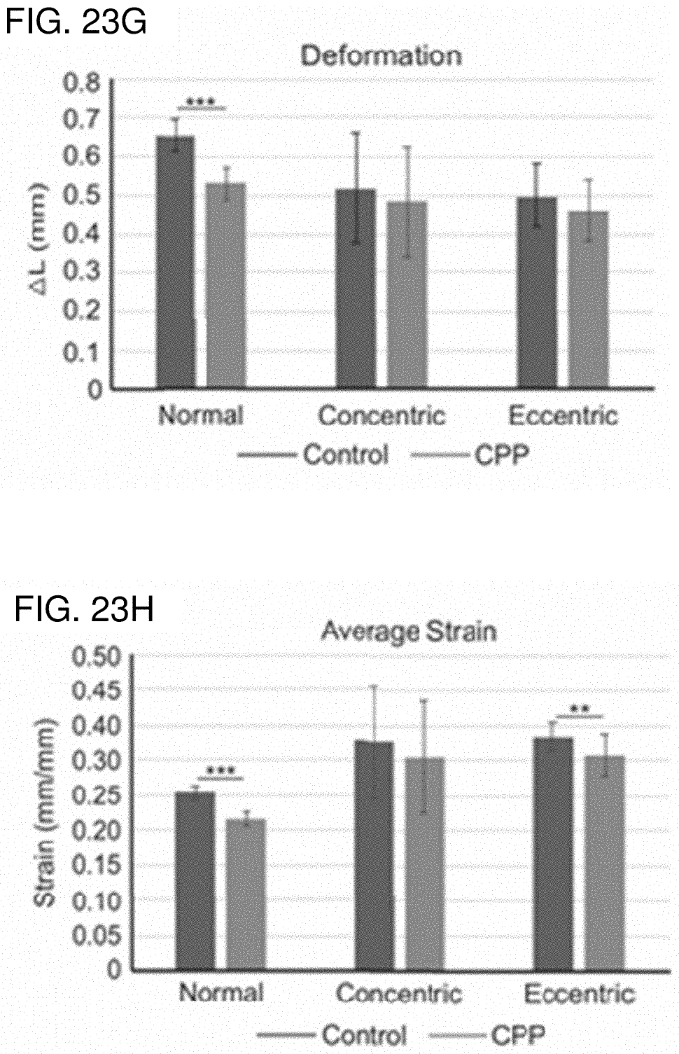

Vessel wall deformation for normal, concentric occluded and eccentric occluded state BEBVs were evaluated in the simulation (FIG. 23G). Average deformation over timesteps from 0.05 s through 1 s in 0.05 s increments was evaluated. The normal state had an average deformation of 0.653±0.0405 mm for the control group and 0.530±0.0366 for the CPP group. Interestingly, the CPP group displayed a significant decrease in the average deformation compared to the control group (p<0.001). The concentric occluded state had an average deformation of 0.519±0.139 mm for the control group and 0.484±0.153 mm for the CPP group. There was a slight decrease in the CPP group compared to the control group although with no significance. The eccentric occluded state had an average deformation of 0.498±0.0750 mm for the control group and 0.462±0.0790 for the CPP group. There also was a slight decrease in the CPP group compared to the control group but with no significance. These results suggest, similarly to the tensile data, that CPP induction lowers the compliancy with a greater significance when plaques are not present. When plaques are present compliancy is already impacted by the thickening of the vessel walls but the addition of CPPs still slightly stiffens in the occluded states.

The vessel wall strain for normal, concentric occluded and eccentric occluded state BEBVs were evaluated in the simulation (FIG. 23H). Average strain over timesteps from 0.05 s through 1 s in 0.05 s increments was evaluated. The normal state had an average strain values of 0.254±0.0140 mm/mm for the control group and 0.217±0.0100 mm/mm for the CPP group (FIG. 23H), representing a significant decrease in the CPP group compared to the control group (p<0.001). These results suggest, similarly to the tensile data, that the CPPs lowered the compliancy of the BEBVs due to calcification of the vessel wall. The concentric occluded state had an average strain of 0.326±0.0760 mm/mm for the control group and 0.306±0.0790 mm/mm for the CPP group. There was a slight decrease in strain in the CPP group compared to the control group although with no significant difference. The eccentric occluded state had an average strain of 0.334±0.0240 mm/mm for the control group and 0.308±0.0280 mm/mm for the CPP group. Similar to the normal state, there is a significant decrease in the CPP group compared to the control group (p<0.01). These results suggest that the increase in pressure before and at the occlusion sites for both concentric and eccentric plaque formation is increasing the strain the vessel wall experiences.

Vessel wall stress for normal, concentric occluded and eccentric occluded state BEBVs were evaluated in the simulation (FIG. 23I). Average stress over timesteps from 0.05 s through 1 s in 0.05 s increments was evaluated. The normal state had an average stress of 1760±129 Pa for the control group and 1740±126 Pa for the CPP group, with no significant difference between the groups. The concentric occluded state had an average stress of 1787±345 Pa for the control group and 1840±388 Pa for the CPP group. The values for the CPP group were slightly increased compared to that of the control but with no significance. The eccentric occluded state had an average stress of 1660±208 Pa for the control group and 1690±234 Pa for the CPP group. Average stress value for the CPP group was slightly increased compared to the control but with no significance. These results show that the addition of CPPs to induce calcification to a concentrically and eccentrically occluded vessels results in a significant increase in pressure, leading to an increase in stress on the arterial wall.

### Tensile Mechanics of the Engineered Disease Media Ring

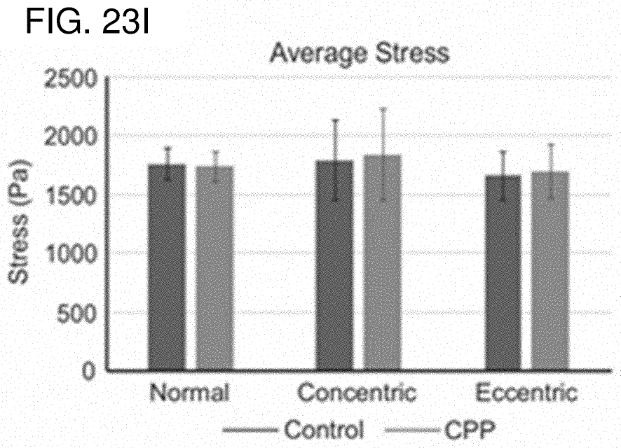

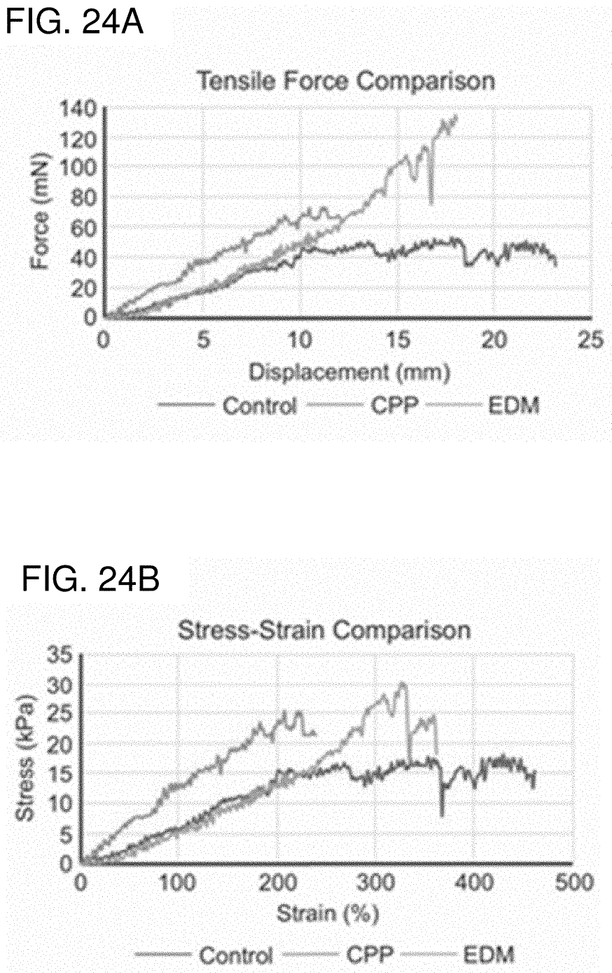

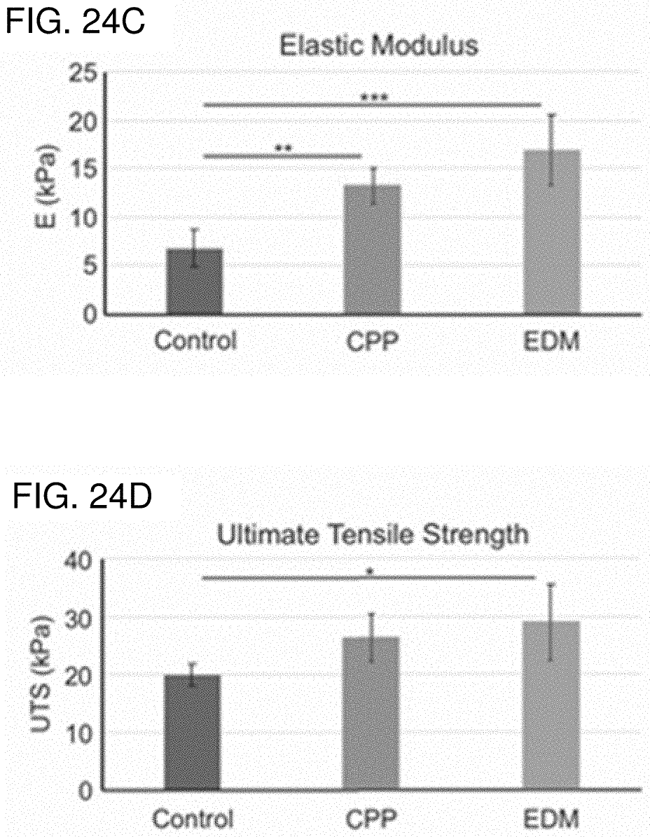

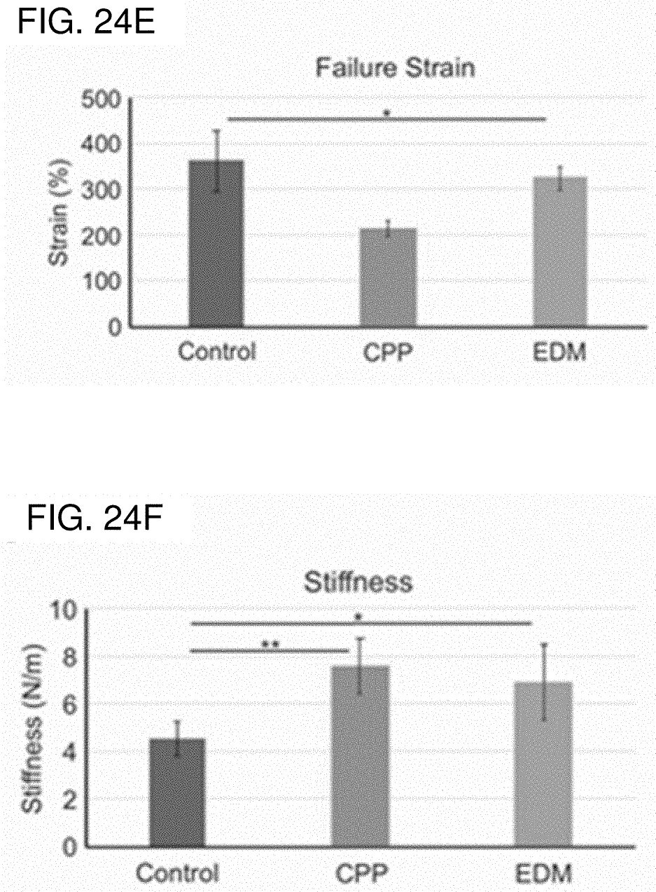

EDM ring tensile mechanics were compared to the control rings and CPP rings reported on earlier in the chapter. Tensile force comparison graphs are shown in the in FIG. 24A, where the EDM had average peak force of about of 91±27 mN. A stress-strain comparison of is shown where tensile properties are seen to differ from that of the control and CPP rings (FIG. 24B). The EDM ring had an elastic modulus of 16.9±3.6 kPa (FIG. 24C), which was significantly higher (p<0.001) than that of the control ring. Observing the other tensile properties, the EDM had ultimate tensile strength of 29.0±6.55 kPa (FIG. 24D), a failure strain of 324±25.3% (FIG. 24E) and a stiffness of 6.88±1.60 N/m (FIG. 24F). All these values were significantly different (p<0.05) from the control group showing a change in mechanical properties due to the macrophages, oxLDL and CPPs present in the tissues. On the other hand, these values were not significantly different from the CPP group.

### Engineered Disease Media Ring Histology

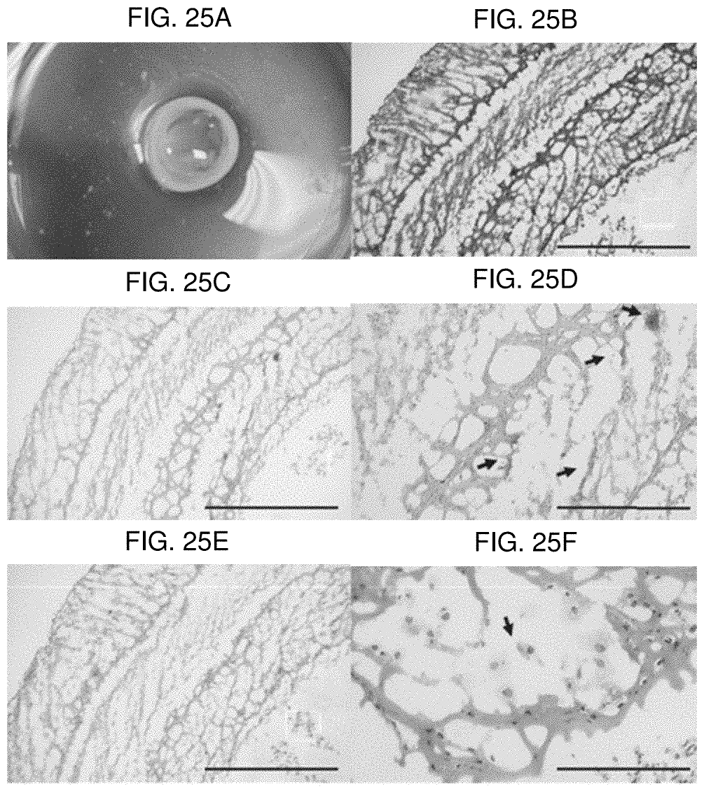

FIG. 25A shows an image of an EDM ring that has successfully rolled in towards the post, forming a stable tissue. An H&E stain was performed on the EDM ring to determine whether cells were still present in the tissue (FIG. 25B). Following this a Von Kossa stain was conducted to probe for the presence of the CPPs (FIGS. 25C and 250). The CPPs stained brown black (black arrows), due to exposure of silver nitrate to UV light during a step in the staining process and can be observed embedded in the hydrogel. An Oil Red O stain was performed (FIG. 25E) the hydrogel stained red due to the abundance of ox-LDLs embedded inside. FIG. 25F is a close up of a THP-1 monocyte that has some ox-LDLs that were present in the hydrogel (black arrow). This assumed because a part of cell stained red and only lipids stain that color in this method.

Early Stage In Vitro Atherosclerosis Disease Model with Natural Disease BEBV

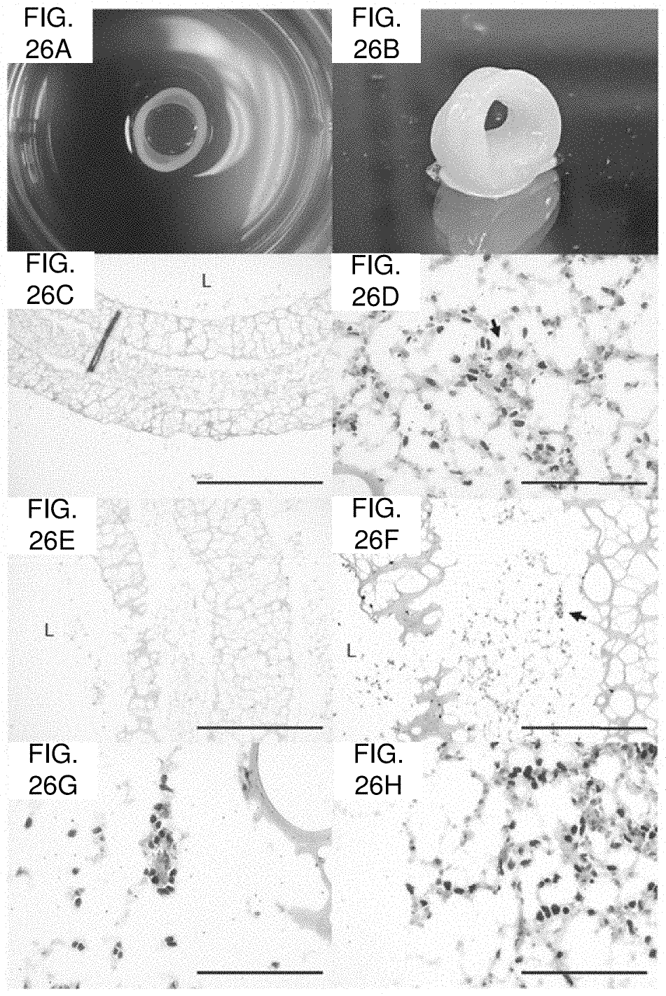

FIG. 26A shows a ND vessel composed of smoot muscles cells, fibrin hydrogel and ox-LDLs. Following the static seeding of M1 macrophages the lumen of the vessel remained open and vessel appeared to still have structural integrity (FIG. 26B). The ND BEBV was frozen down in optimal cutting temperature (OCT) compound and cryo-sectioned. Following sectioning the samples were fixed in 2% formalin solution and stained via Oil Red O solution with a hematoxylin counter stain for the nuclei. The Oil Red O stain was used to visualize the ox-LDLs and macrophages in the tissue rings (FIG. 26C). The presence of the macrophages was determined similarly via the observation of red ox-LDL particles that appear inside of the cells (black arrow) (FIG. 26D). In FIG. 26E we noticed what appeared to be a hole in the lumen fibrin gel with cells that appear to be going inside of the layer containing the SMCs. Looking further in the region we noticed cells huddled around a red particle (FIG. 26F). In the 40× we noticed that this particle ox-LLD due to the red stain (FIG. 26G). These cells are probably the initial macrophages that migrated into the region via engulfing the ox-LDLs in the fibrin hydrogel. This explains the hole in the sample with the trail of cells which probably macrophages as well. Cells closer to the opening of the hole also appear to have red ox-LDL particles inside them confirming that are too macrophages that migrated via the engulfing the opening (FIG. 26H).

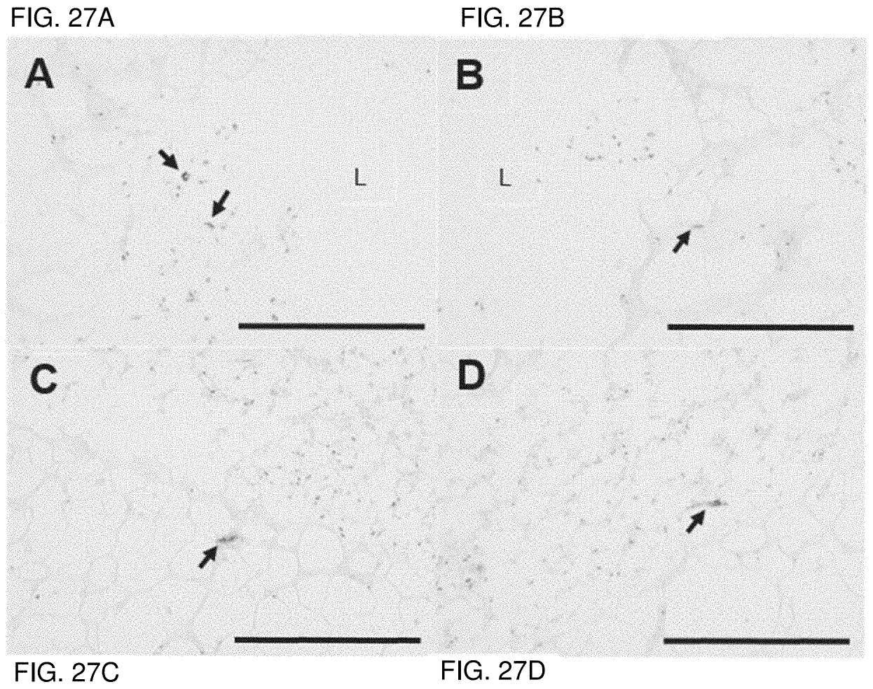

FIGS. 27A-27D shows a von Kossa staining of the ND engineered vessel at 20× magnification. The presence of the CPPs are seen in the lumen of the engineered vessel (black arrows) (FIG. 27A). In FIGS. 27B-27C the presence of the extracellular calcium is seen in the hydrogel and media layer of the vessel. This shows that the presence of the CPPs in the lumen of the engineered vessel induces smooth muscle cell secretion of calcium in the extra cellular matrix of the tissues (black arrows).

### Discussion

In this Example, we have shown the efficacy of augmenting a tissue engineered calcification model of atherosclerosis using a 3-dimensional computer simulation to better understand the effects on the hemodynamics of the system. Importantly, this approach was effective in evaluating the distinctly different occlusion geometries present in atherosclerosis, which are challenging to replicate consistently in existing models. As with all computer models, input of experimental data is ideal for optimizing simulations. This process can be counterintuitive as computer models are touted as a substitution for experimental methods although experimental data is still needed. In actuality, the combination of both methods is the optimum approach.

In the present study, we reported for the first time the successful use of CPPs in a 3-dimensional cylindrical cell culture tissue model to induce atherosclerotic calcification. Other past studies have used CPPs in vitro but only in 2-dimensional cell culture, thus providing limited scientific information. The ability to create a BEBV in our lab and our capabilities to generate computer simulations gave us the unique position of being able to mechanically, hemodynamically and computationally evaluate our tissue engineered atherosclerotic calcification model. With our unique combination of in vitro and computational modelling, the effects of calcification can be better understood.

Tensile data showed an increase in stiffness via calculation of the elastic modulus for CPP rings when compared to rings without CPPs. This trend continues when observing the tensile data for the BEBVs the elastic modulus increases significantly when compared to the control tissues. The atherosclerotic human CIA tensile data showed a larger elastic modulus, when compared to normal human CIA, even though it had a significantly lower ultimate tensile strength. Tissue were only stretched circumferentially and not longitudinal due to the lack of the abundance of human normal and atherosclerotic blood vessels, therefore it made sense to only use uniaxial data for the purpose of this study.

The human CIAs obtained have a low number of samples because of the lack of clinical availability. However, we were fortunate to obtain the CIA samples. The tensile mechanics of the human CIAs proved that stiffening due to atherosclerotic calcification overtime leads to vessels that have a higher elastic modulus when compared to their normal counterparts. Using the measured elastic modulus form the tensile data presented, a ratio can be determined from the atherosclerotic CIA divided by the normal CIA providing a value of 1.55. This value was used to measure the relative stiffness of the engineered model to the actual human CIA. The ratio was 1.96 for the singular rings and 1.33 for the BEBVs. According to this comparison the singular rings did in fact have a higher relative stiffness when compared to the human arteries. One problem that could be brought to attention is that the singular rings are not in a tube-like cylindrical form as the human arteries are. The BEBV ratio is about 85% that of the human artery ratio and is probably not as high as the singular rings due to the cumulative effect the rings have when in vessel form. Instead for failing at a lower strain rate like the CPP singular rings, the CPP BEBVs failed at a higher strain rate. This further proves how forming the rings into vessels increases the overall toughness of the tissues. The results from the tensile mechanics provide some evidence that confirms previous studies conclusions on CPPs inducing SMC extracellular calcium secretion.

Using CPPs to create an in vitro atherosclerotic disease model was validated with a bioreactor-based computer simulation using ANSYS 19. ANSYS provided a powerful CFD (Fluent) and Mechanical (Transient Structural) coupled plate platform if there is experimental uniaxial data. It was determined that the fluid velocity and pressure increased in the CPP BEBV occluded state. These data suggest that CPPs circulating in the blood in CKD patients could be lowering the compliancy of the vessel wall of their arteries [24]. It is interesting to note that when an occlusion is present that the hemodynamic differences become more apparent. On the mechanical perspective, the CPP BEBV normal state displayed a lower deformation then that of the control BEBV. However, the CPP BEBV occluded state only displayed a slightly lower deformation. This could be due to the increased fluid velocity and pressure inside of the CPP occluded state due to the obstruction. The blood appeared to pool before the occlusion, and the pressure built further expanding the vessel. This occurred because of the increased stiffness of the vessel walls causing a loss of compliancy allowing the pressure to increase. Also, the tissues reluctance to expand further resulted in increased fluid velocities at the site of the occlusion. Most notably is the increase in the Reynolds number of the CPP BEBV occluded states compared to that of the control BEBV occluded states, indicating movement towards turbulent flow due to the occlusions. Although the flow remained laminar, the addition of CPPs to stiffen the vessel walls brought the fluid flow increasing the potential for turbulent low. It is possible that at a higher concentration of CPPs, the vessel walls could further stiffen, leading to a complete transition to turbulent state.

The concentric and eccentric occlusions were modelled with geometries that approximated occlusions observed in the body due to the limitations inside of the Fluent toolbox within ANSYS 19 when using a 3-dimensional model. We were only able to correctly map the fluid boundary layers using geometries that allowed for symmetric textures when meshing. A suggestion to create or use a more highly specialized 3D CFD software that will account for the lack symmetry in the fluid and still properly map the boundary layers.

EDM rings were successfully formed. Tensile analysis of the EDM ring compared to the control and CPP rings showed the combined additions of ox-LDL, CPPs and THP-1 cells in the hydrogel change the mechanical properties of the tissue due having its own distinctive stress-strain curve. This is also confirmed via the significant difference between the control and EDM rings. The lack of significance between the CPP rings and the EDM rings shows the stiffing effect of the CPPs in the engineered tissues. This is reinforced via the string presence of the CPPs via the Von Kossa stain. The H&E stain proved that cells a still present in the tissues. Ox-LDLs and some macrophages were visualized via the Oil Red O stain. Not as many macrophages engulfing ox-LDL compared to the ND BEBV due to the embedded THP-1 being undifferentiated.

ND rings and vessels were successfully formed. This engineered vessel represented the 2-cell layer in vitro atherosclerotic disease model pertaining to the smooth muscle cells and endothelial cells. Via an Oil Red O stain macrophage migration and ox-LDL uptake were visualized. A number a macrophage with engulfed ox-LDLs were present after static rotational seeding of differentiated THP-1. Macrophages appeared to have migrated into the media layer via aggressively engulfing some of the fibrin gel containing the ox-LDLs. Via a von Kossa stain CPPs were viewed in the lumen of the engineered vessel. Early smooth muscle cell excretion of extracellular calcium was observed in the hydrogel and tunica media layer of the engineered vessel.

The results from the present study suggest that CPPs can be used to potentially create a late stage atherosclerosis in vitro disease model to test vessel wall compliancy. At the very least, the CPP approach can serve as a viable method to expedite the calcification process inside the walls of a tissue engineered model. In addition, the creation of the intima layer and the statically seeding macrophages proved to be a successful early stage in vitro atherosclerotic disease model seen via the aggressive migration of the macrophages into the media layer.

### SELECT REFERENCES

Dorweiler et al., Thromb Haemost. 95(1):182-189, 2006.
Fearon et al., Toxicology 27:513-522, 2013.
Gu et al., Sci Rep. 9:7461, 2019.
Hussey et al., Nature Rev. Mat. 3:159-173, 2018.
Khodadadie et al., Commun. Biol. 4:378, 2021.
Moore et al., Cell Tissue Bank. 16:249-259, 2015.
Nakazato et al., J. Cardiol. 74:428-435, 2019.
Porzionato et al., Int. J Mol. Sci. 19, 4117, 2018 (79 pages).
Silaghi et al., J Clin Med. 9, 1278, 2020, 16 pages.
Viegas et al., Arterioscler Thromb Vasc Biol. 38:575-587, 2018.
Wada et al., Yonsei Med J. 41(6):740-755, 2000.
Xu et al., Tissue Engin. 14(12):2009-2019, 2008.
Zakiev et al., BioMed Res Internat. 2017, #6198723, 8 pages.
International Patent Application Publication WO 2021/076684, “Improved Mechanical Properties of Implantable Vascular Grafts”.
U.S. Patent Publication No. 2017/0292962 A1, “Method for Determining Propensity for Calcification”

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds and nucleic acid or amino acids sequences referenced herein that are available in a public database, the information in the database entry is incorporated herein by reference as of the date that the database identifier was first included in the text of an application in the priority chain.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8^{th} Edition (Ed. J. Law & R. Rennie, Oxford University Press, 2020).

What is claimed is:

1. An all-natural method to support engineered tissues, essentially as described herein.

2. An all-natural means to provide support and mechanical strength to engineered tissues in order to fabricate all-natural, low immunogenicity tissues in vitro.

3. A bench-top model of atherosclerosis, essentially as described herein.

4. The bench-top model of atherosclerosis of claim 3, which:
is a tissue engineered model of atherosclerosis; and/or
includes early and late state disease processes; and/or
utilizes tissue engineered blood vessel (TEBV).

5. The bench-top model of claim 4, in which stages of atherosclerosis are induced in the TEBV by application of oxidized low-density lipoprotein (oxLDLs) (early stage), followed by macrophage introduction (early stage), and induction of calcification using calcified protein particles (CPPs; late stage).

6. The bench-top model of claim 4, wherein the TEBV is made by a method comprising: selecting a group of cells; selecting an acellular tissue matrix and a hydrogel; and co-culturing the group of cells with the acellular tissue matrix and the hydrogel in a container.

7. The bench-top model of claim 6, wherein one or more of:
the hydrogel comprises a structural protein and at least one component that facilitates the production of extracellular matrix proteins; and/or
wherein the cells are fibroblasts; and/or
the fibroblasts are human dermal fibroblasts; and/or
the cells are in a suspension prior to co-culture; and/or
the cells are trypsinized; and/or
the acellular tissue matrix is a collagen-containing tissue matrix; and/or
the acellular tissue matrix is derived from dermal tissue; and/or
the acellular tissue matrix is completely decellularized; and/or
the acellular tissue matrix is completely or substantially depleted of galactose alpha-1,3-galactose moieties; and/or
the acellular tissue matrix is sterilized prior to use; and/or
the structural protein in the hydrogel comprises or is fibrin; and/or
the hydrogel comprises thrombin and fibrinogen.

8. The bench-top model of claim 7, wherein one or more of:
the at least one component that facilitates the production of extracellular matrix proteins is ascorbic acid, transforming growth factor-beta (TGF-β), human insulin, human fibroblast growth factor (H-FGF), human epidermal growth factor (H-EGF), or a combination thereof; and/or
the hydrogel comprises ascorbic acid and transforming growth factor-beta (TGF-β); and/or
the hydrogel comprises about 0.1% to about 0.5% ascorbic acid and transforming growth factor-beta (TGF-β).

9. The bench-top model of claim 3, further comprising providing a supporting post in contact with the group of cells, the acellular tissue matrix, and the hydrogel.

10. The bench-top model of claim 9, wherein one or more of:
the supporting post is cylinder-shaped and has a dimeter of about 6 mm; and/or
further comprising cutting the acellular tissue matrix into a small piece prior to use; and/or
the small piece is ring-shaped with an inner diameter of about 6 mm, and an outer diameter of 7-8 mm; and/or
the ring-shaped piece of acellular tissue matrix is positioned around the supporting post; and/or
the hydrogel forms a layer on the bottom of the container, and the acellular matrix is positioned on top of the layer; and/or
the acellular tissue matrix is positioned on the bottom of the container, and the hydrogel forms a layer on top of the acellular tissue matrix; and/or
the cells self-assemble into a ring-shaped cellular structure and integrate into the acellular tissue matrix and hydrogel; and/or
further comprising forming additional ring-shaped vascular grafts; and/or
further comprising stacking the ring-shaped vascular grafts into a tubular, vascular-like structure.

11. A kit comprising the bench-top model of claim 3, or another bench-top model described herein.

12. A method of using the kit of claim 11, to investigate a disease process and/or patient treatment option, and/or drug development.

13. A method of making the bench-top model of claim 3, or another bench-top model described herein.

14. A method of inducing calcification in an in vitro atherosclerosis model such as the one in claim 3, comprising introducing an effective amount of calcified protein particles (CPPs) into the model.

15. The method of claim 14, wherein the CPP are introduced into the model during its construction.

16. The bench-top in vitro model of claim 1, in which stages of atherosclerosis are induced by application of oxidized low density lipoprotein (oxLDLs) (early stage), followed by macrophage introduction (early stage), and induction of calcification using calcified protein particles (CPPs; late stage).

17. The method of claim, which uses human cells and/or tissues, and has a substantially three-dimensional structure.
